(19) United States
(12) Patent Application Publication
(10) Pub. No.: US 2020/0331993 A1
(43) Pub. Date: Oct. 22, 2020

(54) DOSAGE AND ADMINISTRATION OF ANTI-C5 ANTIBODIES FOR TREATMENT OF GENERALIZED MYASTHENIA GRAVIS

(71) Applicant: Alexion Pharmaceuticals, Inc., Boston, MA (US)

(72) Inventors: Kenji Fujita, Millburn, NJ (US); Nishi Rampal, Branford, CT (US); Wei-Jian Pan, Pullman, WA (US); Kaushik Patra, Lexington, MA (US)

(21) Appl. No.: 16/791,415

(22) Filed: Feb. 14, 2020

Related U.S. Application Data
(60) Provisional application No. 62/805,350, filed on Feb. 14, 2019.
(60) Provisional application No. 62/814,935, filed on Mar. 7, 2019.

(51) Int. Cl. C07K 16/18; A61P 21/04

(52) U.S. Cl. CPC C07K 16/18; A61P 21/04; A61K 2039/545; C07K 2317/52; C07K 2317/76; C07K 2317/565

(57) ABSTRACT

Provided are methods for clinical treatment of generalized myasthenia gravis (gMG) using an anti-C5 antibody or antigen binding fragment thereof.

### RELATED APPLICATIONS

This application claims the benefit of priority to U.S. Provisional Patent Application No. 62/805,350, filed Feb. 14, 2019, and U.S. Provisional Patent Application No. 62/814,935, filed Mar. 7, 2019, the entire contents of which are incorporated herein by reference for all purposes.

### REFERENCE TO SEQUENCE LISTING SUBMITTED ELECTRONICALLY

The content of the electronically submitted sequence listing in ASCII text file (Name: 701838_AX9-004_ST25.txt; Size: 55 KB; and Date of Creation: Feb. 14, 2020) is incorporated herein by reference in its entirety.

### BACKGROUND

The complement system acts in conjunction with other immunological systems of the body to defend against intrusion of cellular and viral pathogens. There are at least 25 complement proteins, which are found as a complex collection of plasma proteins and membrane cofactors. The plasma proteins make up about 10% of the globulins in vertebrate serum. Complement components achieve their immune defensive functions by interacting in a series of intricate but precise enzymatic cleavage and membrane binding events. The resulting complement cascade leads to the production of products with opsonic, immunoregulatory and lytic functions.

Myasthenia Gravis (MG) is a rare, debilitating, acquired autoimmune neurologic disorder of the neuromuscular junction (NMJ) caused by the failure of neuromuscular transmission, which results from the binding of auto-antibodies (auto-Abs) to proteins involved in signaling at the NMJ. These proteins include the nicotine acetylcholine receptors (AChRs) or, less frequently, a muscle-specific tyrosine kinase (MuSK) involved in AChR clustering.

MG may cause life-threatening respiratory failure, referred to as myasthenic crisis. MG has a prevalence of 14-20 per 100,000 in the U.S., affecting roughly 60,000 Americans. It affects males and females in equal ratio, although the incidence in females peaks in the 3rd decade as compared to males in whom the peak age at onset is in the 6th or 7th decade. About 15% to 20% of subjects will experience a myasthenic crisis during the course of their disease, 75% within 2 years of diagnosis, requiring hospitalization and ventilatory support. Mortality from MG is approximately 4%, mostly due to respiratory failure.

Myasthenia gravis is clinically characterized by weakness and fatigability of voluntary skeletal muscles. MG may initially present with ocular muscle weakness affecting eye and eyelid movement, referred to as ocular MG (oMG). Ten percent of subjects have disease limited to ocular muscles. Ninety percent of subjects have generalized MG, with muscle weakness involving neck, head, spine, bulbar, respiratory or limb muscles. Bulbar weakness refers to muscles controlled by nerves originating from the bulb-like part of the brainstem and manifests as difficulty in talking, chewing, swallowing and control of the head.

Generalized myasthenia gravis (gMG) patients differ from the ocular myasthenia gravis (oMG) population in that neuromuscular inflammation and the resultant clinical findings are not just limited to the ocular muscles, but involve all voluntary muscle groups: the bulbar, respiratory, head, neck, trunk or peripheral muscles with or without involvement of the eyes. Profound weakness and devastating consequences, including slurred speech, dysarthria, dysphagia, disorienting vision, shortness of breath (both with activity and at rest), weakness of the upper and lower extremities, impaired mobility, marked reductions in the ability to perform activities of daily living (ADLs), extreme fatigue and episodes of pulmonary failure requiring mechanical ventilation are hallmarks of gMG. Compared with patients with isolated oMG, patients with gMG have a greater incidence of morbidities and a higher burden of disease. gMG is a rare disorder, having an estimated prevalence between 145 to 278 per million. Patients with gMG suffer from a devastating inflammatory neuromuscular disorder with limited therapeutic options.

Hospitalizations for gMG exacerbations are common, with the need for respiratory support, including mechanical ventilation secondary to respiratory failure (e.g., myasthenic crisis) and gastrointestinal tube placement for nutritional support and prevention of dysphagia-associated aspiration. Patients with more advanced gMG have been reported to experience increased mortality of up to 40% at 10 years following diagnosis.

While there is no cure for MG, there are therapies that reduce muscle weakness and improve neuromuscular function. Current available treatments for myasthenia gravis aim to modulate neuromuscular transmission, inhibit the production or effects of pathogenic antibodies, or inhibit inflammatory cytokines. There is currently no specific treatment that targets the underlying pathophysiology of NMJ injury specifically-anti-AChR antibody-AChR interactions resulting in complement activation via the classical pathway and inflammation, with the resultant destruction of the NMJ. There is no specific treatment that corrects the autoimmune defect in MG. With immunosuppressive therapies (ISTs) representing the current standard of care, which usually combines cholinesterase inhibitors, corticosteroids and immunosuppressive drugs (most commonly azathioprine [AZA], cyclosporine, and mycophenolate mofetil [MMF]), the majority of subjects with MG can have their disease reasonably controlled. These therapies, however, may not be optimal for all patients, and there is a cohort of subjects who do not respond adequately to ISTs, or cannot tolerate ISTs, and those who require repeated treatments with plasma exchange (PE) and/or intravenous immunoglobulin (IVIg) to maintain clinical stability.

In difficult-to-control cases, patients with gMG experience unrelenting inflammation, tissue destruction, and consequent severe morbidities including profound muscle weakness, impaired mobility, shortness of breath, pulmonary failure, extreme fatigue, risk for aspiration, and markedly impaired ADLs. These patients are typically diagnosed in the prime of their adult lives, with a median age of onset ranging from 36 to 60 years. As a result of the morbidities associated with gMG, many patients cannot work or have diminished work capacity, experience difficultly caring for themselves and others, and require assistance speaking, eating, ambulating, breathing and performing ADLs.

Uncontrolled terminal complement activation has been implicated in animal models of experimental autoimmune gMG as well as in other forms of autoimmune neuropathy in humans. Auto-Abs recognize targeted neural or muscle tissues, including the AChR, leading to uncontrolled terminal complement activation at the neural or muscle surface.

Autoantibody-driven uncontrolled terminal complement activation with membrane attack complex (MAC)-dependent lysis and activation, and C5a-dependent inflammation at the NMJ causes AChR loss and failure of neuromuscular transmission. Consistent with this model, both complement component C3 fragments (C3a and C3b) and the MAC C5b-9 have been found in NMJs of MG patients.

As there is no cure for MG, and standard of care is not effective for all patients, there is a need to provide improved methods for treating these patients.

### SUMMARY

Provided herein are compositions and methods for treating generalized myasthenia gravis (gMG) in a human patient, comprising administering to the patient an anti-C5 antibody or antigen binding fragment thereof, wherein the anti-C5 antibody or antigen binding fragment thereof is administered (or is for administration) according to a particular clinical dosage regimen (i.e., at a particular dose amount and according to a specific dosing schedule).

Ravulizumab (also known as antibody BNJ441, ALXN1210 or Ultomiris™) comprises heavy and light chains having the sequences shown in SEQ ID NOs: 14 and 11, respectively, or antigen binding fragments and variants thereof. The terms BNJ441, ALXN1210, ravulizumab and Ultomirism may be used interchangeably throughout this document, but all refer to the same antibody. Accordingly, an exemplary antibody for use in the methods described herein is ravulizumab or an antibody comprising the heavy and light chain complementarity determining regions (CDRs) or variable regions (VRs) of ravulizumab.

In some embodiments, the antibody comprises the CDR1, CDR2, and CDR3 domains of the heavy chain variable (VH) region of ravulizumab having the sequence shown in SEQ ID NO: 12, and the CDR1, CDR2 and CDR3 domains of the light chain variable (VL) region of ravulizumab having the sequence shown in SEQ ID NO: 8. In some embodiments, the antibody comprises CDR1, CDR2 and CDR3 heavy chain sequences as set forth in SEQ ID NOs: 19, 18, and 3, respectively, and CDR1, CDR2 and CDR3 light chain sequences as set forth in SEQ ID NOs: 4, 5, and 6, respectively.

In some embodiments, the antibody comprises VH and VL regions having the amino acid sequences set forth in SEQ ID NO: 12 and SEQ ID NO: 8, respectively. In some embodiments, the antibody comprises a heavy chain constant region asset forth in SEQ ID NO:13. In some embodiments, the antibody comprises a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434, each in EU numbering.

In some embodiments, the antibody comprises CDR1, CDR2 and CDR3 heavy chain sequences as set forth in SEQ ID NOs:19, 18, and 3, respectively, and CDR1, CDR2 and CDR3 light chain sequences as set forth in SEQ ID NOs:4, 5, and 6, respectively and a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434, each in EU numbering.

In some embodiments, the antibody competes for binding with, and/or binds to the same epitope on C5 as, the above-mentioned antibodies. In some embodiments, the antibody has at least about 90% variable region amino acid sequence identity with the above-mentioned antibodies (e.g., at least about 90%, 95% or 99% variable region identity with SEQ ID NO:12 and SEQ ID NO:8).

In some embodiments, the antibody binds to human C5 at pH 7.4 and 25° C. with an affinity dissociation constant (KD) that is in the range 0.1 nM≤KD≤1 nM. In some embodiments, the antibody binds to human C5 at pH 6.0 and 25° C. with a KD≥10 nM. In some embodiments, the [(KD of the antibody or antigen-binding fragment thereof for human C5 at pH 6.0 and at 25° C.)/(KD of the antibody or antigen-binding fragment thereof for human C5 at pH 7.4 and at 25° C.)] of the antibody is greater than 25.

In some embodiments, patients treated according to the methods described herein have been vaccinated against meningococcal infections within 3 years prior to, or at the time of, initiating treatment. In some embodiments, patients who received treatment less than 2 weeks after receiving a meningococcal vaccine are also treated with appropriate prophylactic antibiotics until 2 weeks after vaccination. In some embodiments, patients treated according to the methods described herein are vaccinated against meningococcal serotypes A, C, Y, W135, and/or B.

In some embodiments, the dose of the anti-C5 antibody or antigen binding fragment thereof is based on the weight of the patient. For example, in some embodiments, about 2400 mg, about 2700 mg, about 3000 mg, about 3300 mg, and/or about 3600 mg of the anti-C5 antibody or antigen binding fragment thereof is administered to a patient based on their weight. In some embodiments, 2400 mg or 3000 mg of the anti-C5 antibody or antigen binding fragment thereof is administered to a patient weighing ≥40 to ≤60 kg. In some embodiments, 2700 mg or 3300 mg of the anti-C5 antibody or antigen binding fragment thereof is administered to a patient weighing ≥60 to <100 kg. In some embodiments, 3000 mg or 3600 mg of the anti-C5 antibody or antigen binding fragment thereof is administered to a patient weighing ≥100 kg. In some embodiments, dosage regimens are adjusted to provide the optimum desired response (e.g., an effective response).

In some embodiments, the anti-C5 antibody or antigen binding fragment thereof is administered once on Day 1 of the administration cycle, once on Day 15 of the administration cycle, and every eight weeks thereafter. In some embodiments, the anti-C5 antibody or antigen binding fragment thereof is administered every eight weeks after the administration cycle for an extension period up to two years (e.g., at a dose of 3000 mg, 3300 mg, or 3600 mg).

In some embodiments, the anti-C5 antibody or antigen binding fragment thereof is administered for one or more administration cycles. In some embodiments, the administration cycle is 26 weeks. In some embodiments, the treatment comprises at least 1, 2, 3, 4, 5, 6, 7, 8, 9, 10, or 11 cycles. In some embodiments, the treatment continues for the lifetime of the human patient.

In some embodiments, a patient switches from receiving one C5 inhibitor to a different C5 inhibitor during the course of treatment. Different anti-C5 antibodies may be administered during separate treatment periods. For example, in some embodiments, a method of treating a human patient having a complement-associated disorder (e.g., generalized myasthenia gravis (gMG)) who is being treated with eculizumab is provided, the method comprising discontinuing treatment with eculizumab and switching the patient to treatment with an alternative complement inhibitor. For example, in some embodiments, the patient is treated with eculizumab during a treatment period (e.g., for 26 weeks), followed by treatment with another anti-C5 antibody (e.g., ravulizumab) during an extension period. In some embodiments, eculizumab is administered to the patient at a dose of 900 mg on Days 1, 8, 15, and 22 of the administration cycle during an induction phase, followed by a maintenance dose of 1200 mg of eculizumab on Day 19 of the administration cycle and every two weeks thereafter (e.g., for a total of 26 weeks), followed by treatment with ravulizumab for an extension period of up to two years. In some embodiments, a method of treating a human patient having a complement-associated disorder who is being treated with ravulizumab is provided, the method comprising discontinuing treatment with ravulizumab and switching the patient to treatment with an alternative complement inhibitor. For example, the patient is treated with ravulizumab during a treatment period (e.g., for 26 weeks), followed by treatment with another anti-C5 antibody (e.g., eculizumab) during an extension period.

Exemplary alternative complement inhibitors include, but are not limited to antibodies, or antigen-binding fragments thereof, small molecules, polypeptides, polypeptide analogs, peptidomimetics, siRNA and aptamers. In some embodiments, the alternative complement inhibitor inhibits one or more of complement components C1, C2, C3, C4, C5, C6, C7, C8, C9, Factor D, Factor B, properdin, MBL, MASP-1, MASP-2, or biologically active fragments thereof. In some embodiments, the alternative complement inhibitor inhibits one or both of the generation of the anaphylatoxic activity associated with C5a and/or the assembly of the membrane attack complex associated with C5b. In some embodiments, the alternative complement inhibitor is selected from the group consisting of CR1, LEX-CR1, MCP, DAF, CD59, Factor H, cobra venom factor, FUT-175, complestatin, and K76 COOH.

In some embodiments, the treatment regimens described are sufficient to maintain particular serum trough concentrations of the anti-C5 antibody or antigen binding fragment thereof. For example, in some embodiments, the treatment maintains a serum trough concentration of the anti-C5 antibody or antigen binding fragment thereof of 50, 55, 60, 65, 70, 75, 80, 85, 90, 95, 100, 105, 110, 115, 120, 125, 130, 135, 140, 145, 150, 155, 160, 165, 170, 175, 180, 185, 190, 200, 205, 210, 215, 220, 225, 230, 240, 245, 250, 255, 260, 265, 270, 280, 290, 300, 305, 310, 315, 320, 325, 330, 335, 340, 345, 350, 355, 360, 365, 370, 375, 380, 385, 390, 395, or 400 μg/ml or greater. In some embodiments, the treatment maintains a serum trough concentration of the anti-C5 antibody or antigen binding fragment thereof of 100 μg/ml or greater. In some embodiments, the treatment maintains a serum trough concentration of the anti-C5 antibody or antigen binding fragment thereof of 150 μg/mI or greater. In some embodiments, the treatment maintains a serum trough concentration of the anti-C5 antibody or antigen binding fragment thereof of 200 μg/ml or greater. In some embodiments, the treatment maintains a serum trough concentration of the anti-C5 antibody or antigen binding fragment thereof of 250 μg/ml or greater. In some embodiments, the treatment maintains a serum trough concentration of the anti-C5 antibody or antigen binding fragment thereof of 300 μg/ml or greater. In some embodiments, the treatment maintains a serum trough concentration of the anti-C5 antibody or antigen binding fragment thereof of between 100 μg/ml and 200 μg/ml. In some embodiments, the treatment maintains a serum trough concentration of the anti-C5 antibody or antigen binding fragment thereof of about 175 μg/ml.

In some embodiments, to obtain an effective response, the anti-C5 antibody is administered to the patient in an amount and with a frequency to maintain at least 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, or 260 μg of antibody per milliliter of the patient's blood. In some embodiments, the anti-C5 antibody is administered to the patient in an amount and with a frequency to maintain between 50 μg and 250 μg of antibody per milliliter of the patient's blood. In some embodiments, the anti-C5 antibody is administered to the patient in an amount and with a frequency to maintain between 100 μg and 200 μg of antibody per milliliter of the patient's blood. In some embodiments, the anti-C5 antibody is administered to the patient in an amount and with a frequency to maintain about 175 μg of antibody per milliliter of the patient's blood.

In some embodiments, to obtain an effective response, the anti-C5 antibody is administered to the patient in an amount and with a frequency to maintain a minimum free C5 concentration. For example, in some embodiments, the anti-C5 antibody is administered to the patient in an amount and with a frequency to maintain a free C5 concentration of 0.2 μg/mL, 0.3 μg/mL, 0.4 μg/mL, 0.5 μg/mL or below. In some embodiments, the anti-C5 antibody is administered to the patient in an amount and with a frequency to maintain a free C5 concentration of 0.309 to 0.5 μg/mL or below. In some embodiments, the treatment described herein reduces free C5 concentration by greater than 99% throughout the treatment period. In some embodiments, the treatment reduces free C5 concentration greater than 99.5% throughout the treatment period.

The anti-C5 antibodies or antigen binding fragments thereof can be administered to a patient by any suitable means. In some embodiments, the antibodies are formulated for intravenous administration.

The efficacy of the treatment methods provided herein can be assessed using any suitable means. In some embodiments, for a gMG patient, the treatment produces at least one therapeutic effect selected from the group consisting of but not limited to a reduction or cessation in inflammation, tissue destruction, profound weakness, slurred speech, dysarthria, dysphagia, disorienting vision, shortness of breath (both with activity and at rest), weakness of the upper and lower extremities, impaired mobility, marked reductions in the ability to perform activities of daily living (ADLs), extreme fatigue, and episodes of pulmonary failure requiring mechanical ventilation. In another embodiment, the patient has a clinically meaningful improvement (reduction) in one or more measurements of gMG severity selected from the group consisting of MG-ADL, QMG, MG-QOL15r, Neuro-QOL Fatigue, EQ-5D-5L, MGFA-PIS and/or MGC.

In some embodiments, the treatment results in terminal complement inhibition.

In some embodiments, this disclosure provides a method comprising administering a therapeutically effective amount of ravulizumab to a patient, wherein the patient is positive for auto-antibodies binding to nicotinic acetylcholine receptor (anti-AChR) and shows marked generalized weakness or bulbar signs and symptoms of myasthenia gravis, and wherein the patient is administered ravulizumab for at least 26 weeks. In some embodiments, the patient had previously received therapy for myasthenia gravis including anticholinesterase inhibitor therapy and immunosuppressant therapy (IST) and requires chronic plasma exchange or chronic IVIg to maintain clinical stability.

In some embodiments, the patient being treated by the methods provided herein experiences a clinically meaningful improvement (reduction) in Myasthenia Gravis Activities of Daily Living (MG-ADL) score after 26 weeks of treatment. In some embodiments, the treatment effect will be estimated by the difference in means between the ravulizumab group and placebo group in the change from Baseline in MG-ADL total score at Week 26 irrespective of rescue therapy. A lower value of the corresponding estimate will indicate a beneficial treatment effect. In some embodiments, rescue therapy will be allowed when a patient's health is in jeopardy, if rescue therapy was not administered (e.g., emergent situations), or if a patient experiences clinical deterioration, as defined herein. In some embodiments, rescue therapy includes high-dose corticosteroids, PP/PE or IVIg. In some embodiments, the clinically meaningful improvement the patient experiences is at least a 3 point reduction in the patient's MG-ADL score after 26 weeks of treatment. In some embodiments, the treatment effect corresponding to the dichotomous endpoint of the MG-ADL 3-point response at Week 26 irrespective of rescue therapy will be estimated by the odds ratio (OR) of the proportions of the corresponding endpoint in the ravulizumab group compared with the placebo group.

In some embodiments, the patient being treated by the methods provided herein experiences a clinically meaningful improvement (reduction) in quantitative Myasthenia Gravis score (QMG) after 26 weeks of treatment. In some embodiments, the treatment effect corresponding to the change from Baseline continuous endpoints will be estimated by the difference in means between the ravulizumab group and placebo group in the change from Baseline in QMG score at Week 26 irrespective of rescue therapy. A lower value of the corresponding estimate will indicate a beneficial treatment effect. In some embodiments, the clinically meaningful improvement the patient experiences is at least a 5 point reduction in the patient's QMG score after 26 weeks of treatment. In some embodiments, the treatment effect corresponding to the dichotomous endpoint of the QMG 5-point response at Week 26 irrespective of rescue therapy will be estimated by the odds ratio (OR) of the proportions of the corresponding endpoint in the ravulizumab group compared with the placebo group.

In some embodiments, the patient being treated by the methods provided herein experiences a clinically meaningful improvement (reduction) in Myasthenia Gravis Composite (MGC) score after 26 weeks of treatment. In some embodiments, the treatment effect corresponding to the change from Baseline continuous endpoints will be estimated by the difference in means between the ravulizumab group and placebo group in the change from Baseline in MGC score at Week 26 irrespective of rescue therapy. A lower value of the corresponding estimate will indicate a beneficial treatment effect.

In some embodiments, the patient being treated by the methods provided herein experiences a clinically meaningful improvement (reduction) in quality of life as measured by the Revised 15-Component Myasthenia Gravis Quality of Life (MG-QOL15r) score after 26 weeks of treatment. In some embodiments, the treatment effect corresponding to the change from Baseline continuous endpoints will be estimated by the difference in means between the ravulizumab group and placebo group in the change from Baseline in MG-QOL15r score at Week 26 irrespective of rescue therapy. A lower value of the corresponding estimate will indicate a beneficial treatment effect.

In some embodiments, the patient being treated by the methods provided herein experiences a clinically meaningful improvement (reduction) in neuro-fatigue as measured by the Neuro-QOL Fatigue score after 26 weeks of treatment. In some embodiments, the treatment effect corresponding to the change from Baseline continuous endpoints will be estimated by the difference in means between the ravulizumab group and placebo group in the change from Baseline in Neuro-QOL score at Week 26 irrespective of rescue therapy. A lower value of the corresponding estimate will indicate a beneficial treatment effect.

In some embodiments, the patient being treated by the methods provided herein experiences a clinically meaningful improvement (increase) in health status as measured by the EQ-5D-5L health status score after 26 weeks of treatment. In some embodiments, the patient being treated by the methods provided herein experiences a clinically meaningful improvement (increase) in health status as measured by the EQ-5D-5L index score after 26 weeks of treatment. In some embodiments, the patient being treated by the methods provided herein experiences a clinically meaningful improvement (increase) in health status as measured by the EQ-5D-5L VAS score after 26 weeks of treatment. In some embodiments, the treatment effect corresponding to the change from Baseline continuous endpoints will be estimated by the difference in means between the ravulizumab group and placebo group in the change from Baseline in EQ-5D-5L health status score (e.g., EQ-5D-5L index score or EQ-5D-5L VAS score at Week 26), irrespective of rescue therapy. A lower value of the corresponding estimate will indicate a beneficial treatment effect.

In some embodiments, the patient being treated by the methods provided herein experiences a clinically meaningful improvement (increase) in health status as measured by the MGFA-PIS score after 26 weeks of treatment. The treatment effect corresponding to the MGFA-PIS endpoint will be estimated by the proportional odds ratio (OR) of the cumulative proportions over the ordinal categories (starting from the best outcome) of this endpoint in the ravulizumab group compared with the placebo group at Week 26, irrespective of rescue therapy. An estimate of OR>1 will indicate a beneficial treatment effect.

In some embodiments, the patient being treated by the methods provided herein experiences a clinically meaningful improvement (increase) in health status as measured by the reduced incidence of all-cause hospitalization or clinical deterioration, as defined herein, after 26 weeks of treatment. In some embodiments, the treatment effect corresponding to the dichotomous endpoint of the all-cause hospitalization or clinical deterioration, as defined herein, over 26 weeks irrespective of rescue therapy will be estimated by the odds ratio (OR) of the proportions of the corresponding endpoint in the ravulizumab group compared with the placebo group. An estimate of OR<1 corresponding to the composite hospitalization endpoint will indicate a beneficial treatment effect, likewise an estimate of OR>1 corresponding responder endpoints will indicate a beneficial treatment effect.

In some embodiments, this disclosure provides a method of treating generalized myasthenia gravis in a patient in need thereof comprising administering ravulizumab to the patient, wherein the patient is positive for auto-antibodies binding to nicotinic acetylcholine receptor (anti-AChR) and shows marked generalized weakness or bulbar signs and symptoms of myasthenia gravis while receiving therapy for myasthenia gravis including anticholinesterase inhibitor therapy and immunosuppressant therapy (IST) or requires chronic plasma exchange or chronic IVIg to maintain clinical stability; wherein ravulizumab is administered using a phased dosing schedule as defined herein, and wherein the patient has a clinically meaningful improvement (reduction) in at least one measurement of generalized myasthenia gravis severity selected from the group consisting of MG-ADL, QMG, MG-QOL15r, Neuro-QOL Fatigue, EQ-5D-5L, MGFA-PIS and/or MGC.

In some embodiments, this disclosure provides a method of treating generalized myasthenia gravis in a patient in need thereof comprising administering ravulizumab to the patient, wherein the patient is positive for auto-antibodies binding to nicotinic acetylcholine receptor (anti-AChR) and shows marked generalized weakness or bulbar signs and symptoms of myasthenia gravis while receiving therapy for myasthenia gravis including anticholinesterase inhibitor therapy and immunosuppressant therapy (IST) and requires chronic plasma exchange or chronic IVIg to maintain clinical stability; wherein ravulizumab is administered using a phased dosing schedule as disclosed herein, and wherein the patient has a clinically meaningful improvement (reduction) in two measurements of generalized myasthenia gravis severity selected from the group consisting of MG-ADL, QMG, MG-QOL15r, Neuro-QOL Fatigue, EQ-5D-5L, MGFA-PIS and/or MGC.

In some embodiments, this disclosure provides a method of treating generalized myasthenia gravis in a patient in need thereof comprising administering ravulizumab to the patient, wherein the patient is positive for auto-antibodies binding to nicotinic acetylcholine receptor (anti-AChR) and shows marked generalized weakness or bulbar signs and symptoms of myasthenia gravis while receiving therapy for myasthenia gravis including anticholinesterase inhibitor therapy and immunosuppressant therapy (IST) or requires chronic plasma exchange or chronic IVIg to maintain clinical stability; wherein ravulizumab is administered using a phased dosing schedule as disclosed herein, and wherein the patient has a clinically meaningful improvement (reduction) in three measurements of generalized myasthenia gravis severity selected from the group consisting of MG-ADL, QMG, MG-QOL15r, Neuro-QOL Fatigue, EQ-5D-5L, MGFA-PIS and/or MGC. In some embodiments, the patient has a clinically meaningful improvement (reduction) in four measurements of generalized myasthenia gravis severity selected from the group consisting of MG-ADL, QMG, MG-QOL15r, Neuro-QOL Fatigue, EQ-5D-5L, MGFA-PIS and/or MGC. In some embodiments, the patient has a clinically meaningful improvement (reduction) in five measurements of generalized myasthenia gravis severity, wherein the five measurements of generalized myasthenia gravis severity are MG-ADL, QMG, MG-QOL15r, Neuro-QOL Fatigue, EQ-5D-5L, MGFA-PIS and/or MGC. In some embodiments, the patient has a clinically meaningful improvement (reduction) in six measurements of generalized myasthenia gravis severity, wherein the five measurements of generalized myasthenia gravis severity are MG-ADL, QMG, MG-QOL 15r, Neuro-QOL Fatigue, EQ-5D-5L, MGFA-PIS and/or MGC. In some embodiments, the patient has a clinically meaningful improvement (reduction) in seven measurements of generalized myasthenia gravis severity, wherein the five measurements of generalized myasthenia gravis severity are MG-ADL, QMG, MG-QOL15r, Neuro-QOL Fatigue, EQ-5D-5L, MGFA-PIS and/or MGC.

In some embodiments, this disclosure provides a method of treating generalized myasthenia gravis in a patient in need thereof comprising administering ravulizumab by intravenous infusion. In some embodiments, ravulizumab is administered subcutaneously. In some embodiments, the ravulizumab comprises a heavy chain amino acid sequence according to SEQ ID NO: 12 and a light chain amino acid sequence according to SEQ ID NO: 11. In some embodiments, the ravulizumab is ravulizumab variant comprising a heavy chain amino acid sequence according to SEQ ID NO: 14 and a light chain amino acid sequence according to SEQ ID NO: 11.

In some embodiments, this disclosure provides a method of treating generalized myasthenia gravis in a patient in need thereof comprising administering an anti-C5 antibody or antigen binding fragment thereof, wherein the antibody is an anti-C5 antibody or an antigen binding fragment thereof comprising a heavy chain variable region amino acid sequence according to SEQ ID NO: 27 and a light chain variable region amino acid sequence according to SEQ ID NO: 28. In some embodiments, the antibody is an anti-C5 antibody or an antigen binding fragment thereof comprising a heavy chain variable region amino acid sequence according to SEQ ID NO: 35 and a light chain variable region amino acid sequence according to SEQ ID NO: 36. In some embodiments, the antibody is an anti-C5 antibody or antigen binding fragment thereof comprising a heavy chain variable region amino acid sequence according to SEQ ID NO: 43 and a light chain variable region amino acid sequence according to SEQ ID NO: 44. In some embodiments, the antibody is an anti-C5 antibody or antigen binding fragment thereof comprising a heavy chain variable region amino acid sequence according to SEQ ID NO: 45 and a light chain variable region amino acid sequence according to SEQ ID NO: 46.

In some embodiments, this disclosure provides a method of treating generalized myasthenia gravis in a patient in need thereof comprising administering a therapeutically effective amount of ravulizumab is maintained at a concentration of between 50-100 μg/mL in the patient's serum.

In some embodiments, this disclosure provides a method of treating generalized myasthenia gravis in a patient in need thereof comprising administering a therapeutically effective amount of ravulizumab, wherein the patient experiences a discontinuation in the administration of one or more IST following at least 26 weeks of treatment.

In some embodiments, this disclosure provides a method of treating generalized myasthenia gravis in a patient in need thereof comprising administering a therapeutically effective amount of ravulizumab, wherein the patient experiences a reduction in the need for chronic plasma exchange or chronic IVIg to maintain clinical stability following at least 26 weeks of treatment.

In some embodiments, this disclosure provides a method of treating generalized myasthenia gravis in a patient in need thereof comprising administering a therapeutically effective amount of ravulizumab, wherein the patient no longer requires chronic plasma exchange or chronic IVIg to maintain clinical stability following at least 26 weeks of treatment.

In some embodiments, this disclosure provides a method of treating generalized myasthenia gravis in a patient in need thereof comprising administering a therapeutically effective amount of ravulizumab, wherein the patient experiences a reduction in the need for chronic plasma exchange or chronic IVIg to maintain clinical stability following at least 26 weeks of treatment.

In some embodiments, this disclosure provides a composition for use in a method of treating myasthenia gravis (MG) in a human patient, the treatment comprising administering to the patient an effective amount of the composition, wherein the composition comprises an antibody or an antigen binding fragment thereof comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively.

In some embodiments, the antibody or the antigen binding fragment thereof comprises a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434, each in EU numbering.

In some embodiments, the composition comprising the antibody or the antigen binding fragment thereof is administered: (a) once on Day 1 of the administration cycle at a dose of: 2400 mg to a patient weighing ≥40 to <60 kg, 2700 mg to a patient weighing ≥60 to <100 kg, or 3000 mg to a patient weighing ≥100 kg; and (b) on Day 15 of the administration cycle and every eight weeks thereafter at a dose of 3000 mg to a patient weighing ≥40 to <60 kg, 3300 mg to a patient weighing ≥60 to <100 kg, or 3600 mg to a patient weighing ≥100 kg.

In some embodiments, the antibody or the antigen binding fragment thereof comprises the heavy chain variable region of SEQ ID NO:12 and the light chain variable region of SEQ ID NO:8. In some embodiments, the antibody or the antigen binding fragment thereof further comprises the heavy chain constant region of SEQ ID NO:13.

In some embodiments, the antibody or the antigen binding fragment thereof comprises a heavy chain polypeptide comprising the amino acid sequence of SEQ ID NO:14 and the light chain polypeptide comprising the amino acid sequence of SEQ ID NO:11.

In some embodiments, the antibody or the antigen binding fragment thereof binds to human C5 at pH 7.4 and 25° C. with an affinity dissociation constant (KD) that is in the range 0.1 nM≤KD≤1 nM. In some embodiments, the antibody or the antigen binding fragment thereof, binds to human C5 at pH 6.0 and 25° C. with a KD≥10 nM.

In some embodiments, the antibody or the antigen binding fragment thereof is administered to a patient weighing ≥40 to <60 kg: (a) once on Day 1 of the administration cycle at a loading dose of 2400 mg; and (b) on Day 15 of the administration cycle and every eight weeks thereafter at a maintenance dose of 3000 mg.

In some embodiments, the antibody or the antigen binding fragment thereof is administered to a patient weighing ≥60 to <100 kg: (a) once on Day 1 of the administration cycle at a loading dose of 2700 mg; and (b) on Day 15 of the administration cycle and every eight weeks thereafter at a maintenance dose of 3300 mg.

In some embodiments, the antibody or the antigen binding fragment thereof is administered to a patient weighing ≥100 kg: (a) once on Day 1 of the administration cycle at a loading dose of 3000 mg; and (b) on Day 15 of the administration cycle and every eight weeks thereafter at a maintenance dose of 3600 mg.

In some embodiments, treatment with the antibody or the antigen binding fragment thereof maintains a serum trough concentration of the antibody or the antigen binding fragment thereof of 100 μg/mL or greater during the administration cycle. In some embodiments, treatment with the antibody or the antigen binding fragment thereof maintains a serum trough concentration of the antibody or the antigen binding fragment thereof of 200 μg/mL or greater during the administration cycle.

In some embodiments, treatment with the antibody or the antigen binding fragment thereof maintains a free antibody or antigen binding fragment thereof concentration of 0.309 to 0.5 μg/mL or less.

In some embodiments the antibody or the antigen binding fragment thereof is administered at a dose of 3000 mg, 3300 mg or 3600 mg every eight weeks after the administration cycle for up to two years.

In some embodiments, the antibody or the antigen binding fragment thereof is formulated for intravenous administration.

In some embodiments, the patient treated with the antibody or the antigen binding fragment thereof has not previously been treated with a complement inhibitor.

In some embodiments, the administration cycle is a total of 26 weeks of treatment.

In some embodiments, treatment with the antibody or the antigen binding fragment thereof results in terminal complement inhibition.

In some embodiments, treatment with the antibody or the antigen binding fragment thereof results in the patient experiencing a clinically meaningful improvement (reduction) in Myasthenia Gravis Activities of Daily Living (MG-ADL) score after 26 weeks of treatment. In some embodiments, the clinically meaningful improvement the patient experiences is at least a 3 point reduction in the patient's MG-ADL score after 26 weeks of treatment.

In some embodiments, treatment with the antibody or the antigen binding fragment thereof results a clinically meaningful improvement (reduction) in quantitative Myasthenia Gravis score (QMG) after 26 weeks of treatment. In some embodiments, the clinically meaningful improvement the patient experiences is at least a 5 point reduction in the patient's QMG after 26 weeks of treatment.

In some embodiments, treatment with the antibody or the antigen binding fragment thereof results in a clinically meaningful improvement (reduction) in Myasthenia Gravis Composite (MGC) score after 26 weeks of treatment.

In some embodiments, treatment with the antibody or the antigen binding fragment thereof results in a clinically meaningful improvement (reduction) in quality of life as measured by Myasthenia Gravis Quality of Life (MG-QOL15r) score after 26 weeks of treatment.

In some embodiments, treatment with the antibody or the antigen binding fragment thereof results in a clinically meaningful improvement (reduction) in neuro-fatigue as measured by Neuro-QOL Fatigue score after 26 weeks of treatment.

In some embodiments, treatment with the antibody or the antigen binding fragment thereof results in a clinically meaningful improvement (reduction) in health status as measured by the Euro Quality of Life (EQ-5D-5L) health status score after 26 weeks of treatment.

In some embodiments, treatment with the antibody or the antigen binding fragment thereof results in a clinically meaningful improvement (reduction) in the Myasthenia Gravis Foundation of America (MGFA) Post-Intervention Status (PIS) after 26 weeks of treatment.

In some embodiments, the myasthenia gravis is generalized myasthenia gravis (gMG). In some embodiments, the gMG patient is anti AChR antibody positive.

In some embodiments, the antibody is ravulizumab.

In some embodiments, a kit for treating myasthenia gravis (MG) in a human patient is provided, the kit comprising: (a) a dose of the antibody or the antigen binding fragment thereof comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8; and (b) instructions for using the antibody or the antigen binding fragment thereof in the method of any one of the preceding claims.

In some embodiments, the antibody or the antigen binding fragment thereof of kit comprises a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434, each in EU numbering.

In some embodiments, the antibody or the antigen binding fragment thereof of the kit is administered to a patient weighing ≥40 to <60 kg: (a) once on Day 1 of the administration cycle at a loading dose of 2400 mg; and (b) on Day 15 of the administration cycles and every eight weeks thereafter at a maintenance does of 3000 mg.

In some embodiments, the antibody or the antigen binding fragment thereof of the kit is administered to a patient weighing ≥60 to <100 kg: (a) once on Day 1 of the administration cycle at a dose of 2700 mg; and (b) on Day 15 of the administration cycles and every eight weeks thereafter at a maintenance does of 3300 mg.

In some embodiments, the antibody or the antigen binding fragment thereof of the kit is administered to a patient weighing ≥100 kg: (a) once on Day 1 of the administration cycle at a dose of 3000 mg; and (b) on Day 15 of the administration cycles and every eight weeks thereafter at a maintenance does of 3600 mg.

In some embodiments, the antibody is ravulizumab.

In some embodiments, the disclosure provides an antibody comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8 is provided, for administration in a treatment cycle.

In some embodiments, the antibody comprises a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434, each in EU numbering.

In some embodiments, the antibody is administered: (a) once on Day 1 of the administration cycle at a dose of: 2400 mg to a patient weighing ≥40 to <60 kg, 2700 mg to a patient weighing ≥60 to <100 kg, or 3000 mg to a patient weighing ≥100 kg; and (b) on Day 15 of the administration cycle and every eight weeks thereafter at a dose of 3000 mg to a patient weighing ≥40 to <60 kg, 3300 mg to a patient weighing ≥60 to <100 kg, or 3600 mg to a patient weighing ≥100 kg.

In some embodiments, the antibody is determined to be safe, tolerable, efficacious and sufficiently non-immunogenic after multiple IV doses for use in MG patients.

In some embodiments, the antibody is ravulizumab.

In some embodiments, a method of treating a human patient with MG is provided, the method comprising administering to the patient an effective amount of an antibody or an antigen binding fragment thereof comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively.

In some embodiments, the antibody or the antigen binding fragment thereof comprises a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434, each in EU numbering.

In some embodiments, the antibody or the antigen binding fragment thereof is administered: (a) once on Day 1 of the administration cycle at a dose of: 2400 mg to a patient weighing ≥40 to <60 kg, 2700 mg to a patient weighing ≥60 to <100 kg, or 3000 mg to a patient weighing ≥100 kg; and (b) on Day 15 of the administration cycle and every eight weeks thereafter at a dose of 3000 mg to a patient weighing ≥40 to <60 kg, 3300 mg to a patient weighing ≥60 to <100 kg, or 3600 mg to a patient weighing ≥100 kg.

In some embodiments, the antibody or the antigen binding fragment thereof comprises the heavy chain variable region of SEQ ID NO:12 and the light chain variable region of SEQ ID NO:8. In some embodiments, the antibody or the antigen binding fragment thereof further comprises the heavy chain constant region of SEQ ID NO:13.

In some embodiments, the antibody or the antigen binding fragment thereof comprises a heavy chain polypeptide comprising the amino acid sequence of SEQ ID NO:14 and the light chain polypeptide comprising the amino acid sequence of SEQ ID NO:11.

In some embodiments, the antibody or the antigen binding fragment thereof binds to human C5 at pH 7.4 and 25° C. with an affinity dissociation constant (K_{D}) that is in the range 0.1 nM≤K_{D ≤}1 nM. In some embodiments, the antibody or the antigen binding fragment thereof, binds to human C5 at pH 6.0 and 25° C. with a K_{D} ≥10 nM.

In some embodiments, the antibody or the antigen binding fragment thereof is administered to a patient weighing ≥40 to <60 kg: (a) once on Day 1 of the administration cycle at a dose of 2400 mg; and (b) on Day 15 of the administration cycle and every eight weeks thereafter at a dose of 3000 mg.

In some embodiments, the antibody or the antigen binding fragment thereof is administered to a patient weighing ≥60 to <100 kg: (a) once on Day 1 of the administration cycle at a dose of 2700 mg; and (b) on Day 15 of the administration cycle and every eight weeks thereafter at a dose of 3300 mg.

In some embodiments, the antibody or the antigen binding fragment thereof is administered to a patient weighing ≥100 kg: (a) once on Day 1 of the administration cycle at a dose of 3000 mg; and (b) on Day 15 of the administration cycle and every eight weeks thereafter at a dose of 3600 mg.

In some embodiments, treatment with the antibody or the antigen binding fragment thereof maintains a serum trough concentration of the antibody or the antigen binding fragment thereof of 100 μg/mL or greater during the administration cycle. In some embodiments, treatment with the antibody or the antigen binding fragment thereof maintains a serum trough concentration of the antibody or the antigen binding fragment thereof of 200 μg/mL or greater during the administration cycle.

In some embodiments, treatment with the antibody or the antigen binding fragment thereof maintains a free antibody or antigen binding fragment concentration of 0.309 to 0.5 μg/mL or less.

In some embodiments, the antibody or the antigen binding fragment thereof is administered at a dose of 3000 mg, 3300 mg, or 3600 mg every eight weeks after the administration cycle for up to two years.

In some embodiments, the antibody or the antigen binding fragment thereof is formulated for intravenous administration.

In some embodiments, the patient has not previously been treated with a complement inhibitor.

In some embodiments, the administration cycle is a total of 26 weeks of treatment. In some embodiments, the treatment results in terminal complement inhibition.

In some embodiments, treatment with the antibody or the antigen binding fragment thereof results in the patient experiencing a clinically meaningful improvement (reduction) in Myasthenia Gravis Activities of Daily Living (MG-ADL) score after 26 weeks of treatment. In some embodiments, the clinically meaningful improvement the patient experiences is at least a 3 point reduction in the patient's MG-ADL score after 26 weeks of treatment.

In some embodiments, treatment with the antibody or the antigen binding fragment thereof results a clinically meaningful improvement (reduction) in quantitative Myasthenia Gravis score (QMG) after 26 weeks of treatment. In some embodiments, the clinically meaningful improvement the patient experiences is at least a 5 point reduction in the patient's QMG after 26 weeks of treatment.

In some embodiments, treatment with the antibody or the antigen binding fragment thereof results a clinically meaningful improvement (reduction) in Myasthenia Gravis Composite (MGC) score after 26 weeks of treatment.

In some embodiments, treatment with the antibody or the antigen binding fragment thereof results a clinically meaningful improvement (reduction) in quality of life as measured by Myasthenia Gravis Quality of Life (MG-QOL15r) score after 26 weeks of treatment.

In some embodiments, treatment with the antibody or the antigen binding fragment thereof results a clinically meaningful improvement (reduction) in neuro-fatigue as measured by Neuro-QOL Fatigue score after 26 weeks of treatment.

In some embodiments, treatment with the antibody or the antigen binding fragment thereof results a clinically meaningful improvement (reduction) in health status as measured by the Euro Quality of Life (EQ-5D-5L) health status score after 26 weeks of treatment.

In some embodiments, treatment with the antibody or the antigen binding fragment thereof results a clinically meaningful improvement (reduction) in the Myasthenia Gravis Foundation of America (MGFA) Post-Intervention Status (PIS) after 26 weeks of treatment.

In some embodiments, the myasthenia gravis is generalized myasthenia gravis (gMG). In some embodiments, the gMG patient is anti-AChR antibody positive.

In some embodiments, the antibody is ravulizumab.

Further, the disclosure encompasses any of the above embodiments being used with any other of the above embodiments in any combination.

### BRIEF DESCRIPTION OF THE DRAWINGS

FIG. 1 is a schematic depicting the design of a Phase III ALXN1210-MG-306 clinical trial in gMG patients.

FIG. 2 is a schematic depicting the every 8 week dosage regimen for ravulizumab versus the every 2 week dosage regimen for eculizumab including the actual infusion days, for patients participating in the Phase III ALXN1210-MG-306 study.

FIG. 3A, FIG. 3B, and FIG. 3C are the European Quality of Life Survey (EQ-5D-5L) health status questionnaire used in the clinical trial disclosed herein.

FIG. 4 is the Columbia-Suicide Severity Rating Scale (C-SSRS) as measured at the patient's baseline/screening.

FIG. 5 is the Columbia-Suicide Severity Rating Scale (C-SSRS) as measured since the time of the patient's last visit.

### DETAILED DESCRIPTION

As used herein, the term “subject” or “patient” is a human patient (e.g., a patient having generalized myasthenia gravis (gMG)). As used herein, the terms “subject” and “patient” are interchangeable.

As used herein, the phrase “requires chronic plasma exchange” refers to the use of plasma exchange therapy on a patient on a regular basis for the management of muscle weakness at least every 3 months over the last 12 months.

As used herein, the phrase “requires chronic IVIg” refers to the use of IVIg therapy on a patient on a regular basis for the management of muscle weakness at least every 3 months over the last 12 months.

As used herein, the phrase “clinical deterioration” refers to patients who experience an MG Crisis, which is defined as weakness from MG that is severe enough to necessitate intubation or to delay extubation following surgery, where the respiratory failure is due to weakness of respiratory muscles, severe bulbar (oropharyngeal) muscle weakness accompanies the respiratory muscle weakness, or is the predominant feature in a patient; or when there is significant symptomatic worsening to a score of 3 or a 2-point worsening from baseline on any one of the individual MG-Activities of Daily Living (MG-ADL) items other than double vision or eyelid droop; or administration of rescue therapy is provided to a patient whose, in the opinion of the investigator or investigator-designated physician, health would be in jeopardy, if rescue therapy were not given (e.g., emergent situations).

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method. Effective treatment may refer to, for example, alleviation of at least one symptom of MG.

The term “effective amount” refers to an amount of an agent that provides the desired biological, therapeutic and/or prophylactic result. That result can be reduction, amelioration, palliation, lessening, delaying and/or alleviation of one or more of the signs, symptoms, or causes of a disease, or any other desired alteration of a biological system. In one example, an “effective amount” is the amount of anti-C5 antibody or antigen binding fragment thereof useful, e.g., clinically proven, to alleviate at least one symptom of MG. An effective amount can be administered in one or more administrations.

As used herein, the terms “induction” and “induction phase” are used interchangeably and refer to the first phase of a dosing regimen.

As used herein, the terms “maintenance” and “maintenance phase” are used interchangeably and refer to the second phase of a dosing regimen. In some embodiments, treatment is continued as long as clinical benefit is observed or until unmanageable toxicity or disease progression occurs. The maintenance phase of ravulizumab dosing can last for between 6 weeks and the life of the subject. According to some embodiments, the maintenance phase lasts for 26-52, 26-78, 26-104, 26-130, 26-156, 26-182, 26-208 weeks, or more. In some embodiments, the maintenance phase lasts for greater than 26, 27, 28, 29, 30, 31, 32, 33, 34, 35, 36, 37, 38, 39, 40, 41, 42, 43, 44, 45, 46, 47, 48, 49, 50, 51, 52, 78, 104, 130, 156, or 182 weeks. According to some embodiments, the maintenance phase lasts for greater than 1, 2, 3, 4, 5, 10, 15, 20, 25, 30, 35, 40, 45, 50, 55, 60, 65, 70, 75, 80 years, or more years. In some embodiments, the maintenance phase lasts for the remainder of the subject's life.

In some embodiments, the ravulizumab multiphase dosing regimen includes a third phase. This third phase is used when an MG patient must undergo a rescue procedure to maintain clinical stability and includes administering plasma exchange/plasmapheresis (PE/PP) and/or dosing with IVIg. In this phase after plasma is exchanged, a dose of ravulizumab is administered to replace the drug lost during plasma exchange/plasmapheresis. According to some embodiments, supplemental study drug, e.g., ravulizumab, dosing is required if PE/PP or IVIg rescue therapy is provided on nondosing days. In another embodiment, if PE/PP or IVIg infusion is provided on a dosing day, it must occur prior to study drug administration. According to some embodiments, if PE/PP or IVIg is administered on nonscheduled dosing visits, patients receiving PE/PP are administered a supplemental dose 4 hours after the PE/PP session is completed. In another embodiment, patients receiving IVIg are administered a supplemental dose 4 hours after the last continuous session(s) of IVIg is completed. In some embodiments, supplemental dose amounts may or may not vary depending on PE/PP or IVIg (Table 1 and Table 2). In some embodiments, if PE/PP or IVIg is administered on scheduled dosing visits, regular dosing will be followed 60 minutes after the completion of PE/PP or VIg. In some embodiments, no gap is required between a supplemental dose and the regular scheduled dose.

TABLE 1
Supplemental dose when PE/PP is administered as rescue therapy on nonscheduled dosing visits

| | | | | Volume (mL) | | | |
|---|---|---|---|---|---|---|---|
| | | Body | Ravulizumab | | | Diluent | |
| | Ravulizumab or Placebo | Weight | Dose | | | (0.9% sodium | |
| Study Period | Dosing | (kg)¹ | (mg) | Ravulizumab | Placebo | chloride) | Total |

Ravulizumab Group

| | | | | | | | |
|---|---|---|---|---|---|---|---|
| Randominzed- | Loading dose | ≥40 to <60 | 1200 | 120 | 0 | 120 | 240 |
| Controlled | (Day 1) | ≥60 to <100 | 1500 | 150 | 0 | 150 | 300 |
| | | ≥100 | 1500 | 150 | 0 | 150 | 300 |
| | Maintenance dose | ≥40 to <60 | 1500 | 150 | 0 | 150 | 300 |
| | (Days 15, 71, 127) | ≥60 to <100 | 1800 | 180 | 0 | 180 | 360 |
| | | ≥100 | 1800 | 180 | 0 | 180 | 360 |
| Open-Label | Blinded dose² | ≥40 to <60 | 600 | 60 | 0 | 60 | 120 |
| Extension | (Day 183) | ≥60 to <100 | 600 | 60 | 0 | 60 | 120 |
| | | ≥100 | 600 | 60 | 0 | 60 | 120 |
| | Open-label maintenance dose | ≥40 to <60 | 1500 | 150 | 0 | 150 | 300 |
| | (Days 197 to 869 q8w) | ≥60 to <100 | 1800 | 180 | 0 | 180 | 360 |
| | | ≥100 | 1800 | 180 | 0 | 180 | 360 |

Placebo Group

| | | | | | | | |
|---|---|---|---|---|---|---|---|
| Randominzed- | Loading dose | ≥40 to <60 | 0 | 0 | 120 | 120 | 240 |
| Controlled | (Day 1) | ≥60 to <100 | 0 | 0 | 150 | 150 | 300 |
| | | ≥100 | 0 | 0 | 150 | 150 | 300 |
| | Maintenance dose | ≥40 to <60 | 0 | 0 | 150 | 150 | 300 |
| | (Days 15, 71, 127) | ≥60 to <100 | 0 | 0 | 180 | 180 | 360 |
| | | ≥100 | 0 | 0 | 180 | 180 | 360 |
| Open-Label | Blinded loading dose [symbol] | ≥40 to <60 | 600 | 60 | 0 | 60 | 120 |
| Extension | (Day 183) | ≥60 to <100 | 600 | 60 | 0 | 60 | 120 |
| | | ≥100 | 600 | 60 | 0 | 60 | 120 |
| | Open-label maintenance dose | ≥40 to <60 | 1500 | 150 | 0 | 150 | 300 |
| | (Days 197 to 869 q8w) | ≥60 to <100 | 1800 | 180 | 0 | 180 | 360 |
| | | ≥100 | 1800 | 180 | 0 | 180 | 360 |

¹Dose regimen will be based on the patient's most recently recorded body weight from a previous study/screening visit.
²Blinded dose on Day 183 (Week 26) for patients who were randomized to the ravulizumab group and are entering into the Open-Label Extension Period.
³Blinded loading dose on Day 183 (Week 26) for patients who were randomized to the placebo group and are entering into the Open-Label Extension Period. [symbol]
indicates data missing or illegible when filed

TABLE 2
Supplemental dose when intravenous immunoglobulin is administered as rescue therapy on nonscheduled dosing visits.

| | | | | Volume (mL) | | | |
|---|---|---|---|---|---|---|---|
| | | Body | Ravulizumab | | | Diluent | |
| | Ravulizumab or Placebo | Weight | Dose | | | (0.9% sodium | |
| Study Period | Dosing | (kg)¹ | (mg) | Ravulizumab | Placebo | chloride) | Total |

Ravulizumab Group

| | | | | | | | |
|---|---|---|---|---|---|---|---|
| Randominzed- | Loading dose | ≥40 to <60 | 600 | 60 | 0 | 60 | 120 |
| Controlled | (Day 1) | ≥60 to <100 | 600 | 60 | 0 | 60 | 120 |
| | | ≥100 | 600 | 60 | 0 | 60 | 120 |
| | Maintenance dose | ≥40 to <60 | 600 | 60 | 0 | 60 | 120 |
| | (Days 15, 71, 127) | ≥60 to <100 | 600 | 60 | 0 | 60 | 120 |
| | | ≥100 | 600 | 60 | 0 | 60 | 120 |
| Open-Label | Blinded dose² | ≥40 to <60 | 600 | 60 | 0 | 60 | 120 |
| Extension | (Day 183) | ≥60 to <100 | 600 | 60 | 0 | 60 | 120 |
| | | ≥100 | 600 | 60 | 0 | 60 | 120 |
| | Open-label maintenance dose | ≥40 to <60 | 600 | 60 | 0 | 60 | 120 |
| | (Days 197 to 869 q8w) | ≥60 to <100 | 600 | 60 | 0 | 60 | 120 |
| | | ≥100 | 600 | 60 | 0 | 60 | 120 |

Placebo Group

| | | | | | | | |
|---|---|---|---|---|---|---|---|
| Randominzed- | Loading dose | ≥40 to <60 | 0 | 0 | 60 | 60 | 120 |
| Controlled | (Day 1) | ≥60 to <100 | 0 | 0 | 60 | 60 | 120 |
| | | ≥100 | 0 | 0 | 60 | 60 | 120 |
| | Maintenance dose | ≥40 to <60 | 0 | 0 | 60 | 60 | 120 |
| | (Days 15, 71, 127) | ≥60 to <100 | 0 | 0 | 60 | 60 | 120 |
| | | ≥100 | 0 | 0 | 60 | 60 | 120 |
| Open-Label | Blinded loading dose [symbol] | ≥40 to <60 | 600 | 60 | 0 | 60 | 120 |
| Extension | (Day 183) | ≥60 to <100 | 600 | 60 | 0 | 60 | 120 |
| | | ≥100 | 600 | 60 | 0 | 60 | 120 |
| | Open-label maintenance dose | ≥40 to <60 | 600 | 60 | 0 | 60 | 120 |
| | (Days 197 to 869 q8w) | ≥60 to <100 | 600 | 60 | 0 | 60 | 120 |
| | | ≥100 | 600 | 60 | 0 | 60 | 120 |

¹Dose regimen will be based on the patient's most recently recorded body weight from a previous study/screening visit.
²Blinded dose on Day 183 (Week 26) for patients who were randomized to the ravulizumab group and are entering into the Open-Label Extension Period.
³Blinded loading dose on Day 183 (Week 26) for patients who were randomized to the placebo group and are entering into the Open-Label Extension Period. [symbol]
indicates data missing or illegible when filed

As used herein, the terms “loading dose” refers to the initial dose administered to the patient. Aloading may be, for example, 2400 mg, 2700 mg, or 3000 mg. Loading doses may be titered based on body weight.

As used herein, the terms “maintenance dose” or “maintenance phase” refers to adose administered to the patient after the loading dose. For example, amaintenance dose may be 3000 mg, 3300 mg, or 3600 mg. Maintenance doses may be titered based on body weight.

As used herein, the term “serum trough level” refers to the lowest concentration at which the agent (e.g., the anti-C5 antibody or antigen binding fragment thereof) or medicine is present in serum. In contrast, a“peak serum level” refers to the highest concentration of the agent in serum. The “average serum level” refers to the mean concentration of the agent in serum over time.

In one embodiment, the treatment regimens described are sufficient to maintain particular serum trough concentrations of the anti-C5 antibody or antigen binding fragment thereof. In one embodiment, for example, the treatment maintains a serum trough concentration of the anti-C5 antibody or antigen binding fragment thereof, of 50, 55, 60, 65, 70, 75, 80, 85, 90, 95, 100, 105, 110, 115, 120, 125, 130, 135, 140, 145, 150, 155, 160, 165, 170, 175, 180, 185, 190, 200, 205, 210, 215, 220, 225, 230, 240, 245, 250, 255, 260, 265, 270, 280, 290, 300, 305, 310, 315, 320, 325, 330, 335, 340, 345, 350, 355, 360, 365, 370, 375, 380, 385, 390, 395 or 400 μg/mL or greater. In one embodiment, the treatment maintains a serum trough concentration of the anti-C5 antibody or antigen binding fragment thereof of 100 μg/mL or greater. In another embodiment, the treatment maintains a serum trough concentration of the anti-C5 antibody or antigen binding fragment thereof of 150 μg/mL or greater. In another embodiment, the treatment maintains a serum trough concentration of the anti-C5 antibody or antigen binding fragment thereof of 200 μg/mL or greater. In another embodiment, the treatment maintains a serum trough concentration of the anti-C5 antibody or antigen binding fragment thereof of 250 μg/mL or greater. In another embodiment, the treatment maintains a serum trough concentration of the anti-C5 antibody or antigen binding fragment thereof of 300 μg/mL or greater. In another embodiment, the treatment maintains a serum trough concentration of the anti-C5 antibody or antigen binding fragment thereof of between 100 μg/mL and 200 μg/mL. In another embodiment, the treatment maintains a serum trough concentration of the anti-C5 antibody or antigen binding fragment thereof of about 175 μg/mL.

In another embodiment, to obtain an effective response, the anti-C5 antibody or antigen binding fragment thereof is administered to a patient in an amount and with a frequency to maintain a desired minimum free C5 concentration. In one embodiment, for example, the anti-C5 antibody or antigen binding fragment thereof is administered to the patient in an amount and with a frequency to maintain a free C5 concentration of 0.2 μg/mL, 0.3 μg/mL, 0.4 μg/mL, 0.5 μg/mL or less. In another embodiment, the anti-C5 antibody or antigen binding fragment thereof is administered to the patient in an amount and with a frequency to maintain a free C5 concentration of 0.309 to 0.5 μg/mL or less. In another embodiment, the treatment described herein reduces free C5 concentration by greater than 99% throughout the treatment period. In another embodiment, the treatment reduces free C5 concentration greater than 99.5% throughout the treatment period.

The term “antibody” describes polypeptides comprising at least one antibody derived antigen binding site (e.g., VH/VL region or Fv, or CDR). Antibodies include known forms of antibodies. The antibody can be, for example, a human antibody, a humanized antibody, a bispecific antibody, a chimeric antibody or a camelid antibody. The antibody also can be a Fab, Fab′2, scFv, SMIP, Affibody®, nanobody or a single domain antibody. The antibody also can be of any of the following isotypes: IgG1, IgG2, IgG3, IgG4, IgM, IgA1, IgA2, IgAsec, IgD, and IgE, and hybrid isotypes, e.g., IgG2/4. The antibody may be a naturally occurring antibody or may be an antibody that has been altered by a protein engineering technique (e.g., by mutation, deletion, substitution, conjugation to a non-antibody moiety). An antibody may include, for example. one or more variant amino acids (compared to a naturally occurring antibody), which changes a property (e.g., a functional property) of the antibody. Numerous such alterations are known in the art that affect, e.g., half-life, effector function, and/or immune responses to the antibody in a patient. The term antibody also includes artificial or engineered polypeptide constructs that comprise at least one antibody-derived antigen binding site.

### Anti-C5 Antibodies

The anti-C5 antibodies described herein bind to complement component C5 (e.g., human complement C5) and inhibit the cleavage of C5 into fragments C5a and C5b. Anti-C5 antibodies (or VH/VL domains or other antigen binding fragments derived therefrom) suitable for use herein can be generated using methods known in the art. Art-recognized anti-C5 antibodies can also be used. Antibodies that compete with any of these art-recognized antibodies for binding to C5 also can also be used.

Eculizumab (also known as Soliris®) is an anti-C5 antibody comprising heavy and light chains having sequences shown in SEQ ID NO: 10 and 11, respectively, or antigen binding fragments and variants thereof. Eculizumab is described in PCT/US2007/006606, the teachings of which are hereby incorporated by reference. In one embodiment the anti-C5 antibody, comprises the CDR1, CDR2 and CDR3 domains of the VH region of eculizumab having the sequence set forth in SEQ ID NO:7, and the CDR1, CDR2 and CDR3 domains of the VL region of eculizumab having the sequence set forth in SEQ ID NO:8. In another embodiment, the antibody comprises heavy chain CDR1, CDR2 and CDR3 domains having the sequences set forth in SEQ ID NOs: 1, 2 and 3, respectively, and light chain CDR1, CDR2 and CDR3 domains having the sequences set forth in SEQ ID NOs: 4, 5 and 6, respectively. In another embodiment, the antibody comprises VH and VL regions having the amino acid sequences set forth in SEQ ID NO:7 and SEQ ID NO:8, respectively.

Ravulizumab (also known as BNJ441, ALXN1210, or Ultomiris®) is an anti-C5 antibody comprising heavy and light chains having the sequences shown in SEQ ID NOs:14 and 11, respectively, or antigen binding fragments and variants thereof. Ravulizumab is described in PCT/US2015/019225 and U.S. Pat. No. 9,079,949, the teachings of which are hereby incorporated by reference. Ravulizumab selectively binds to human complement protein C5, inhibiting its cleavage to C5a and C5b during complement activation. This inhibition prevents the release of the proinflammatory mediator C5a and the formation of the cytolytic pore-forming membrane attack complex (MAC) C5b-9 while preserving the proximal or early components of complement activation (e.g., C3 and C3b) essential for the opsonization of microorganisms and clearance of immune complexes.

In one embodiment, the antibody comprises the heavy and light chain CDRs or variable regions of ravulizumab. Accordingly, in one embodiment, the antibody comprises the CDR1, CDR2 and CDR3 domains of the VH region of ravulizumab having the sequence set forth in SEQ ID NO:12, and the CDR1, CDR2 and CDR3 domains of the VL region of ravulizumab having the sequence set forth in SEQ ID NO:8. In another embodiment, the antibody comprises heavy chain CDR1, CDR2 and CDR3 domains having the sequences set forth in SEQ ID NOs:19, 18 and 3, respectively, and light chain CDR1, CDR2 and CDR3 domains having the sequences set forth in SEQ ID NOs:4, 5 and 6, respectively. In another embodiment, the antibody comprises VH and VL regions having the amino acid sequences set forth in SEQ ID NO:12 and SEQ ID NO:8, respectively.

Another exemplary anti-C5 antibody is antibody BNJ421 comprising heavy and light chains having the sequences shown in SEQ ID NOs:20 and 11, respectively, or antigen binding fragments and variants thereof. BNJ421 is described in PCT/US2015/019225 and U.S. Pat. No. 9,079,949, the entire teachings of which are hereby incorporated by reference.

In some embodiments, the antibody comprises the heavy and light chain CDRs or variable regions of BNJ421. Accordingly, in one embodiment, the antibody comprises the CDR1, CDR2 and CDR3 domains of the VH region of BNJ421 having the sequence set forth in SEQ ID NO:12, and the CDR1, CDR2 and CDR3 domains of the VL region of BNJ421 having the sequence set forth in SEQ ID NO:8. In another embodiment, the antibody comprises heavy chain CDR1, CDR2 and CDR3 domains having the sequences set forth in SEQ ID NOs:19, 18 and 3, respectively, and light chain CDR1, CDR2 and CDR3 domains having the sequences set forth in SEQ ID NOs:4, 5 and 6, respectively. In another embodiment, the antibody comprises VH and VL regions having the amino acid sequences set forth in SEQ ID NO:12 and SEQ ID NO:8, respectively.

The exact boundaries of CDRs have been defined differently according to different methods. In some embodiments, the positions of the CDRs or framework regions within a light or heavy chain variable domain can be as defined by Kabat et al. [(1991) “Sequences of Proteins of Immunological Interest.” NIH Publication No. 91-3242, U.S. Department of Health and Human Services, Bethesda, Md.]. In such cases, the CDRs can be referred to as “Kabat CDRs” (e.g., “Kabat LCDR2” or “Kabat HCDR1”). In some embodiments, the positions of the CDRs of a light or heavy chain variable region can be as defined by Chothia et al. (Nature, 342:877-83, 1989). Accordingly, these regions can be referred to as “Chothia CDRs” (e.g., “Chothia LCDR2” or “Chothia HCDR3”). In some embodiments, the positions of the CDRs of the light and heavy chain variable regions can be as defined by a Kabat-Chothia combined definition. In such embodiments, these regions can be referred to as “combined Kabat-Chothia CDRs” (Thomas, T. et al., Mol. Immunol., 33:1389-401, 1996).

Another exemplary anti-C5 antibody is the 7086 antibody described in U.S. Pat. Nos. 8,241,628 and 8,883,158. In one embodiment, the antibody comprises the heavy and light chain CDRs or variable regions of the 7086 antibody (see U.S. Pat. Nos. 8,241,628 and 8,883,158). In another embodiment, the antibody or antigen binding fragment thereof comprises heavy chain CDR1, CDR2 and CDR3 domains having the sequences set forth in SEQ ID NOs: 21, 22 and 23, respectively, and light chain CDR1, CDR2 and CDR3 domains having the sequences set forth in SEQ ID NOs: 24, 25 and 26, respectively. In another embodiment, the antibody or antigen binding fragment thereof comprises the VH region of the 7086 antibody having the sequence set forth in SEQ ID NO:27, and the VL region of the 7086 antibody having the sequence set forth in SEQ ID NO:28.

Another exemplary anti-C5 antibody is the 8110 antibody also described in U.S. Pat. Nos. 8,241,628 and 8,883,158. In one embodiment, the antibody comprises the heavy and light chain CDRs or variable regions of the 8110 antibody. In another embodiment, the antibody or antigen binding fragment thereof comprises heavy chain CDR1, CDR2 and CDR3 domains having the sequences set forth in SEQ ID NOs: 29, 30 and 31, respectively, and light chain CDR1, CDR2 and CDR3 domains having the sequences set forth in SEQ ID NOs: 32, 33 and 34, respectively. In another embodiment, the antibody comprises the VH region of the 8110 antibody having the sequence set forth in SEQ ID NO:35, and the VL region of the 8110 antibody having the sequence set forth in SEQ ID NO:36.

Another exemplary anti-C5 antibody is the 305LO5 antibody described in US2016/0176954A1. In one embodiment, the antibody comprises the heavy and light chain CDRs or variable regions of the 305LO5 antibody. In another embodiment, the antibody or antigen binding fragment thereof comprises heavy chain CDR1, CDR2 and CDR3 domains having the sequences set forth in SEQ ID NOs: 37, 38 and 39, respectively, and light chain CDR1, CDR2 and CDR3 domains having the sequences set forth in SEQ ID NOs: 40, 41 and 42, respectively. In another embodiment, the antibody comprises the VH region of the 305LO5 antibody having the sequence set forth in SEQ ID NO:43, and the VL region of the 305LO5 antibody having the sequence set forth in SEQ ID NO:4.

Another exemplary anti-C5 antibody is the SKY59 antibody (Fukuzawa T. et al., Sci. Rep., 7:1080, 2017). In one embodiment, the antibody comprises the heavy and light chain CDRs or variable regions of the SKY59 antibody. In another embodiment, the antibody or antigen binding fragment thereof comprises a heavy chain comprising SEQ ID NO:45 and a light chain comprising SEQ ID NO:46.

Another exemplary anti-C5 antibody is the H4H12166PP antibody described in PCT/US2017/037226 and US2017/0355757A1. In one embodiment, the antibody comprises the heavy and light chain CDRs or variable regions of the H4H12166PP antibody. In another embodiment, the antibody or antigen binding fragment thereof comprises the VH region of the H4H12166PP antibody having the sequence set forth in SEQ ID NO:47, and the VL region of the H4H12166PP antibody having the sequence set forth in SEQ ID NO48. In another embodiment, the antibody or antigen binding fragment thereof comprises a heavy chain comprising SEQ ID NO:49 and a light chain comprising SEQ ID NO:50.

In one embodiment, a patient is treated with eculizumab and then switched to treatment with the 7086 antibody, the 8110 antibody, the 305LO5 antibody, the SKY59 antibody, the H4H12166PP antibody or ravulizumab. In another embodiment, the patient is switched from an anti-C5 antibody (e.g., eculizumab, the 7086 antibody, the 8110 antibody, the 305LO5 antibody, the SKY59 antibody or the H4H12166PP antibody) to another anti-C5 antibody (e.g., ravulizumab) during the course of treatment. In a particular embodiment, the patient is switched from eculizumab to ravulizumab during the course of treatment.

In some embodiments, an anti-C5 antibody described herein comprises a heavy chain CDR1 comprising or consisting of the following amino acid sequence: GHIFSNYWIQ (SEQ ID NO:19). In some embodiments, an anti-C5 antibody described herein comprises a heavy chain CDR2 comprising or consisting of the following amino acid sequence: EILPGSGHTEYTENFKD (SEQ ID NO:18). In some embodiments, an anti-C5 antibody described herein comprises a heavy chain variable region comprising the following amino acid sequence:

(SEQ ID NO: 12)
QVQLVQSGAEVKKPGASVKVSCKASGHIFSNYWIQWVRQAPGQGLEWMGE
ILPGSGHTEYTENFKDRVTMTRDTSTSTVYMELSSLRSEDTAVYYCARYF
FGSSPNWYFDVWGQGTLVTVSS.

In some embodiments, an anti-C5 antibody described herein comprises a light chain PGP-23.DNA variable region comprising the following amino acid sequence:

(SEQ ID NO: 8)
DIQMTQSPSSLSASVGDRVTITCGASENIYGALNWYQQKPGKAPKLLIYG
ATNLADGVPSRFSGSGSGTDFTLTISSLQPEDFATYYCQNVLNTPLTFGQ
GTKVEIK.

An anti-C5 antibody described herein can, in some embodiments, comprise a variant human Fc constant region that binds to human neonatal Fc receptor (FcRn) with greater affinity than that of the native human Fc constant region from which the variant human Fc constant region was derived. The Fc constant region can comprise, for example, one or more (e.g., two, three, four, five, six, seven or eight or more) amino acid substitutions relative to the native human Fc constant region from which the variant human Fc constant region was derived. The substitutions can increase the binding affinity of an IgG antibody containing the variant Fc constant region to FcRn at pH 6.0, while maintaining the pH dependence of the interaction. Methods for testing whether one or more substitutions in the Fc constant region of an antibody increase the affinity of the Fc constant region for FcRn at pH 6.0 (while maintaining pH dependence of the interaction) are known in the art and exemplified in the working examples (see, e.g., PCT/US2015/019225 and U.S. Pat. No. 9,079,949 the disclosures of each of which are incorporated herein by reference in their entirety).

Substitutions that enhance the binding affinity of an antibody Fc constant region for FcRn are known in the art and include, e.g., (1) the M252Y/S254T/T256E triple substitution (Dall'Acqua, W. et al., J. Biol. Chem., 281:23514-24, 2006); (2) M428L or T250Q/M428L substitutions (Hinton, P. et al., J. Biol. Chem., 279:6213-6, 2004; Hinton, P. et al., J. Immumol., 176:346-56, 2006); and (3) N434A or T307/E380A/N434A substitutions (Petkova, S. et al., Int. Immunol., 18:1759-69, 2006). Additional substitution pairings, e.g., P257I/Q31I, P257I/N434H, and D376V/N434H (Datta-Mannan, A. et al., J. Biol. Chem., 282:1709-17, 2007) are also contemplated herein.

In some embodiments, the variant constant region has a substitution at EU amino acid residue 255 for valine. In some embodiments, the variant constant region has a substitution at EU amino acid residue 309 for asparagine. In some embodiments, the variant constant region has a substitution at EU amino acid residue 312 for isoleucine. In some embodiments, the variant constant region has a substitution at EU amino acid residue 386.

In some embodiments, the variant Fc constant region comprises no more than 30 (e.g., no more than 29, 28, 27, 26, 25, 24, 23, 22, 21, 20, 19, 18, 17, 16, 15, 14, 13, 12, 11, 10, 9, 8, 7, 6, 5, 4, 3 or 2) amino acid substitutions, insertions or deletions relative to the native constant region from which it was derived. In some embodiments, the variant Fc constant region comprises one or more amino acid substitutions selected from the group consisting of: M252Y, S254T, T256E, N434S, M428L, V259I, T250I and V308F. In some embodiments, the variant human Fc constant region comprises a methionine at position 428 and an asparagine at position 434, each in EU numbering. In some embodiments, the variant Fc constant region comprises a 428I/434S double substitution as described in, e.g., U.S. Pat. No. 8,088,376.

In some embodiments the precise location of these mutations may be shifted from the native human Fc constant region position due to antibody engineering. The 428L/434S double substitution when used in a IgG2/4 chimeric Fc, for example, may correspond to 429L and 435S as in the M429L and N435S variants found in BNJ441 (ravulizumab) and described in U.S. Pat. No. 9,079,949, the disclosure of which is incorporated herein by reference in its entirety.

In some embodiments, the variant constant region comprises a substitution at amino acid position 237, 238, 239, 248, 250, 252, 254, 255, 256, 257, 258, 265, 270, 286, 289, 297, 298, 303, 305, 307, 308, 309, 311, 312, 314, 315, 317, 325, 332, 334, 360, 376, 380, 382, 384, 385, 386, 387, 389, 424, 428, 433, 434 or 436 (EU numbering) relative to the native human Fc constant region. In some embodiments, the substitution is selected from the group consisting of: methionine for glycine at position 237; alanine for proline at position 238; lysine for serine at position 239; isoleucine for lysine at position 248; alanine, phenylalanine, isoleucine, methionine, glutamine, serine, valine, tryptophan, or tyrosine for threonine at position 250; phenylalanine, tryptophan, or tyrosine for methionine at position 252; threonine for serine at position 254; glutamic acid for arginine at position 255; aspartic acid, glutamic acid, or glutamine for threonine at position 256; alanine, glycine, isoleucine, leucine, methionine, asparagine, serine, threonine, or valine for proline at position 257; histidine for glutamic acid at position 258; alanine for aspartic acid at position 265; phenylalanine for aspartic acid at position 270; alanine, or glutamic acid for asparagine at position 286; histidine for threonine at position 289; alanine for asparagine at position 297; glycine for serine at position 298; alanine for valine at position 303; alanine for valine at position 305; alanine, aspartic acid, phenylalanine, glycine, histidine, isoleucine, lysine, leucine, methionine, asparagine, proline, glutamine, arginine, serine, valine, tryptophan, or tyrosine for threonine at position 307; alanine, phenylalanine, isoleucine, leucine, methionine, proline, glutamine, or threonine for valine at position 308; alanine, aspartic acid, glutamic acid, proline, or arginine for leucine or valine at position 309; alanine, histidine, or isoleucine for glutamine at position 311; alanine or histidine for aspartic acid at position 312; lysine or arginine for leucine at position 314; alanine or histidine for asparagine at position 315; alanine for lysine at position 317; glycine for asparagine at position 325; valine for isoleucine at position 332; leucine for lysine at position 334; histidine for lysine at position 360; alanine for aspartic acid at position 376; alanine for glutamic acid at position 380; alanine for glutamic acid at position 382; alanine for asparagine or serine at position 384; aspartic acid or histidine for glycine at position 385; proline for glutamine at position 386; glutamic acid for proline at position 387; alanine or serine for asparagine at position 389; alanine for serine at position 424; alanine, aspartic acid, phenylalanine, glycine, histidine, isoleucine, lysine, leucine, asparagine, proline, glutamine, serine, threonine, valine, tryptophan, or tyrosine for methionine at position 428; lysine for histidine at position 433; alanine, phenylalanine, histidine, serine, tryptophan, or tyrosine for asparagine at position 434; and histidine for tyrosine or phenylalanine at position 436, all in EU numbering.

Suitable anti-C5 antibodies for use in the methods described herein can comprise a heavy chain polypeptide comprising the amino acid sequence of SEQ ID NO:14 and/or a light chain polypeptide comprising the amino acid sequence of SEQ ID NO:11. Alternatively, the anti-C5 antibodies for use in the methods described herein can comprise a heavy chain polypeptide comprising the amino acid sequence of SEQ ID NO:20 and/or a light chain polypeptide comprising the amino acid sequence of SEQ ID NO:11.

In one embodiment, the antibody binds to C5 at pH 7.4 and 25° C. (and, otherwise, under physiologic conditions) with an affinity dissociation constant (K_{D}) that is at least 0.1 (e.g., at least 0.15, 0.175, 0.2, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95 or 0.975) nM. In some embodiments, the K_{D} of the anti-C5 antibody or antigen binding fragment thereof is no greater than 1 (e.g., no greater than 0.9, 0.8, 0.7, 0.6, 0.5, 0.4, 0.3 or 0.2) nM.

In some embodiments, the [(K_{D} of the antibody for C5 at pH 6.0 at 25° C.)/(K_{D} of the antibody for C5 at pH 7.4 at 25° C.)] is greater than 21 (e.g., greater than 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, 400, 450, 500, 600, 700, 800, 900, 1000, 1500, 2000, 2500, 3000, 3500, 4000, 4500, 5000, 5500, 6000, 6500, 7000, 7500 or 8000).

Methods for determining whether an antibody binds to a protein antigen and/or the affinity for an antibody to a protein antigen are known in the art. The binding of an antibody to a protein antigen, for example, can be detected and/or quantified using a variety of techniques such as, but not limited to, Western blot, dot blot, surface plasmon resonance (SPR) method (e.g., BIAcore system: Pharmacia Biosensor AB, Uppsala, Sweden and Piscataway, N.J.), or enzyme-linked immunosorbent assay (ELISA) (see, e.g., Benny K. C. Lo (2004) “Antibody Engineering: Methods and Protocols,” Humana Press (ISBN: 1588290921); Johne, B. et al., J. Immunol. Meth., 160:191-8, 1993; Jönsson, U. et al., Ann. Biol. Cli., 51:19-26, 1993; Jönsson, U. et al., Biotechniques, 11:620-7, 1991). Additional methods for measuring, for example, affinity (e.g. dissociation and association constants) are set forth in the working examples.

As used herein, the term “kₐ” refers to the rate constant for association of an antibody to an antigen. The term “k_{d}” refers to the rate constant for dissociation of an antibody from the antibody/antigen complex. And the term “K_{D}” refers to the equilibrium dissociation constant of an antibody-antigen interaction. The equilibrium dissociation constant is deduced from the ratio of the kinetic rate constants, K_{D}=kₐ/k_{d}. Such determinations preferably are measured at 25° C. or 37° C. The kinetics of antibody binding to human C5 can be determined, for example, at pH 8.0, 7.4, 7.0, 6.5 and 6.0 via surface plasmon resonance (SPR) on a BIAcore 3000 instrument using an anti-Fc capture method to immobilize the antibody.

Methods for determining whether a particular antibody described herein inhibits C5 cleavage are known in the art. Inhibition of human complement component C5 can reduce the cell-lysing ability of complement in a subject's body fluids. Such reductions of the cell-lysing ability of complement present in the body fluid(s) can be measured by methods known in the art such as, for example, by a conventional hemolytic assay such as the hemolysis assay described by Kabat and Mayer (eds.), “Experimental Immunochemistry, 2^{nd} Edition,” 135-240, Springfield, Ill., CC Thomas (1961), pages 135-139, or a conventional variation of that assay such as the chicken erythrocyte hemolysis method (Hillmen, P. et al., N. Engl. J. Med., 350:552-9, 2004). Methods for determining whether a candidate compound inhibits the cleavage of human C5 into forms C5a and C5b are known in the art (Evans, M. et al., Mol. Immunol., 32:1183-95, 1995). The concentration and/or physiologic activity of C5a and C5b in a body fluid can be measured, for example, by methods known in the art. For C5b, hemolytic assays or assays for soluble C5b-9 as discussed herein can be used. Other assays known in the art can also be used. Using these or other suitable assays, candidate agents capable of inhibiting human complement component C5 can be screened.

Immunological techniques such as, but not limited to, ELISA can be used to measure the protein concentration of C5 and/or its split products to determine the ability of an anti-C5 antibody or antigen binding fragment thereof to inhibit conversion of C5 into biologically active products. In some embodiments, C5a generation is measured. In some embodiments, C5b-9 neoepitope-specific antibodies are used to detect the formation of terminal complement.

Hemolytic assays can be used to determine the inhibitory activity of an anti-C5 antibody or antigen binding fragment thereof on complement activation. To determine the effect of an anti-C5 antibody or antigen binding fragment thereof on classical complement pathway-mediated hemolysis in a serum test solution in vitro, for example, sheep erythrocytes coated with hemolysin or chicken erythrocytes sensitized with anti-chicken erythrocyte antibody are used as target cells. The percentage of lysis is normalized by considering 100% lysis equal to the lysis occurring in the absence of the inhibitor. In some embodiments, the classical complement pathway is activated by a human IgM antibody, for example, as utilized in the Wieslab® Classical Pathway Complement Kit (Wieslab® COMPL CP310, Euro-Diagnostica, Sweden). Briefly, the test serum is incubated with an anti-C5 antibody or antigen binding fragment thereof in the presence of a human IgM antibody. The amount of C5b-9 that is generated is measured by contacting the mixture with an enzyme conjugated anti-C5b-9 antibody and a fluorogenic substrate and measuring the absorbance at the appropriate wavelength. As a control, the test serum is incubated in the absence of the anti-C5 antibody or antigen binding fragment thereof. In some embodiments, the test serum is a C5-deficient serum reconstituted with a C5 polypeptide.

To determine the effect of an anti-C5 antibody or antigen binding fragment thereof on alternative pathway-mediated hemolysis, unsensitized rabbit or guinea pig erythrocytes can be used as the target cells. In some embodiments, the serum test solution is a C5-deficient serum reconstituted with a C5 polypeptide. The percentage of lysis is normalized by considering 100% lysis equal to the lysis occurring in the absence of the inhibitor. In some embodiments, the alternative complement pathway is activated by lipopolysaccharide molecules, for example, as utilized in the Wieslab® Alternative Pathway Complement Kit (Wieslab® COMPL AP330, Euro-Diagnostica, Sweden). Briefly, the test serum is incubated with an anti-C5 antibody or antigen binding fragment thereof in the presence of lipopolysaccharide. The amount of C5b-9 that is generated is measured by contacting the mixture with an enzyme conjugated anti-C5b-9 antibody and a fluorogenic substrate and measuring the fluorescence at the appropriate wavelength. As a control, the test serum is incubated in the absence of the anti-C5 antibody or antigen binding fragment thereof.

In some embodiments, C5 activity, or inhibition thereof, is quantified using a CH50eq assay. The CH50eq assay is a method for measuring the total classical complement activity in serum. This test is a lytic assay that uses antibody-sensitized erythrocytes as the activator of the classical complement pathway and various dilutions of the test serum to determine the amount required to give 50% lysis (CH50). The percent hemolysis can be determined, for example, using a spectrophotometer. The CH50eq assay provides an indirect measure of terminal complement complex (TCC) formation, since the TCC themselves are directly responsible for the hemolysis that is measured. Briefly, to activate the classical complement pathway, undiluted serum samples (e.g., reconstituted human serum samples) are added to microassay wells containing the antibody-sensitized erythrocytes to thereby generate TCC. Next, the activated serum samples are diluted in microassay wells, which are coated with a capture reagent (e.g., an antibody that binds to one or more components of the TCC). The TCC present in the activated samples bind to the monoclonal antibodies coating the surface of the microassay wells. The wells are washed and to each well is added a detection reagent that is detectably labeled and recognizes the bound TCC. The detectable label can be, e.g., a fluorescent label or an enzymatic label. The assay results are expressed in CH50 unit equivalents per milliliter (CH50 U Eq/mL).

Inhibition, e.g., as it pertains to terminal complement activity, includes at least a 5 (e.g., at least a 6, 7, 8, 9, 10, 15, 20, 25, 30, 35, 40, 45, 50, 55 or 60)% decrease in the activity of terminal complement in, e.g., a hemolytic assay or CH50eq assay as compared to the effect of a control antibody (or antigen-binding fragment thereof) under similar conditions and at an equimolar concentration. Substantial inhibition, as used herein, refers to inhibition of a given activity (e.g., terminal complement activity) of at least 40 (e.g., at least 45, 50, 55, 60, 65, 70, 75, 80, 85, 90 or 95 or greater) %. In some embodiments, an anti-C5 antibody described herein contains one or more amino acid substitutions relative to the CDRs of eculizumab (i.e., SEQ ID NOs:1-6), yet retains at least 30 (e.g., at least 31, 32, 33, 34, 35, 36, 37, 38, 39, 40, 41, 42, 43, 44, 45, 46, 47, 48, 49, 50, 55, 60, 65, 70, 75, 80, 85, 90 or 95) oof the complement inhibitory activity of eculizumab in a hemolytic assay or CH50eq assay.

An anti-C5 antibody described herein has a serum half-life in humans that is at least 20 (e.g., at least 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54 or 55) days. In another embodiment, the anti-C5 antibody described herein has a serum half-life in humans that is at least 40 days. In another embodiment, the anti-C5 antibody described herein has a serum half-life in humans that is approximately 43 days. In another embodiment, the anti-C5 antibody described herein has a serum half-life in humans that is between 39-48 days. Methods for measuring the serum half-life of an antibody are known in the art. In some embodiments, an anti-C5 antibody or antigen binding fragment thereof described herein has a serum half-life that is at least 20 (e.g., at least 30, 35, 40, 45, 50, 55, 60, 65, 70, 75, 80, 85, 90, 95, 100, 125, 150, 175, 200, 250, 300, 400, 500) % greater than the serum half-life of eculizumab, e.g., as measured in one of the mouse model systems described in the working examples (e.g., the C5-deficient/NOD/scid mouse or hFcRn transgenic mouse model system).

In one embodiment, the antibody competes for binding with, and/or binds to the same epitope on C5 as an antibody described herein. The term “binds to the same epitope” with reference to two or more antibodies means that the antibodies bind to the same segment of amino acid residues, as determined by a given method. Techniques for determining whether antibodies bind to the “same epitope on C5” with the antibodies described herein include, for example, epitope mapping methods, such as, x-ray analyses of crystals of antigen:antibody complexes that provides atomic resolution of the epitope and hydrogen/deuterium exchange mass spectrometry (HDX-MS). Other methods monitor the binding of the antibody to peptide antigen fragments or mutated variations of the antigen where loss of binding due to a modification of an amino acid residue within the antigen sequence is often considered an indication of an epitope component. Computational combinatorial methods for epitope mapping can also be used. These methods rely on the ability of the antibody of interest to affinity isolate specific short peptides from combinatorial phage display peptide libraries. Antibodies having the same VH and VL or the same CDR1, 2 and 3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, can be determined using known competition experiments. In some embodiments, an antibody competes with and inhibits binding of another antibody to a target by at least 10%, 20%, 30%, 40%, 50%, 60%, 70%, 80%, 90% or 100%. The level of inhibition or competition may be different depending on which antibody is the “blocking antibody” (i.e., the cold antibody that is incubated first with the target). Competing antibodies can bind, for example, to the same epitope, an overlapping epitope or to adjacent epitopes (e.g., as evidenced by steric hindrance).

Anti-C5 antibodies or antigen-binding fragments thereof described herein, used in the methods described herein, can be generated using a variety of art-recognized techniques. Monoclonal antibodies may be obtained by various techniques familiar to those skilled in the art. Briefly, spleen cells from an animal immunized with a desired antigen are immortalized, commonly by fusion with a myeloma cell (Köhler, G. & Milstein, C., Eur. J. Immunol., 6:511-9, 1976). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. One can alternatively isolate DNA sequences that encode a monoclonal antibody or a binding fragment thereof by screening a DNA library from human B cells (Huse, W. et al., Science, 246:1275-81, 1989).

### Compositions

Pharmaceutical compositions comprising ravulizumab, either alone or in combination with prophylactic agents, therapeutic agents, and/or pharmaceutically acceptable carriers are provided. The pharmaceutical compositions comprising ravulizumab provided herein are for use in, for example, diagnosing, detecting or monitoring a disorder, in preventing, treating, managing or ameliorating a disorder or one or more symptoms thereof, and/or in research. Formulations of pharmaceutical compositions, either alone or in combination with prophylactic agents, therapeutic agents, and/or pharmaceutically acceptable carriers, are known in the art.

Also, provided herein are compositions comprising an anti-C5 antibody or antigen binding fragment thereof for use in the treatment methods described herein, wherein a patient is switched from one anti-C5 antibody (e.g., eculizumab) to another anti-C5 antibody (e.g., ravulizumab) during the course of treatment.

The composition can be formulated as a pharmaceutical solution, e.g., for administration to a subject for the treatment or prevention of MG. The pharmaceutical composition can include a pharmaceutically acceptable carrier. As used herein, a “pharmaceutically acceptable carrier” refers to, and includes, any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt, sugars, carbohydrates, polyols and/or tonicity modifiers.

The composition can be formulated according to known methods (Gennaro (2000) “Remington: The Science and Practice of Pharmacy,” 20^{th} Edition, Lippincott, Williams & Wilkins (ISBN: 0683306472); Ansel et al. (1999) “Pharmaceutical Dosage Forms and Drug Delivery Systems,” 7′ Edition, Lippincott Williams & Wilkins Publishers (ISBN: 0683305727); and Kibbe (2000) “Handbook of Pharmaceutical Excipients American Pharmaceutical Association,” 3^{rd} Edition (ISBN: 091733096X)). In some embodiments, a composition can be formulated, for example, as a buffered solution at a suitable concentration and suitable for storage at 2-8° C. (e.g., 4° C.). In some embodiments, a composition can be formulated for storage at a temperature below 0° C. (e.g., −20° C. or −80° C.). In some embodiments, the composition can be formulated for storage for up to 2 years (e.g., 1 month, 2 months, 3 months, 4 months, 5 months, 6 months, 7 months, 8 months, 9 months, 10 months, 11 months, 1 year, 1½ years or 2 years) at 2-8° C. (e.g., 4° C.). Thus, in some embodiments, the compositions described herein are stable in storage for at least 1 year at 2-8° C. (e.g., 4° C.).

The pharmaceutical compositions can be in a variety of forms. These forms include, e.g., liquid, semi-solid and solid dosage forms, such as liquid solutions (e.g., injectable and infusible solutions), dispersions or suspensions, tablets, pills, powders, liposomes and suppositories. The preferred form depends, in part, on the intended mode of administration and therapeutic application. Compositions containing a composition intended for systemic or local delivery can, for example, be in the form of injectable or infusible solutions. The compositions can be formulated for administration by a parenteral mode (e.g., intravenous, subcutaneous, intraperitoneal, or intramuscular injection). “Parenteral administration,” “administered parenterally” and other grammatically equivalent phrases, as used herein, refer to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrastemal injection and infusion. In one embodiment, the antibodies are formulated for intravenous administration.

An exemplary, non-limiting range for a therapeutically or prophylactically effective amount of ravulizumab or other anti-C5 antibodies such as eculizumab, BNJ 421, 7086, 8110, SKY59 and H4H12166PP provided herein is 600-5000 mg, for example, 900-2000 mg. It is to be noted that dosage values may vary with the type and severity of the condition to be alleviated. It is to be further understood that for any particular subject, specific dosage regimens may be adjusted over time according to the individual need and the professional judgment of the person administering or supervising the administration of the compositions, and that dosage ranges set forth herein are exemplary only and are not intended to limit the scope or practice of the claimed methods.

### Combination Therapy

An anti-C5 antibody provided herein also can be administered with one or more additional medicaments or therapeutic agents useful in the treatment of MG. The additional agent can be, for example, a therapeutic agent art-recognized as being useful to treat MG. The combination can also include more than one additional agents, e.g., two or three additional agents. The binding agent in various embodiments is administered with an agent that is a protein, a peptide, a carbohydrate, a drug, a small molecule, or a genetic material (e.g., DNA or RNA). In various embodiments, the agent is one or more cholinesterase inhibitors, one or more corticosteroids, and/or one or more immunosuppressive drugs (most commonly azathioprine [AZA], cyclosporin, and/or mycophenolate mofetil [MMF]).

### Methods

Provided herein are methods for treating complement-associated disorder(s) (e.g., MG, e.g., gMG, e.g., gMG when the patient is anti-AChR antibody positive) in a human patient, comprising administering to the patient an anti-C5 antibody or antigen binding fragment thereof wherein the anti-C5 antibody or antigen binding fragment thereof is administered (or is for administration) according to a particular clinical dosage regimen (i.e., at a particular dose amount and according to a specific dosing schedule).

In some embodiments, MG includes gMG. In some embodiments, gMG is characterized as including subjects or patients positive for auto-antibodies binding to AChR who continue to show marked generalized weakness or bulbar signs and symptoms of MG while receiving current standard of care for MG such as cholinesterase inhibitor therapy and IST or who require chronic plasma exchange or chronic IVIg to maintain clinical stability.

In one embodiment, the anti-C5 antibody or antigen binding fragment thereof is administered once on Day 1 of the administration cycle, once on Day 15 of the administration cycle, and every eight weeks thereafter. In one embodiment, the anti-C5 antibody or antigen binding fragment thereof is administered every eight weeks after the administration cycle for an extension period up to two years (e.g., at a dose of 3000 mg, 3300 mg or 3600 mg).

In another embodiment, the anti-C5 antibody or antigen binding fragment thereof is administered for one or more administration cycles. In one embodiment, the administration cycle is 26 weeks. In another embodiment, the treatment comprises at least 1, 2, 3, 4, 5, 6, 7, 8, 9, 10 or 11 cycles. In another embodiment, the treatment is continued for the lifetime of the human patient.

In another embodiment, a patient switches from receiving one C5 inhibitor to a different C5 inhibitor during the course of treatment. Different anti-C5 antibodies can be administered during separate treatment periods. In one embodiment, for example, a method of treating a human patient having a complement-associated disorder (e.g., MG) who is being treated with eculizumab is provided, the method comprising discontinuing treatment with eculizumab and switching the patient to treatment with an alternative complement inhibitor. In another embodiment, a method of treating a human patient having a complement-associated disorder who is being treated with ravulizumab is provided, the method comprising discontinuing treatment with ravulizumab and switching the patient to treatment with an alternative complement inhibitor.

Exemplary alternative complement inhibitors include, but are not limited to antibodies or antigen binding fragments thereof, small molecules, polypeptides, polypeptide analogs, peptidomimetics, siRNA and aptamers. In one embodiment, the alternative complement inhibitor inhibits one or more of complement components C1, C2, C3, C4, C5, C6, C7, C8, C9, Factor D, Factor B, properdin, MBL, MASP-1, MASP-2, or biologically active fragments thereof. In another embodiment, the alternative complement inhibitor inhibits the anaphylatoxic activity associated with C5a and/or the assembly of the membrane attack complex associated with C5b. In another embodiment, the alternative complement inhibitor is selected from the group consisting of CR1, LEX-CR1, MCP, DAF, CD59, Factor H, cobra venom factor, FUT-175, complestatin and K76 COOH.

Exemplary alternative anti-C5 antibodies included, but are not limited to, (i) eculizumab, (ii), an antibody or antigen binding fragment thereof comprising heavy chain CDR1, CDR2 and CDR3 domains comprising SEQ ID NOs: 21, 22 and 23, respectively, and light chain CDR1, CDR2 and CDR3 domains comprising SEQ ID NOs: 24, 25 and 26, respectively, (iii) an antibody or antigen binding fragment thereof comprising a heavy chain variable region comprising SEQ ID NO:27 and a light chain variable region comprising SEQ ID NO:28, (iv) an antibody or antigen binding fragment thereof comprising heavy chain CDR1, CDR2 and CDR3 domains comprising SEQ ID NOs: 29, 30 and 31, respectively, and light chain CDR1, CDR2 and CDR3 domains comprising SEQ ID NOs: 32, 33 and 34, respectively, (v) an antibody or antigen binding fragment thereof comprising a heavy chain variable region comprising SEQ ID NO:35 and a light chain variable region comprising SEQ ID NO:36, (vi) an antibody or antigen binding fragment thereof comprising heavy chain CDR1, CDR2 and CDR3 domains comprising SEQ ID NOs: 37, 38 and 39, respectively, and light chain CDR1, CDR2 and CDR3 domains comprising SEQ ID NOs: 40, 41 and 42, respectively, (vii) an antibody or antigen binding fragment thereof comprising a heavy chain variable region comprising SEQ ID NO:43 and a light chain variable region comprising SEQ ID NO:44, and (viii) an antibody or antigen binding fragment thereof comprising a heavy chain comprising SEQ ID NO:45 and a light chain comprising SEQ ID NO:46.

In another embodiment, the patient is treated with ravulizumab and then switched to treatment with the 7086 antibody, the 8110 antibody, the 305L05 antibody, the SKY59 antibody, the H4H12166PP antibody or eculizumab. In another embodiment, the patient is switched from an anti-C5 antibody (e.g., eculizumab, the 7086 antibody, the 8110 antibody, the 305LO5 antibody, the SKY59 antibody or the H4H12166PP antibody) to another anti-C5 antibody (e.g., ravulizumab) during the course of treatment. In a particular embodiment, the patient is switched from eculizumab to ravulizumab during the course of treatment.

In one embodiment, the anti-C5 antibody is administered (or is for administration) according to a particular clinical dosage regimen (e.g., at a particular dose amount and/or according to a specific dosing schedule). In one embodiment, the anti-C5 antibody is administered at a fixed dose that is fixed irrespective of the weight of the patient. As used herein, the terms “fixed dose,” “flat dose” and “flat-fixed dose” are used interchangeably and refer to a dose that is administered to a patient without regard for the weight or body surface area (BSA) of the patient. The fixed or flat dose is therefore, not provided as a mg/kg dose, but rather as an absolute amount of the anti-C5 antibody or antigen binding fragment thereof.

In one embodiment, the anti-C5 antibody is administered at a fixed dose of 10 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, 6100 mg, 6200 mg, 6300 mg, 6400 mg, 6500 mg, 6600 mg, 6700 mg, 6800 mg, 6900 mg, 7000 mg, 7100 mg, 7200 mg, 7300 mg, 7400 mg, 7500 mg, 7600 mg, 7700 mg, 7800 mg, 7900 mg, 8000 mg, 8100 mg, 8200 mg, 8300 mg, 8400 mg, 8500 mg, 8600 mg, 8700 mg, 8800 mg, 8900 mg, 9000 mg, 9100 mg, 9200 mg, 9300 mg, 9400 mg, 9500 mg, 9600 mg, 9700 mg, 9800 mg, 9900 mg, 10000 mg, 10100 mg, 10200 mg, 10300 mg, 10400 mg, 10500 mg, 10600 mg, 10700 mg, 10800 mg, 10900 mg or 11000 mg, without regard to the patient's weight.

In another embodiment, the dose of the anti-C5 antibody is based on the weight of the patient. In one embodiment, 10 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, 6100 mg, 6200 mg, 6300 mg, 6400 mg, 6500 mg, 6600 mg, 6700 mg, 6800 mg, 6900 mg, 7000 mg, 7100 mg, 7200 mg, 7300 mg, 7400 mg, 7500 mg, 7600 mg, 7700 mg, 7800 mg, 7900 mg, 8000 mg, 8100 mg, 8200 mg, 8300 mg, 8400 mg, 8500 mg, 8600 mg, 8700 mg, 8800 mg, 8900 mg, 9000 mg, 9100 mg, 9200 mg, 9300 mg, 9400 mg, 9500 mg, 9600 mg, 9700 mg, 9800 mg, 9900 mg, 10000 mg, 10100 mg, 10200 mg, 10300 mg, 10400 mg, 10500 mg, 10600 mg, 10700 mg, 10800 mg, 10900 mg or 11000 mg of the anti-C5 antibody or antigen binding fragment thereof is administered to a patient weighing ≥40 to <60 kg.

In another embodiment, 10 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, 6100 mg, 6200 mg, 6300 mg, 6400 mg, 6500 mg, 6600 mg, 6700 mg, 6800 mg, 6900 mg, 7000 mg, 7100 mg, 7200 mg, 7300 mg, 7400 mg, 7500 mg, 7600 mg, 7700 mg, 7800 mg, 7900 mg, 8000 mg, 8100 mg, 8200 mg, 8300 mg, 8400 mg, 8500 mg, 8600 mg, 8700 mg, 8800 mg, 8900 mg, 9000 mg, 9100 mg, 9200 mg, 9300 mg, 9400 mg, 9500 mg, 9600 mg, 9700 mg, 9800 mg, 9900 mg, 10000 mg, 10100 mg, 10200 mg, 10300 mg, 10400 mg, 10500 mg, 10600 mg, 10700 mg, 10800 mg, 10900 mg or 11000 mg of the anti-C5 antibody or antigen binding fragment thereof is administered to a patient weighing ≥60 to <100 kg.

In another embodiment, 10 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, 6100 mg, 6200 mg, 6300 mg, 6400 mg, 6500 mg, 6600 mg, 6700 mg, 6800 mg, 6900 mg, 7000 mg, 7100 mg, 7200 mg, 7300 mg, 7400 mg, 7500 mg, 7600 mg, 7700 mg, 7800 mg, 7900 mg, 8000 mg, 8100 mg, 8200 mg, 8300 mg, 8400 mg, 8500 mg, 8600 mg, 8700 mg, 8800 mg, 8900 mg, 9000 mg, 9100 mg, 9200 mg, 9300 mg, 9400 mg, 9500 mg, 9600 mg, 9700 mg, 9800 mg, 9900 mg, 10000 mg, 10100 mg, 10200 mg, 10300 mg, 10400 mg, 10500 mg, 10600 mg, 10700 mg, 10800 mg, 10900 mg or 11000 mg is administered to a patient weighing ≥100 kg. In some embodiments, dosage regimens are adjusted to provide the optimum desired response (e.g., an effective response).

In another embodiment, the anti-C5 antibody is administered at a milligram per kilogram (mg/kg) dose. In one embodiment, the anti-C5 antibody or antigen binding fragment thereof is administered at a dose of 0.1 mg/kg, 0.25 mg/kg, 0.5 mg/kg, 0.75 mg/kg, 1.0 mg/kg, 1.25 mg/kg, 1.50 mg/kg, 1.75 mg/kg, 2.0 mg/kg, 2.25 mg/kg, 2.50 mg/kg, 2.75 mg/kg, 3.0 mg/kg, 3.25 mg/kg, 3.50 mg/kg, 3.75 mg/kg, 4.0 mg/kg, 4.25 mg/kg, 4.50 mg/kg, 4.75 mg/kg, 5.0 mg/kg, 5.25 mg/kg, 5.50 mg/kg, 5.75 mg/kg, 6.0 mg/kg, 6.25 mg/kg, 6.50 mg/kg, 6.75 mg/kg, 7.0 mg/kg, 7.25 mg/kg, 7.50 mg/kg, 7.75 mg/kg, 8.0 mg/kg, 8.25 mg/kg, 8.50 mg/kg, 8.75 mg/kg, 9.0 mg/kg, 9.25 mg/kg, 9.50 mg/kg, 9.75 mg/kg, 10.0 mg/kg, 11.25 mg/kg, 11.50 mg/kg, 11.75 mg/kg, 12.0 mg/kg, 12.25 mg/kg, 12.50 mg/kg, 12.75 mg/kg, 13.0 mg/kg, 13.25 mg/kg, 13.50 mg/kg, 13.75 mg/kg, 14.0 mg/kg, 14.25 mg/kg, 14.50 mg/kg, 14.75 mg/kg, 15.0 mg/kg, 15.25 mg/kg, 15.50 mg/kg, 15.75 mg/kg, 16.0 mg/kg, 16.25 mg/kg, 16.50 mg/kg, 16.75 mg/kg, 17.0 mg/kg, 17.25 mg/kg, 17.50 mg/kg, 17.75 mg/kg, 18.0 mg/kg, 18.25 mg/kg, 18.50 mg/kg, 18.75 mg/kg, 19.0 mg/kg, 19.25 mg/kg, 19.50 mg/kg, 19.75 mg/kg, 20.0 mg/kg, 20.25 mg/kg, 20.50 mg/kg, 20.75 mg/kg, 21.0 mg/kg, 21.25 mg/kg, 21.50 mg/kg, 21.75 mg/kg, 22.0 mg/kg, 22.25 mg/kg, 22.50 mg/kg, 22.75 mg/kg, 23.0 mg/kg, 23.25 mg/kg, 23.50 mg/kg, 23.75 mg/kg, 24.0 mg/kg, 24.25 mg/kg, 24.50 mg/kg, 24.75 mg/kg or 25.0 mg/kg.

In one embodiment, the anti-C5 antibody is administered once per week, twice per week, three times per week, four times per week, five times per week, six times per week, or daily. In another embodiment, the anti-C5 antibody is administered twice daily. In another embodiment, the anti-C5 antibody is administered once every two weeks, once every three weeks, once every four weeks, once every five weeks, once every six weeks, once every seven weeks, once every eight weeks, once every nine weeks, once every ten weeks, once every eleven weeks, or once every twelve weeks. In another embodiment, the anti-C5 antibody is administered at a loading dose on Day 1, followed by a different maintenance dose on Day 15 and every eight weeks thereafter.

In another embodiment, to obtain an effective response, the anti-C5 antibody is administered to the patient in an amount and with a frequency to maintain a minimum free C5 concentration. In one embodiment, the anti-C5 antibody is administered to the patient in an amount and with a frequency to maintain a free C5 concentration of 0.2 μg/mL, 0.3 μg/mL, 0.4 μg/mL, 0.5 μg/mL or less. In another embodiment, the anti-C5 antibody is administered to the patient in an amount and with a frequency to maintain a free C5 concentration of 0.309 to 0.5 μg/mL or less.

In some embodiments, the patients treated according to the methods described herein have been vaccinated against meningococcal infections within three years prior to, or at the time of, initiating study drug. In one embodiment, patients who initiate treatment less than two weeks after receiving a meningococcal vaccine receive treatment with appropriate prophylactic antibiotics until two weeks after vaccination. In another embodiment, patients treated according to the methods described herein are vaccinated against meningococcal serotypes A, C, Y, W135, and/or B.

### Outcomes

In some embodiments, treatment of MG includes the amelioration or improvement of one or more symptoms associated with MG. Symptoms associated with MG include muscle weakness and fatigability. Muscles primarily affected by MG include muscles that control eye and eyelid movement, facial expressions, chewing, talking, swallowing, breathing, neck movements, and limb movements.

In some embodiments, treatment of MG includes the improvement of a clinical marker for MG progression. These markers include MG-ADL scores, QMG score for disease severity, MGC, NIF, forced vital capacity, MGFA post-intervention status, and other quality of life measurements. In some embodiments, MG-ADL is the primary score for measuring improvement of MG.

The MG-ADL is an 8-point questionnaire that focuses on relevant symptoms and functional performance of activities of daily living (ADL) in MG subjects (Table 3). The 8 items of the MG-ADL were derived from symptom-based components of the original 13-item QMG to assess disability secondary to ocular (2 items), bulbar (3 items), respiratory (1 item), and gross motor or limb (2 items) impairment related to effects from MG. In this functional status instrument, each response is graded 0 (normal) to 3 (most severe). The range of total MG-ADL score is 0-24. A clinically meaningful improvement in a patient's MG-ADL in one embodiment is, for example, a 3 point or greater reduction in score after 26 weeks of treatment.

The current QMG scoring system consists of 13 items: ocular (2 items), facial (1 item), bulbar (2 items), gross motor (6 items), axial (1 item), and respiratory (1 item); each graded 0 to 3, with 3 being the most severe (Table 4). The range of total QMG score is 0-39. The QMG scoring system is an objective evaluation of therapy for MG and is based on quantitative testing of sentinel muscle groups. The MGFA task force has recommended that the QMG score be used in prospective studies of therapy for MG (Benatar, M. et al., Muscle Nerve, 45:909-17, 2012). A clinically meaningful improvement in a patient's QMG in one embodiment is, for example, a 5 point or greater reduction in score after 26 weeks of treatment.

TABLE 3
MG-ADL profile

| Items | Grade 0 | Grade 1 | Grade 2 | Grade 3 | Score (0, 1, 2, 3) |
|---|---|---|---|---|---|
| 1. Talking | Normal | Intermittent | Constant | Difficult to | |
| | | slurring or | slurring or | understand | |
| | | nasal speech | nasal, but can | speech | |
| | | | be understood | | |
| 2. Chewing | Normal | Fatigue with | Fatigue with | Gastric Tube | |
| | | solid food | soft food | | |
| 3. Swallowing | Normal | Rare episode of | Frequent | Gastric Tube | |
| | | choking | choking | | |
| | | | necessitating | | |
| | | | changes in diet | | |
| 4. Breathing | Normal | Shortness of | Shortness of | Ventilator | |
| | | breath with | breath at rest | dependence | |
| | | exertion | | | |
| 5. Impairment of | None | Extra effort, | Rest periods | Cannot do one | |
| ability to brush | | but no rest | needed | of these | |
| teeth or comb hair | | periods needed | | functions | |
| 6. Impairment of | None | Mild, | Moderate, | Severe, | |
| ability to arise from | | sometimes uses | always uses | requires | |
| a chair | | arms | arms | assistance | |
| 7. Double vision | None | Occurs, but not | Daily, but not | Constant | |
| | | daily | constant | | |
| 8. Eyelid drop | None | Occurs, but not | Daily, but not | Constant | |
| | | daily | constant | | |

The MGC is a validated assessment tool for measuring clinical status of subjects with MG (16). The MGC assesses 10 important functional areas most frequently affected by MG and the scales are weighted or clinical significance that incorporates subject-reported outcomes (Table 5; Burns, T. et al., Muscle Nerve, 54:1015-22, 2016). MGC is administered at Screening, Day 1, Weeks 1-4, 8, 12, 16, 20, and 26 or ET (Visits 1-6, 8, 10, 12, 14, and 17 or ET). A clinically meaningful improvement in a patient's MGC in one embodiment is, for example, a 3 point or greater reduction in score after 26 weeks of treatment.

TABLE 5
MG composite scale

| | | | | | | | | |
|---|---|---|---|---|---|---|---|---|
| Ptosis, upward gaze (PE) | >45 | 0 | 11-45 seconds | 1 | 1-10 seconds | 2 | Immediate | 3 |
| | seconds | | | | | | | |
| Double vision on lateral | >45 | 0 | 11-45 seconds | 1 | 1-10 seconds | 2 | Immediate | 4 |
| gaze, left or right (PE) | seconds | | | | | | | |
| Eye closure (PE) | Normal | 0 | Mild weakness (can be | 0 | Moderate weakness | 1 | Severe weakness | 2 |
| | | | forced open with effort) | | (can be forced open | | (unable to keep | |
| | | | | | easily) | | eyes closed) | |
| Talking (Pt) | Normal | 0 | Intermittent slurring or | 2 | Constant slurring or | 4 | Difficult to | 6 |
| | | | nasal speech | | nasal but can be | | understand | |
| | | | | | understood | | | |
| Chewing (Pt) | Normal | 0 | Fatigue with solid food | 2 | Fatigue with soft food | 4 | Gastric tube | 6 |
| Swallowing (Pt) | Normal | 0 | Rare trouble or choking | 2 | Frequent trouble | 5 | Gastric tube | 6 |
| | | | | | (change in diet) | | | |
| Breathing | Normal | 0 | SOB with exertion | 2 | SOB at rest | 4 | Ventilator | 9 |
| Neck Flex/Ext | Normal | 0 | Mild | 1 | Moderate (~50% | 3 | Severe | 4 |
| (weakest PE) | | | | | weak +/− 15%) | | | |
| Shoulder Abd (PE) | Normal | 0 | Mild | 2 | Moderate (~50% | 4 | Severe | 5 |
| | | | | | weak +/− 15%) | | | |
| Hip flexion | Normal | 0 | Mild | 2 | Moderate (~50% | 4 | Severe | 5 |
| | | | | | weak +/− 15%) | | | |
| | | 0 | | 15 | | 33 | | 50 |

The revised Myasthenia Gravis Qualify of Life 15-item scale (MG-QOL15r) is a health-related QoL evaluative instrument specific to patients with MG (Table 6). The MG-QOL15r was designed to provide information about patients' perception of impairment and disability, determine the degree to which disease manifestations are tolerated, and to be administered and interpreted easily. The MG-QOL15r is completed by the patient. Higher scores indicate greater extent of and dissatisfaction with MG-related dysfunction. A clinically meaningful improvement in a patient's MG-QOL15 is a decrease in score after 26 weeks of treatment.

The Neuro-QOL Fatigue is a reliable and validated brief 19-item survey of fatigue completed by the subject or patient. Higher scores indicate greater fatigue and greater impact of MG on activities (Table 7; Gershon, R. et al., Qual. Life Res., 21:475-86, 2012). A clinically meaningful improvement in a patient's Neuro-QQL Fatigue score is reflected in a decrease in score after 26 weeks of treatment.

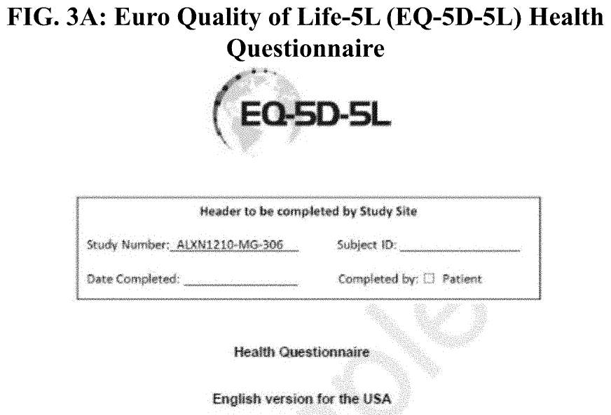

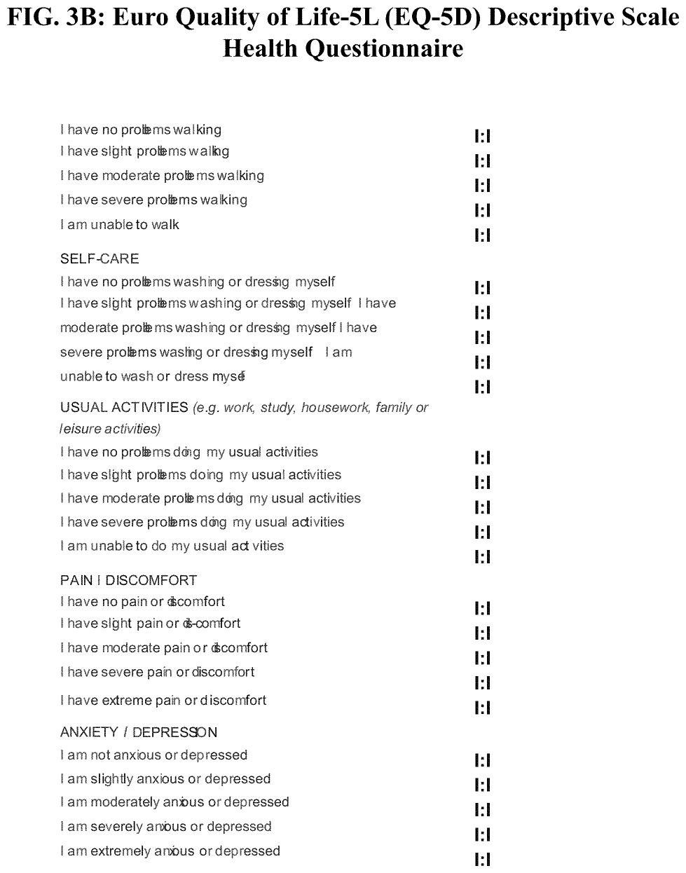

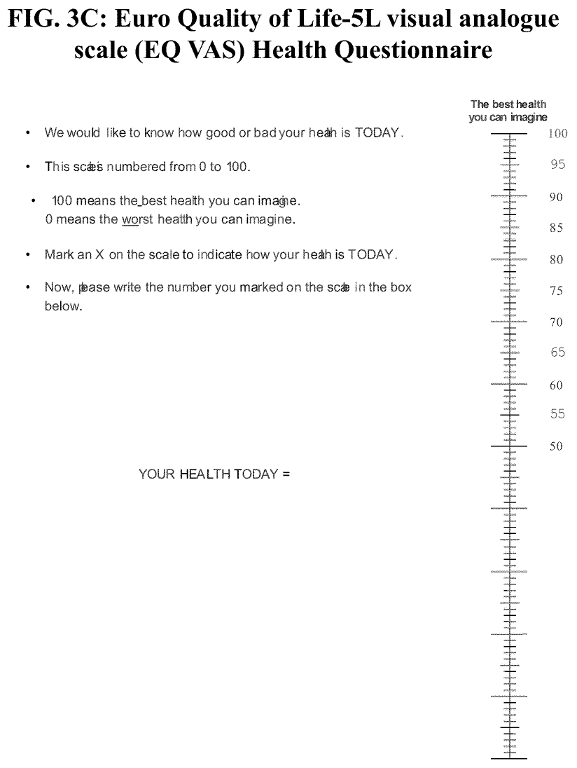

The Euro Quality of Life-5L (EQ-5D-5L) is a self-assessed, health-related QoL questionnaire (FIGS. 3A, 3B and 3C). The EQ-5D-5L essentially consists of 2 pages: the EQ-5D descriptive scale (FIG. 3B) system and the EQ visual analogue scale (EQ VAS) (FIG. 3C). The scale measures QoL on a 5-component scale including mobility, self-care, usual activities, pain/discomfort, and anxiety/depression. Each level is rated on a scale that describes the degree of problems in that area (e.g., I have no problems walking about, slight problems, moderate problems, severe problems, or unable to walk). The patient is asked to indicate his/her health state by ticking the box next to the most appropriate statement in each of the five dimensions. This decision results in a 1-digit number that expresses the level selected for that dimension. The digits for the five dimensions can be combined into a 5-digit number that describes the patient's health state. A clinically meaningful improvement in a patient's EQ 5D is reflected as a decrease in scores in each category after 26 weeks of treatment. This tool also has an overall health scale (EQ VAS) where the rater selects a number between 1-100 to describe the condition of their health, 100 being the best imaginable. The EQ VAS records the patient's self-rated health on a vertical visual analogue scale, where the endpoints are labeled ‘The best health you can imagine’ and ‘The worst health you can imagine.’ The VAS can be used as a quantitative measure of health outcome that reflect the patient's own judgement. A clinically meaningful improvement in a patient's EQ VAS is reflected as an increase in score after 26 weeks of treatment. Convergent validity was demonstrated by a correlation between EQ-5D-5L and the dimensions of World Health Organization 5 Well Being questionnaires, (r=0.43, p<0.001) (see, Janssen, M. et al., Qual. Life Res., 22:1717-27, 2013). The EQ-5D-5L approach is reliable, average test-retest reliability using interclass coefficients with mean of 0.78 and 0.73 (Brooks, R., Health Policy, 37:53-72, 1996; Chaudhury, C. et al., Biochemistry, 45:4983-90, 2006).

Subjects with increasingly severe MG can suffer from potentially fatal respiratory complications including profound respiratory muscle weakness. Respiratory function is monitored closely for evidence of respiratory failure in MG subjects and ventilator support is recommended in the event of consistent declines in serial measurements of Forced Vital Capacity (FVC) or NIF, loss of upper airway integrity (difficulty handling oral secretions, swallowing, or speaking) or in the setting of emerging respiratory failure. FVC as one of the test items in QMG is performed when QMG is performed. NIF was performed using the NIF Meter.

The MG clinical state is assessed using the MGFA Post-Intervention Status (MGFA-PIS). Change in status categories of Improved, Unchanged, Worse, Exacerbation and Died of MG a swell as the Minimal Manifestation (MM) can be assessed (Table 8).

TABLE 8
MGFA-PIS

| | |
|---|---|
| Complete Stable | The patient has had no symptoms or signs of MG for at least 1 year and has received no therapy |
| Remission (CSR) | for MG during that time. There is no weakness of any muscle on careful examination by |
| | someone skilled in the evluation of neuromuscular disease. Isolated weakness of eyelid closure |
| | is accepted. |
| Pharmacologic | The same criteria as for CSR except that the patient continues to take some form of therapy for |
| Remission (PR) | MG. Patients taking cholinesterase inhibitors are excluded from this category because their use |
| | suggests the presence of weakness. |
| Minimal | The patient has no symptoms of functional limitations from MG but has some weakness on |
| Manifestations | examination of some muscles. This class recognizes that some patients who otherwise meet the |
| (MM) | definition of CSR or PR do have weakness that is only detectable by careful examination. |
| MM-0 | The patient has received no MG treatment for at least 1 year. |
| MM-1 | The patient continues to receive some form of immunosuppression but no cholinesterase |
| | inhibitors or other symptomatic therapy. |
| MM-2 | The patient has received only low-dose cholinesterase inhibitors (<120 mg pyridostigmine/day) |
| | for at least 1 year. |
| MM-3 | The patient has received cholinesterase inhibitors or other symptomatic therapy and some form |
| | of immunosuppression during the past year. |
| Change in Status | |
| Improved (I) | A substantial decrease in pretreatment clinical manifestations or a sustained substantial |
| | reduction in MG medications as defined in the protocol. In prospective studies, this should be |
| | defined as specific decrease in QMG score. |
| Unchanged (U) | No substantial change in pretreatment clinical manifestations or reduction in MG medications |
| | as defined in the protocol. In prospective studies, this should be defined in terms of a maximum |
| | change in QMG score. |
| Worse (W) | A substantial increase in pretreatment clinical manifestations or a substantial increase in MG |
| | medications as defined in the protocol. In prospective studies, this should be defined as a |
| | specific increase in QMG score. |
| Exacerbation (E) | Patients who have fulfilled criteria of CSR, PR, or MM but subsequently developed clinical |
| | findings greater than permitted by these criteria. |
| Died of MG (D of | Patients who died of MG, or complications of MG therapy, or within 30 days after thymectomy. |
| MG) | List the cause (see Morbidity and Mortality table). |

Patients administered ravulizumab show a reduced MG-ADL. In some embodiments, the subjects have an initial MG-ADL score of greater than 6 points. In some embodiments, the subjects have an initial MG-ADL score greater than 0, 1, 2, 3, 4, 5, 6, 7, 8, 9, 10, 11, 12, 13, 14, 15, 16, 17, 18, 19, 20, 21, 22 or 23 points. In some embodiments, after a course of treatment with ravulizumab, the MG-ADL score of the subject is reduced to less than 6 points. In some embodiments, the MG-ADL score is reduced at least 1 point, at least 2 points, at least 3 points, at least 4 points, at least 5 points, at least 6 points, at least 7 points, at least 8 points, at least 9 points, at least 10 points, at least 11 points, at least 12 points, at least 13 points, at least 14 points, at least 15 points, at least 16 points, at least 17 points, at least 18 points, at least 19 points, at least 20 points, at least 21 points, at least 22 points, at least 23 points, or at least 24 points after treatment with ravulizumab. In some embodiments, the MG-ADL score of the patient is reduced by at least 1 point after a course of treatment with ravulizumab. In some embodiments, the MG-ADL of the patient is reduced by 1, 2, 3, 4, 5, 6, 7, 8, 9, 10, 11, 12, 13, 14, 15, 16, 17, 18, 19, 20, 21, 22, 23, or 24 points after a course of treatment with ravulizumab.

According to some embodiments, the course of treatment with ravulizumab lasts for 26 weeks. According to some embodiments, the course of treatment lasts for 26-52, 26-78, 26-104, 26-130, 26-156, 26-182, 26-208 weeks, or more. In some embodiments, the course of treatment lasts for greater than 26, 27, 28, 29, 30, 31, 32, 33, 34, 35, 36, 37, 38, 39, 40, 41, 42, 43, 44, 45, 46, 47, 48, 49, 50, 51, 52, 78, 104, 130, 156 or 182 weeks. According to some embodiments, the course of treatment lasts for greater than 1, 2, 3, 4, 5, 10, 15, 20, 25, 30, 35, 40, 45, 50, 55, 60, 65, 70, 75, 80, or more years. In some embodiments, the course of treatment lasts for the remainder of the subject's life.

According to some embodiments, during the course of treatment, one or more symptoms or scores associated with MG improves during the course of treatment and is maintained at the improved level throughout treatment. MG-ADL can improve, for example, after 26 weeks of treatment with a therapeutic antibody that specifically binds C5 and then remain at the improved level for the duration of the treatment, which is 52 weeks of treatment with a therapeutic antibody that specifically binds C5. One example of a therapeutic antibody that binds C5 is ravulizumab.

In some embodiments, the first sign of improvement occurs by 26 weeks of treatment with a therapeutic antibody that specifically binds C5. According to some embodiments, the first sign of improvement occurs between weeks 1-26, 26-52, 52-78, 78-104, 104-130, 130-156, 156-182, or 182-208 of treatment with a therapeutic antibody that specifically binds C5. In some embodiments, the first sign of improvement occurs at week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 78, 104, 130, 156 or 182.

In some embodiments, MG includes refractory gMG. In some embodiments, refractory gMG is characterized as including subjects or patients positive for auto-antibodies binding to AChR who continue to show marked generalized weakness or bulbar signs and symptoms of MG while receiving current standard of care for myasthenia gravis such as cholinesterase inhibitor therapy and IST or who require chronic plasma exchange or chronic IVIg to maintain clinical stability. In some embodiments, refractory gMG is characterized as including subjects or patients who continue to show marked generalized weakness or bulbar signs and symptoms of myasthenia gravis while receiving current standard of care for MG such as cholinesterase inhibitor therapy and IST or who require chronic plasma exchange or chronic IVIg to maintain clinical stability.

### Kits and Unit Dosage Forms

Also provided herein are kits that include a pharmaceutical composition containing an anti-C5 antibody or antigen binding fragment thereof, such as ravulizumab, and a pharmaceutically acceptable carrier, in a therapeutically effective amount adapted for use in the preceding methods. The kits can also optionally include instructions, e.g., comprising administration schedules, to allow a practitioner (e.g., a physician, nurse or patient) to administer the composition contained therein to administer the composition to a patient having MG. The kit also can include a syringe.

Kits can optionally include multiple packages of the single-dose pharmaceutical compositions each containing an effective amount of the anti-C5 antibody or antigen binding fragment thereof for a single administration in accordance with the methods provided above. Instruments or devices necessary for administering the pharmaceutical composition(s) also may be included in the kits. A kit may provide one or more pre-filled syringes containing an amount of the anti-C5 antibody or antigen binding fragment thereof.

The following examples are merely illustrative and should not be construed as limiting the scope of this disclosure in any way as many variations and equivalents will become apparent to those skilled in the art upon reading the present disclosure. The contents of all references, Genbank entries, patents and published patent applications cited throughout this application are expressly incorporated herein by reference.

### EXAMPLES

### Example 1: A Phase 3, Randomized, Double-Blind, Placebo-Controlled, Multicenter Study to Evaluate the Safety and Efficacy of Ravulizumab in Complement-Inhibitor-Naïve Adult Patients with Generalized Myasthenia Gravis

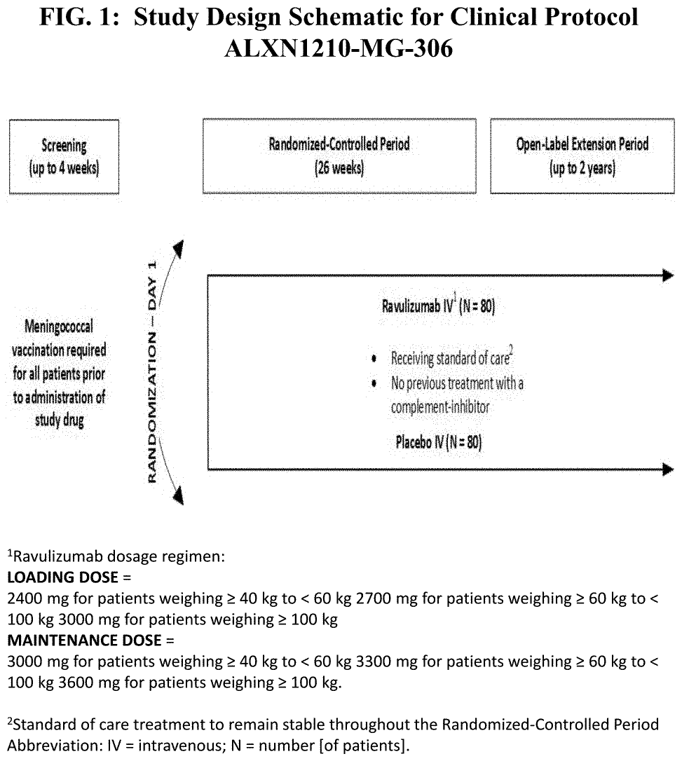

A Phase 3, randomized, double-blind, placebo-controlled, multicenter study is conducted to evaluate the safety and efficacy of ravulizumab administered by intravenous (IV) infusion to adult patients with gMG. The ALXN1210-MG-306 study schematic is shown in FIG. 1.

1. Study Rationale

Ravulizumab specifically binds the human terminal complement component (C5) with high affinity, inhibiting C5 enzymatic cleavage and thereby preventing the generation of the proinflammatory/prothrombotic complement activation products, C5a, and the cytolytic and proinflammatory/prothrombotic membrane attack complex, C5b-9, which are responsible for the antibody-mediated destruction of the NMJ, loss of acetylcholine receptors, and failure of neuromuscular transmission associated with gMG. Eculizumab is approved for the treatment of, for example, gMG, under the trade name Soliris®.

Like eculizumab, ravulizumab also provides essentially immediate and complete C5 inhibition, but ravulizumab further provides sustained complement inhibition throughout a prolonged dosing interval; it was specifically designed (and has subsequently been proven) to have an increased half-life relative to eculizumab. Ravulizumab therefore requires less frequent (once every 8 weeks [q8w]) infusions than eculizumab (once every 2 weeks [q2w] infusions). Given that gMG is a chronic disease with a significant treatment burden, the relative convenience of the ravulizumab dosing regimen may increase patient satisfaction and treatment-adherence, and ultimately, lead to improved health-outcomes.

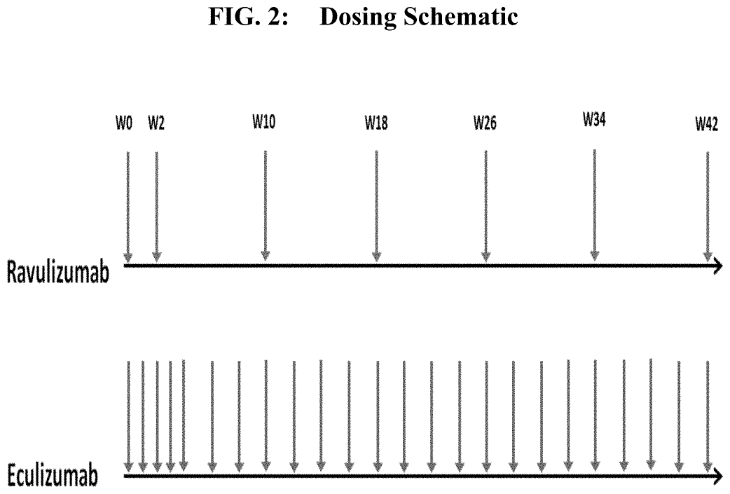

The enhanced pharmacokinetic (PK)/pharmacodynamic profile of ravulizumab, with fewer PK troughs than eculizumab, has the potential to improve therapeutic efficacy while maintaining a safety profile similar to that of eculizumab. The q8w dosing regimen minimizes the risk of incomplete complement inhibition. The infusion frequency is relatively low (6 infusions per year) (FIG. 2), which offers the potential for improved quality of life (QoL) through fewer missed days of work or school, better treatment adherence, and improved accessibility. Ravulizumab offers a convenient dosing and immediate onset of action with effective and complete terminal complement inhibition at the end of the first infusion. The dose regimen of ravulizumab has been optimized to reduce the exposure differences across the adult body-weight range by utilizing a weight-based dosing paradigm that provides immediate, complete, and sustained C5 inhibition over the entire dosing interval. Therefore, ravulizumab minimizes the risk of inflammation, including C5a recruitment and activation of inflammatory cells as well as direct MAC-complex induced damage of the motor neural endplate (Kusner, L. et al., Expert Rev. Clin. Immunol., 4:43-52, 2008).

2. Risk Benefit Assessment

Ravulizumab provides patients and physicians with an option for less frequent dosing, which allows greater access to care for those patients who may not initiate treatment on eculizumab, may discontinue eculizumab due to frequency of dosing, or who are currently receiving eculizumab every 2 weeks.

Neisseria meningitidis

Increased susceptibility to infection caused by Neisseria meningitidis (N. meningitidis) is a known risk associated with complement inhibition. The main risk associated with ravulizumab is the risk of meningococcal infections. Specific risk mitigation measures are in place to address this risk, as described herein.

Immunogenicity

Administration of any therapeutic protein, including ravulizumab, may induce an immunogenic response potentially resulting in antidrug antibodies (ADA). The spectrum of potential clinical consequences may include severe hypersensitivity-type reactions and decrease in efficacy (PK and/or PD neutralization) due to development of neutralizing ADA (Casadevall, N. et at., N. Engl. J. Med., 346:469-75, 2002; Li, J. et al., Blood, 98:3241-8, 2001).

Of the 261 patients with paroxysmal nocturnal hemoglobinuria (PNH) who were treated with ravulizumab in the ravulizumab IV clinical studies, 1 patient developed a treatment-emergent ADA. Treatment-emergent ADAs have been observed in 3 healthy subjects treated with ravulizumab subcutaneous (SC) and 1 healthy subject treated with ravulizumab IV in Study ALXN1210-HV-104. All ADA positive titer values were low and negative for eculizumab cross-reactivity. There was no apparent impact of immunogenicity on the PK or PD of ravulizumab.

Monitoring of immunogenicity for this study is conducted as described in Table 10 and Table 11 and as described otherwise herein.

Local and Systemic Reactions

Protein therapies administered IV have the potential risk of causing local (infusion-site reactions) and systemic reactions (infusion-associated reactions). Infusion-site reactions are those localized to the site of IV drug administration and may include reactions such as erythema, pruritus and bruising. Infusion-associated reactions are those that are systemic in nature and that may be immune or nonimmune-mediated, generally occurring within hours of drug administration. Immune-mediated reactions may include allergic reactions (e.g., anaphylaxis), while nonimmune-mediated reactions are nonspecific (e.g., headache, dizziness, nausea). Monitoring for these reactions is conducted as part of routine safety assessments for this study as described herein.

3. Objectives

The primary objective of the study is to assess the efficacy of ravulizumab compared with placebo in the treatment of gMG based on the improvement in the MG-ADL profile. The secondary objective of the study is to assess the efficacy of ravulizumab compared with placebo in the treatment of gMG based on the improvement in the QMG total score.

Exploratory objectives of this study are to (1) evaluate the PK/PD and immunogenicity of ravulizumab in the treatment of gMG throughout the study, (2) assess the efficacy of ravulizumab compared to placebo in the treatment of gMG based on the incidence of all-cause hospitalization or Clinical Deterioration, (3) assess the efficacy of ravulizumab compared with placebo in the treatment of gMG based on the improvement in quality of life measures, and (4) assess the efficacy of ravulizumab in the treatment of gMG based on other efficacy endpoints throughout the study.

The safety objective of this study is to characterize the overall safety of ravulizumab in the treatment of gMG.

4. Endpoints

The primary efficacy endpoint of the study is change from baseline in MG-ADL total score at Week 26 of the Randomized-Controlled Period.

The secondary efficacy endpoint of the study is Change from Baseline in QMG total score at Week 26.

The exploratory efficacy endpoints of the study include the following:
Change in serum ravulizumab concentration over time.
Change in free serum C5 concentration over time;
Incidence of treatment-emergent antidrug antibodies overtime;
Incidence of all-cause hospitalization or Clinical Deterioration during the 26 weeks of the Randomized-Controlled Period;
Change from Baseline in the Revised 15-Component Myasthenia Gravis Quality of Life (MG-QOL15r) score at Week 26;
Change from Baseline in Neuro-QOL Fatigue score at Week 26;
Improvement of at least 3 points in the MG-ADL total score from Baseline at Week 26;
Improvement of at least 5 points in the QMG total score from Baseline at Week 26;
Change from Baseline in the Myasthenia Gravis Composite (MGC) score at Week 26;
Myasthenia Gravis Foundation of America (MGFA) Post-Intervention Status (PIS) at Week 26;
Change from Baseline in Euro Quality of Life (EQ-5D-5L) at Week 26.

The safety endpoints of this study are (1) incidence of adverse events and serious adverse events over time and (2) changes from Baseline in vital signs and laboratory assessments.

The objectives and endpoints of the study are summarized in Table 9 herein.

TABLE 9
Study ALXN1210-MG-306 objectives and endpoints

| | |
|---|---|
| Objectives | Endpoints |

Primary

| | |
|---|---|
| To assess the efficacy of ravulizumab compared with | Change from Baseline in MG-ADL total score at |
| placebo in the treatment of gMG based on the | Week 26 of the Randomized-Controlled Period. |
| improvement in the Myasthenia Gravis-Activities of | |
| Daily Living (MG-ADL) profile. | |

Secondary

| | |
|---|---|
| To assess the efficacy of ravulizumab compared with | Change from Baseline in QMG total score at Week 26. |
| placebo in the treatment of gMG based on the | |
| improvement in the Quantitative Myasthenia Gravis | |
| (QMG) total score. | |

Exploratory

| | |
|---|---|
| To evaluate the PK/PD and immunogenicity | Change in serum ravulizumab concentration |
| of ravulizumab in the treatment of gMG | over time. |
| throughout the study. | Change in free serum C5 concentration over |
| | time. |
| | Incidence of treatment-emergent antidrug |
| | antibodies over time. |
| To assess the efficacy of ravulizumab | Incidence of all-cause hospitalization or |
| compared to placebo in the treatment of | Clinical Deterioration during the 26 weeks of |
| gMG based on the incidence of all-cause | the Randomized-Controlled Period. |
| hospitalization or Clinical Deterioration. | |
| To assess the efficacy of ravulizumab | Change from Baseline in the Revised 15- |
| compared with placebo in the treatment of | Component Myasthenia Gravis Quality of |
| gMG based on the improvement in quality | Life (MG-QOL15r) score at Week 26. |
| of life measures. | Change from Baseline in Neuro-QOL |
| | Fatigue score at Week 26. |
| To assess the efficacy of ravulizumab in the | Improvement of at least 3 points in the MG- |
| treatment of gMG based on other efficacy | ADL total score from Baseline at Week 26. |
| endpoints throughout the study. | Improvement of at least 5 points in the QMG |
| | total score from Baseline at Week 26. |
| | Change from Baseline in the Myasthenia |
| | Gravis Composite (MGC) score at Week 26. |
| | Myasthenia Gravis Foundation of America |
| | (MGFA) Post-Intervention Status (PIS) at |
| | Week 26. |
| | Change from Baseline in Euro Quality of |
| | Life (EQ-5D-5L) at Week 26. |

Safety

| | |
|---|---|
| To characterize the overall safety of ravulizumab in the | Incidence of adverse events and serious |
| treatment of gMG. | adverse events over time. |
| | Changes from Baseline in vital signs and |
| | laboratory assessments. |

5. Overall Design

ALXN1210-MG-306 is a Phase 3, randomized, double-blind, parallel-group, placebo-controlled, multicenter study to evaluate the safety and efficacy of ravulizumab for the treatment of patients with gMG. The ALXN1210-MG-306 study schematic is shown in FIG. 1. Approximately 160 eligible patients are stratified by region (North America, Europe, Asia Pacific, and Japan) and randomized 1:1 to 1 of 2 treatment groups: (1) ravulizumab infusion or (2) placebo infusion. There are 3 periods in this study: Screening Period, Randomized-Controlled Period, and an Open-Label Extension (OLE) Period.

After the 26-Week Randomized-Controlled Period and assessments on Day 183 (Week 26), patients in the placebo group receive a blinded loading dose of ravulizumab and patients in the ravulizumab group receive a blinded ravulizumab dose of 900 mg. Starting Week 28, all patients begin open-label ravulizumab maintenance doses q8w. For patients in the ravulizumab group, a blinded ravulizumab dose of 900 mg is chosen to ensure maintenance of complete C5 inhibition until the next scheduled maintenance dose at Week 28 (Day 197).

Eight weeks after the final dose of study drug is administered, all enrolled patients return for an End of Study (EOS) Visit (Visit 30) at Week 132 (2 days) during which final study assessments are conducted. If a patient withdraws from the study, or completes the study early (prior to Visit 29; Week 124), for example if ravulizumab has become registered or approved (in accordance with country-specific regulations) prior to Visit 29, the patient is encouraged to return for an Early Termination (ET)/EOS Visit, 8 weeks (±2 days) after the day the last dose of study drug is administered, during which final planned safety assessments are conducted as described herein. Attempts are made to follow all patients for safety for 8 weeks from the day the last dose of study drug is administered.

Patients who are being treated with an IST at the time of the Screening Visit may continue taking their baseline ISTs throughout the Randomized-Controlled and OLE Periods. The dosage of IST, however, must not be changed and no new ISTs may be added or discontinued during the Randomized-Controlled Period of the study, unless deemed by the Investigator to be medically necessary. Throughout the study, rescue therapy (e.g., high-dose corticosteroids, plasmapheresis/plasma exchange, or intravenous immunoglobulin) are allowed if a patient experiences Clinical Deterioration, as defined by the study protocol herein. The rescue therapy used for a particular patient is at the discretion of the Investigator.

Throughout the study, rescue therapy (e.g., high-dose corticosteroid, PP/PE, or IVIg) are allowed if a patient experiences Clinical Deterioration as defined herein. The rescue therapy used for a particular patient is at the discretion of the Investigator.

The primary endpoint for this study is measured at Week 26 (Day 183). Endpoints are measured and analyzed irrespective of rescue therapy. For those patients who complete the study, as defined in the protocol, the EOS Visit is defined as patient's last visit in the (up to) 2-year OLE Period. Including the 8-week safety follow-up, which begins after the patient's last dose of study drug is administered, the overall study-duration for an individual patient is estimated to take up to 132 weeks (from enrollment through the end of the Safety Follow-up). The period of active patient-participation is estimated to take up to 132 weeks (from enrollment through the EOS Visit).

Schedules of Activities (SOA) for the Randomized-Controlled Period and the OLE Period are provided in Table 10 and Table 11, respectively.

Screening Period (2-4 Weeks Prior to Day 1)

At the screening visit, after obtaining informed consent, the patient is screened for study eligibility through medical history review, demographic data, and laboratory assessments. The medical history review includes confirmation of MG diagnosis as defined in the inclusion criteria of this protocol, history of previous treatment/therapies for MG (e.g., thymectomy, ISTs including corticosteroids, IVIg and PE/PP), history of MG exacerbation or crisis including the duration of each exacerbation/crisis, the medication taken at the time of each exacerbation/crisis, and the treatment for each exacerbation/crisis.

If all inclusion criteria and none of the exclusion criteria are met, patients are vaccinated against N. meningitidis, if not already vaccinated within the 3 years prior to their enrollment in the study. Patients who initiate study drug treatment less than 2 weeks after receiving a meningococcal vaccine receive treatment with appropriate prophylactic antibiotics until 2 weeks after vaccination.

If a patient experiences a Clinical Deterioration or MG Crisis during the Screening Period, the Sponsor is notified. Following discussion with the Sponsor, a decision is made about whether the patient may continue in the study.

Number of Patients

Patients are screened until enough patients have been enrolled to achieve an estimated total of 160 patients, with approximately 80 patients per group.

Randomization

At the time of randomization, all patients are reassessed for eligibility based on the study inclusion and exclusion criteria. All patients who are vaccinated, continue to meet all of the inclusion criteria and none of the exclusion criteria at Randomization [Day 1]), and have been cleared for randomization by the Investigator, are randomized 1:1 to 1 of 2 treatment groups: (1) ravulizumab infusion or (2) placebo infusion. Patients are centrally randomized using interactive response technology. The randomization is stratified by region (North America, Europe, Asia-Pacific, and Japan).

Throughout the study, rescue therapy (e.g., high-dose corticosteroid, PP/PE or IVIg) is allowed when a patient's health would be in jeopardy if rescue therapy is not administered (e.g., emergent situations), or if a patient experiences Clinical Deterioration as defined in this protocol. The rescue therapy used for a particular patient is at the discretion of the Investigator.

Patients are informed of potential signs and symptoms of Clinical Deterioration or MG Crisis and instructed to contact the Investigator to be evaluated within 48 hours of notification of the Investigator of the symptom onset. At the evaluation visit, the Investigator or the Investigator's designee perform the assessments as specified by this protocol. The Investigator or designee determine whether or not the patient meets the definition of Clinical Deterioration as defined herein, and treat the patient accordingly.

The primary endpoint for this study is measured at Week 26 (Day 183), irrespective of rescue therapy.

Patients randomized to the ravulizumab group receive a blinded loading dose of ravulizumab on Day 1, followed by blinded maintenance doses of ravulizumab on Day 15 (Week 2) and q8w thereafter, for a total of 18 weeks of treatment. Patients randomized to placebo receive a blinded dose of placebo on Day 1, followed by blinded doses of placebo on Day 15 (Week 2) and q8w thereafter, for a total of 18 weeks. Both ravulizumab and placebo are administered by intravenous infusion.

After the 26-Week Randomized-Controlled Period and assessments on Day 183 (Week 26), patients in the placebo group receive a blinded loading dose of ravulizumab and patients in the ravulizumab group receive a blinded ravulizumab dose of 900 mg; the 900 mg dose is chosen to ensure maintenance of complete C5 inhibition until the next scheduled maintenance dose at Week 28 (Day 197). Starting at Week 28, all patients begin open-label ravulizumab maintenance doses q8w.

The OLE Period for each patient commences when the patient receives a dose of ravulizumab on Week 26 (Day 183) and continues for up to 2 years or until the product is registered or approved (in accordance with country-specific regulations), whichever occurs first.

The Schedule of Activities for Screening Through End of the Randomized-Controlled Period is shown in Table 10 and through the Extension Period is shown in Table 11.

TABLE 10
Schedule of activities: screening through end of the Randomized-Controlled period

| Period/Phase | Screening | Randomized-Controlled Period | | | | | | | | | | | | Clinical |
|---|---|---|---|---|---|---|---|---|---|---|---|---|---|---|
| Study Visit | 1 | 2 | 3 | 4 | 5 | 6 | 7 | 8 | 9 | 10 | 11 | 12 | 13/ET² | Deterioration [symbol] ¹ |
| Study Day | | D1 | D8 | D15 | D22 | D29 | D57 | D71 | D85 | D99 | D127 | D155 | D183 | |
| Window(day) | | | ±2 | ±2 | ±2 | ±2 | ±2 | ±2 | ±2 | ±2 | ±2 | ±2 | ±2 | |
| Weeks | −4 to −2 W | | W1 | W2 | W3 | W4 | W8 | W10 | W12 | W14 | W18 | W22 | W26 | |
| Informed Consent | X | | | | | | | | | | | | | |
| Assessment of | X | X | | | | | | | | | | | | |
| Inclusion/Exclusion | | | | | | | | | | | | | | |
| Criteria | | | | | | | | | | | | | | |
| Medical History | X | | | | | | | | | | | | | |
| MG History | X | | | | | | | | | | | | | |
| MGFA Clinical | X | X | | | | | | | | | | | | |
| Classification³ | | | | | | | | | | | | | | |
| Weight | X | | X | | | | X | | | X | | X | X | |
| Height | X | | | | | | | | | | | | | |
| HIV-(1 and 2) testing | X | | | | | | | | | | | | | |
| ^{Vital Signs} & Pulse | X | X | X | X | X | X | X | X | X | X | X | X | X | X |
| Oximetry⁴ | | | | | | | | | | | | | | |
| Physical Examination | X | | | | | | | | | | | | X | X |
| Abbreviated physical | | X | X | X | X | X | X | X | X | X | X | X | | |
| Examination⁵ | | | | | | | | | | | | | | |
| Concomitant Medication | X | X | X | X | X | X | X | X | X | X | X | X | X | X |
| Non-Drug Therapy | X | X | X | X | X | X | X | X | X | X | X | X | X | X |
| MG Therapy Status | X | X | X | X | X | X | X | X | X | X | X | X | X | X |
| Hospitalization Status | | X | X | X | X | X | X | X | X | X | X | X | X | X |
| Adverse Event | X | X | X | X | X | X | X | X | X | X | X | X | X | X |
| MG-QOL15r | X | X | X | X | X | X | X | X | X | X | X | X | X | X |
| Neuro-QOL Fatigne | X | X | X | X | X | X | X | X | X | X | X | X | X | X |
| EQ-5D-5L | X | X | X | X | X | X | X | X | X | X | X | X | X | X |
| MG-ADL^{3,6} | X | X | X | X | X | X | X | X | X | X | X | X | X | X |
| _{QMG}3, 7 | X | X | X | X | X | X | X | X | X | X | X | X | X | X |
| _{MGC}3, 7 | X | X | X | X | X | X | X | X | X | X | X | X | X | X |
| MGFA-PIS³ | | X | | | | X | X | | X | X | X | X | X | |
| C-SSRS | | X | | | | | | | | | | | | |
| Baseline/Screening | | | | | | | | | | | | | | |
| Version | | | | | | | | | | | | | | |
| C-SSRS Since Last Visit | | | | | | | | | X | X | | | X | |
| Version | | | | | | | | | | | | | | |
| ECG | X | | | | | | | | | | | | | |
| AChR Ab | X | | | | | | | | X | | | | X | X |
| Clinical Lab Tests⁸ | X | X | | X | | | | X | | | X | | X | X |
| Pregnancy Test⁹ | X | X | | X | | | | X | | | X | | X | |
| PK, Free C5¹⁰ | | B/P | | T/P | | | | T/P | | | T/P | | T | X |
| ADA¹⁰ | | X | | | | | | X | | | X | | X | X |
| N menigitidis Vaccine¹¹ | X | | | | | | | | | | | | | |
| Patient Safety Information | | X | X | X | X | X | X | X | X | X | X | X | X | |
| Card¹² | | | | | | | | | | | | | | |
| Randomization¹³ | | X | | | | | | | | | | | | |
| Study Drug Infusion¹⁴ | | X | | X | | | | X | | | X | | | |

¹Evaluation of Clinical Deterioration is performed as soon as possible, within 48 hours of notification to the Investigator of symptom onset. Additional evaluation visits are scheduled at the discretion of the Investigator.
²If a patient withdraws early from the study during the Randomized-Controlled Period an Early Termination Visit is performed.
³Refer, e.g., to Table 3,
⁴Vital signs and pulse oximetry include systolic and diastolic blood pressure (millimeters of mercury [mmHg]), pulse oximetry (oxygen saturation [SO2]), heart rate (beats/minute), and temperature (degrees Celsius [° C.] or degrees Fahrenheit [° F.]). On dosing days, vital signs are taken before study drug administration and after the patient has been resting for at least 5 minutes.
⁵Are performed, if necessary, on the basis of the patient's health status and the clinical judgement of the Investigator.
⁶The MG-activities of daily living (MG-ADL) assessment is performed by a Properly Trained Clinical Evaluator, preferably the same evaluator, throughout the study. The recall period for MG-ADL is the preceding 7 days or since the last visit if the visit interval is less than 7 days.
7If a patient is taking a cholinesterase inhibitor, the dose is withheld for at least 10 hours prior to the assessment.
⁸Clinical laboratory tests are performed at the central laboratory.
⁹Pregnancy tests ale performed on all patients of child-bearing potential at the specified time points. Serum pregnancy test are performed at Screening; urine pregnancy tests are performed at all other required time points. A negative urine test result is required prior to administering ravulizumab to patients of childbearing potential at the indicated visits. Additional pregnancy tests (urine or serum) may also be performed at any visit at the Investigator's discretion.
¹⁰Baseline (B) and trough (T) blood samples for serum PK, free C5 (PD), and ADA are collected predose (within 30 minutes prior to the start of infusion of study drug). Peak (P) blood samples for serum PK,IPD samples are taken within the 30 minutes following completion of study drug infusion, The T samples are drawn through the venous access created for the dose infusion, prior to administration of the dose. The P samples are drawn from the patient's opposite, noninfused am. On Day 183 (Week 26), the T sample is considered a Randomized-Controlled Period assessment and the P sample is considered an Extension Period assessment. All collection times are recorded in eCRF. In the event of Clinical Deterioration, blood samples for serum PK/PD and ADA analyses are collected if supplemental dosing is described herein.
¹¹To reduce the risk of meningococcal infection (N. meningitidis), all patients are vaccinated against meningococcal infections within 3 years prior to, or at the time of, initiating study drug. Patients who initiate study drug treatment less than 2 weeks after receiving a meningococcal vaccine receive treatment with appropriate prophylactic antibiotics until 2 weeks after vaccination.
¹²Patients are given a Patient Safety Information Card prior to the first dose of study drug. At each visit throughout the study, the study staff ensures that the patient has the Patient Safety Information Card.
¹³All patients that continue to meet all inclusion criteria and none of the exclusion criteria and have been cleared for randomization by the Investigator are centrally randomized through interactive response technology (IRT).
¹⁴Study drug is administered intravenously via infusion after completion of all other tests and procedures, excluding the peak blood sampling for PK/PD, free C5, and ADA,.
Abbreviations:
AChR Ab = acetylcholine receptor antibody;
ADA = antidrug antibody;
B = baseline sample;
C5 = complement component 5;
C-SSRS = Columbia-Suicide Severity Rating Scale;
D = day;
ECG = electrocardiogram;
EQ-5D-5L = Euro Quality of Life;
ET = Early Termination;
HIV = Human Immunodeficiency Virus;
MG = Myasthenia Gravis;
MG-ADL = Myasthenia gravis Activities of Daily Living profile;
MGC = Myasthenia gravis Composite score;
MGFA = Myasthenia Gravis Foundation of America;
MGFA-PIS = MGFA-Post-Intervention Status;
N. meningitidis = Neisseria meningitidis;
P = peak sample;
PK/PD = pharmacokinetic(s)/pharmacodynamic(s);
QMG = Quantitative Myasthenia Gravis score for disease severity;
QoL = quality of life;
T = trough sample;
W = week(s). [symbol]
indicates data missing or illegible when filed

TABLE 11
Schedule of activities: Extension period

| | | | | | | | | | | | | | | | | | | | Clini- |
|---|---|---|---|---|---|---|---|---|---|---|---|---|---|---|---|---|---|---|---|
| | | | | | | | | | | | | | | | | | | | cal |
| Period | | | | | | | | | | | | | | | | | | | Dete- |
| Study | Open-Label Extension | | | | | | | | | | | | | | | | | | rio- |
| Visit | 13 | 14 | 15 | 16 | 17 | 18 | 19 | 20 | 21 | 22 | 23 | 24 | 25 | 26 | 27 | 28 | 29 | 30 | ration¹ |
| Study | D183 | D197 | D211 | D253 | D267 | D281 | D309 | D365 | D421 | D477 | D533 | D533 | D645 | D701 | D757 | D813 | D869 | D925/ | |
| Days² | | | | | | | | | | | | | | | | | | ET³/ | |
| | | | | | | | | | | | | | | | | | | EOS | |
| Weeks | W26 | W28 | W30 | W36 | W38 | W40 | W44 | W52 | W60 | W68 | W76 | W76 | W92 | W100 | W108 | W116 | W124 | W132 | |
| Window | ±2 | ±2 | ±2 | ±2 | ±2 | ±2 | ±2 | ±2 | ±2 | ±2 | ±2 | ±2 | ±2 | ±2 | ±2 | ±2 | ±2 | ±2 | |
| (day) | | | | | | | | | | | | | | | | | | | |
| Weight | | X | X | | | X | X | X | X | X | X | X | X | X | X | X | X | X | |
| Vital | | X | X | X | X | X | X | X | X | X | X | X | X | X | X | X | X | X | X |
| Signs & | | | | | | | | | | | | | | | | | | | |
| Pulse | | | | | | | | | | | | | | | | | | | |
| Oximetry⁴ | | | | | | | | | | | | | | | | | | | |
| Physical | | | | | | | | | | | | | | | | | | X | X |
| Examina- | | | | | | | | | | | | | | | | | | | |
| tion | | | | | | | | | | | | | | | | | | | |
| Abbrevi- | | X | X | X | X | X | X | X | X | X | X | X | X | X | X | X | X | | |
| ated | | | | | | | | | | | | | | | | | | | |
| Physical | | | | | | | | | | | | | | | | | | | |
| Examina- | | | | | | | | | | | | | | | | | | | |
| tion⁵ | | | | | | | | | | | | | | | | | | | |
| Concomi- | | X | X | X | X | X | X | X | X | X | X | X | X | X | X | X | X | X | X |
| tant | | | | | | | | | | | | | | | | | | | |
| Medica- | | | | | | | | | | | | | | | | | | | |
| tion | | | | | | | | | | | | | | | | | | | |
| Non-Drug | | X | X | X | X | X | X | X | X | X | X | X | X | X | X | X | X | X | X |
| Therapy | | | | | | | | | | | | | | | | | | | |
| MG | | X | X | X | X | X | X | X | X | X | X | X | X | X | X | X | X | X | X |
| Therapy | | | | | | | | | | | | | | | | | | | |
| Status | | | | | | | | | | | | | | | | | | | |
| Hospitali- | | X | X | X | X | X | X | X | X | X | X | X | X | X | X | X | X | X | X |
| zation | | | | | | | | | | | | | | | | | | | |
| Status | | | | | | | | | | | | | | | | | | | |
| Adverse | | X | X | X | X | X | X | X | X | X | X | X | X | X | X | A | A | X | X |
| Events | | | | | | | | | | | | | | | | | | | |
| MG-QOL- | | X | X | X | X | X | X | X | X | X | X | X | X | X | X | X | X | X | X |
| 15r | | | | | | | | | | | | | | | | | | | |
| Neuro- | | X | X | X | X | X | X | X | X | X | X | X | X | X | X | X | X | X | X |
| QOL | | | | | | | | | | | | | | | | | | | |
| Fatigue | | | | | | | | | | | | | | | | | | | |
| EQ-5D-5L | | X | X | X | X | X | X | X | X | X | X | X | X | X | X | X | X | X | X |
| MG- | | X | X | X | X | X | X | X | X | X | X | X | X | X | X | X | X | X | X |
| ADL^{6,7} | | | | | | | | | | | | | | | | | | | |
| _{QMG}6, 8 | | X | X | X | X | X | X | X | X | X | X | X | X | X | X | X | X | X | X |
| _{MGC}6, 8 | | X | X | X | X | X | X | X | X | X | X | X | X | X | X | X | X | X | |
| MGFA- | | | | | X | | X | | X | | X | | X | | X | | X | X | |
| PIS⁶ | | | | | | | | | | | | | | | | | | | |
| C-SSRS | | | | | | X | | X | | X | | X | | X | | X | | X | X |
| Since | | | | | | | | | | | | | | | | | | | |
| Last Visit | | | | | | | | | | | | | | | | | | | |
| Version | | | | | | | | | | | | | | | | | | | |
| ECG | | | | | | | | | | | | | | | | | | X | |
| AChR Ab | | | | | | | X | | X | | X | | X | | X | | X | X | X |
| Clinical | | X | | X | | X | X | X | X | X | X | X | X | X | X | X | X | X | X |
| Lab | | | | | | | | | | | | | | | | | | | |
| Tests⁹ | | | | | | | | | | | | | | | | | | | |
| Pregnancy | | X | | X | | X | X | X | X | X | X | X | X | X | X | X | X | X | |
| Test¹⁰ | | | | | | | | | | | | | | | | | | | |
| PK Free | P | T/P | | T/P | | | T/P | T/P | T/P | T/P | T/P | T/P | T/P | T/P | T/P | T/P | T/P | | X |
| C511 | | | | | | | | | | | | | | | | | | | |
| ADA¹¹ | | X | | X | | | X | X | X | X | X | X | X | X | X | X | X | | X |
| Patient | | X | X | X | X | X | X | X | X | X | X | X | X | X | X | X | X | | |
| Safety | | | | | | | | | | | | | | | | | | | |
| informa- | | | | | | | | | | | | | | | | | | | |
| tion | | | | | | | | | | | | | | | | | | | |
| Card¹² | | | | | | | | | | | | | | | | | | | |
| Ravulizu- | X | X | | X | | | X | X | X | X | X | X | X | X | X | X | X | | |
| mab | | | | | | | | | | | | | | | | | | | |
| Infusion¹³ | | | | | | | | | | | | | | | | | | | |

¹Evaluation of or Clinical Deterioration is performed as soon as possible, within 48 hours of notification to the Invesigator of symptom onset. Additional evaluation visits are scheduled at the discretion of the Investigator.
²Extension Period begins at the start of Day 183 (Week 26) dosing.
³If a patient withdraws early from the study during the Extension Period an Early Termination Visit is performed.
⁴Vital signs and pulse oximetry include systolic and diastolic blood pressure (millimeters of mercury [mmHg]), pulse oximetry (oxygen saturation [SO₂]), heart rate (beats/minute), and temperature (degrees Celsius [° C.] or degrees Fahrenheit [° F.]). On dosing days, vital signs are taken before study drug administration and after the patient has been resting for at least 5 minutes.
⁵Are performed, if necessary, on the basis of the patient's health status and the clinical judgement of the Investigator.
⁶Refer, e.g., to Table 3.
⁷The MG-ADL is performed by a Properly Trained Clinical Evaluator, preferably the same evaluator, throughout the study. The recall period for MG-ADL is the preceding 7 days or since the last visit if the visit interval is less than 7 days.
8If a patient is taking a cholinsterase inhibitor, the dose is withheld for at least 10 hours prior to the assessment,
⁹Clinical laboratory tests are performed at the central laboratory.
¹⁰Pregnancy tests are performed on all patients of child-bearing potential at the specified time points. Serum pregnancy tests are performed at Day 925/ET/EOS; urine pregnancy tests are performed at all other required time points. A negative urine test result is required prior to administering ravulizumab to patients of childbearing potential at the indicated visits. Additional pregnancy test (urine or serum) may also be performed at any visit at the Investigator's discretion.
¹¹Trough (T) blood samples for serum PK, free C5 (PD), and ADA are collected predose (within 30 minutes prior to the start of infusion of study drug). Peak (P) blood samples for serum PK/PD are taken within the 30 minutes following completion of study drug infusion. The T samples are drawn through the venous access created for the dose infusion, prior to administration of the dose. The P samples are drawn from the patient's opposite, noninfused arm. On day 183 (Week 26), the T sample is consideered a Randomized-Controlled Period assessment and the P sample is considered an Extension Period assessment. All collection times are recorded in eCRF. In the event of Clinical Deterioration, a blood sample for serum PK/PD and ADA analyses are collected if supplemental dosing is described herein.
¹²Patients are given aSafety Information Card prior to the first dose of study drug. At each visit throughout the study, staff ensures that the patient has the Patient Safety Information Card.

6. Standard Protocol Definitions

TABLE 12
Abbreviations and definitions for the study and follow-up period

| Abbreviation or | |
|---|---|
| Specialist Term | Explanation |
| Ab | Antibody |
| AChR | Acetylcholine receptor |
| AE | Adverse event |
| aHUS | Atypical hemolytic uremic syndrome |
| ANCOVA | Analysis of covariance |
| AZA | Azathioprine |
| BP | Blood Pressure |
| C5 | Complement protein 5 |
| CMAX | Maximal concentration |
| CMIN | Minimal concentration |
| eCRF | Electronic Case Report Form |
| C-SSRS | Columbia-Suicide Severity Rating Scale |
| ECG | Electrocardiogram |
| EDC | Electronic Data Capture |
| EIU | Exposure in-utero |
| EOI | Event of Interest |
| EOS | End of Study |
| EQ-5D | EuroQoL |
| ET | Early Termination |
| EU | European Union |
| FAS | Full Analysis Set |
| FVC | Forced Vital Capacity |
| GCP | Good Clinical Practices |
| gMG | Generalized Myasthenia Gravis |
| HAHA | Human Anti-human Antibody |
| HCG | human chorionic gonadotropin |
| HR | Heart Rate |
| IB | Investigator Brochure |
| ICF | Informed Consent Form |
| ICH | International Conference on Harmonization |
| ICU | Intensive Care Unit |
| IEC | Independent Ethics Committee |
| IVIg | Intravenous Iminunoglobulin G |
| IP | Investigational Product |
| IRB | Institutional Review Board |
| IST | Immunosuppressant Therapy |
| IV | Intravenous |
| IVIg | Intravenous immunoglobulin |
| IXRS | Interactive voice or web response system |
| mAb | Monoclonal Antibody |
| MedDRA | Medical Dictionary for Regulatory Activities |
| MG | Myasthenia Gravis |
| MG-ADL | MG activity of daily living profile |
| MGC | Myasthenia Gravis Composite |
| MGFA | Myasthenia Gravis Foundation of America |
| MM | Minimal manifestation |
| MMF | Mycophenolate Mofetil |
| MMT | Manual Muscle Test |
| MTX | Methotrexate |
| MuSK | Muscle-specific tyrosine kinase |
| NIF | Negative inspiratory force |
| NMJ | Neuromuscular junction |
| oMG | Ocular Myasthenia Gravis |
| PD | Pharmacodynamics |
| PE | Plasmapheresis or Plasma Exchange |
| PI | Principal Investigator |
| PIS | Post-Intervention Status |
| PK | Pharmacokinetics |
| PNH | Paroxysmal Nocturnal Hemoglobinuria |
| PP | Per-Protocol Population |
| QOL | Quality Of Life |
| QMG | Quantitative Myasthenia Gravis |
| RR | Respiration Rate |
| RSI | Reference Safety Information |
| SAE | Serious Adverse Event |
| SAP | Statistical Analysis Plan |
| SFEMG | single-fiber electromyography |
| SOC | System Organ Class |
| TEAE | Treatment Emergent Adverse Events |
| TESAE | Treatment Emergent SAE |
| US | United States |
| VAS | Visual Analog Scale |
| WHODrug | World Health Organization Drug Dictionary |

Clinical Deterioration

For this protocol, Clinical Deterioration is defined as follows:

1. Patients who experience an MGCrisis, which is defined as weakness from MG that is severe enough to necessitate intubation or todelay extubation following surgery. The respiratory failure is due to weakness of respiratory muscles. Severe bulbar (oropharyngeal) muscle weakness often accompanies the respiratory muscle weakness, or may be the predominant feature in some patients, or,

2. Significant symptomatic worsening to ascore of 3 or a2-point worsening from Baseline on any one of the individual MG-Activities of Daily Living (MG-ADL) items other than double vision or eyelid droop, or,

3. Administration of rescue therapy to apatient whose, in the opinion of the Investigator or Investigator-designated physician, health would be injeopardy, if rescue therapy were not given (e.g, emergent situations).

Unscheduled Visits

Under exceptional circumstances, additional (unscheduled) visits outside the specified visits are permitted at the discretion of the Investigator. Procedures, tests, and assessments is performed at the discretion of the Investigator and efforts are made to map the corresponding data to the appropriate visit.

Properly Trained Clinical Evaluator

Properly Trained Clinical Evaluators are study staff who have been certified in administering the MG-ADL, QMG and MGC assessments. Only Properly Trained Clinical Evaluators administer these assessments. A Properly Trained Clinical Evaluator is a neurologist, physical therapist, or other study team member delegated by the Investigator. Only the Investigator or a neurologist performs the manual muscle test (MMT), components of the MGC, the MGFA-PIS, and Myasthenia Gravis Foundation of America (MGFA) Classification. Clinical Evaluator training and certification for this protocol takes place either at the Investigator's Meeting or via the Sponsor's designated on-line training portal.

Responsibilities for Myasthenia Gravis Assessments

Responsibilities for MG assessments are listed in Table 13. Throughout the study, MG assessments are performed at approximately the same time of day by a Properly Trained Clinical Evaluator, and preferably the same evaluator.

TABLE 13
MG assessments and responsibilities

| Assessment | Evaluator |
|---|---|
| MG-ADL | Properly Trained Clinical Evaluator |
| QMG | Properly Trained Clinical Evaluator |
| MGC | Propedy Trained Clinical Evaluator |
| MGC (MMT Components) | Investigator or Neurologist |
| MGFA-PIS | Investigator or Neurologist |
| MGFA Classification | Investigator or Neurologist |

Abbreviations: MG-ADL= Myasthenia Gravis Activities of Daily Living Profile; MGC = Myasthenia Gravis Composite scale; MGFA = Myasthenia Gravis Foundation of America; MGFA-PIS = Myasthenia Gravis Foundation of America Post-Intervention Status; MMT = manual muscle test; QMG = Quantitative Myasthenia Gravis score for disease severity.

Scientific Rationale for Study Design

Published data support the MG-ADL profile as an established, sensitive, and objective assessment of treatment response over time in patients with gMG (Howard, J. et al., Muscle Nerve, 56:328-30, 2016).

The safety parameters being evaluated are commonly used in clinical studies per International Council for Harmonisation of Technical Requirements for Pharmaceuticals for Human Use (ICH) and Good Clinical Practice (GCP) guidance.

Placebo is selected as the control and patients are allowed to continue stable therapy with standard of care therapy (e.g., ISTs) throughout the course of the study, which thereby allows for comparison of the safety and efficacy of ravulizumab when administered in addition to the patient's standard of care treatment to current standard of care therapies in patients with gMG.

Given the heterogeneity of the disease and fluctuation in the severity of symptoms, there is no single international standard of care accepted, and targeted treatment with complement inhibitor drugs, such as the recently introduced eculizumab, is not yet widely available to patients worldwide and is not yet considered standard of care for all patients with gMG. A placebo-controlled study allows for the evaluation of treatment effect and allows for a double-blind design; an important study condition to be maintained when considering endpoints that includes neurological scales, which are known to be especially prone to placebo effects. The placebo-controlled part of the study is limited to 26 weeks, after which time all patients transition to open-label treatment with ravulizumab for up to 2 years during the OLE Period. At all points throughout the study, physicians are encouraged to prioritize patient safety, and if patients experience Clinical Deterioration, the full range of rescue therapies are permitted.

Justification for Dose

Ravulizumab is currently being studied in Phase 3 clinical studies in patients with PNH and aHUS, with PK/PD data extensively collected from all studies. Ravulizumab dosage regimens for these indications are selected based on comprehensive modeling and simulation analyses of the Phase 1 and 2 PK/PD data in healthy volunteers and PK/PD/efficacy (lactate dehydrogenase) and safety data in patients with PNH, and are considered optimal for achieving immediate, complete, sustained inhibition of terminal complement activity within each dosing interval and for the entire treatment course in all patients. The Phase 3 body weight-based dosage regimen (Table 14) are tested in patients with gMG in the current study.

TABLE 14
Ravulizumab weight-based dosing

| | | Maintenance Dose (mg) |
|---|---|---|
| Weight (kg) | Loading Dose (mg) | (administered q8w) |
| ≥40 to <60 | 2400 | 3000 |
| ≥60 to <100 | 2700 | 3300 |
| ≥100 | 3000 | 3600 |

Abbreviation: q8w = every 8 weeks.

Consistent with approved eculizumab labeling for treating adult and pediatric patients with aHUS and adult patients with gMG, supplemental dosing of ravulizumab in the amount of 50% (rounded up if not in integral of 300 mg due to vial configuration) is given in the setting of concomitant PP/PE rescue therapy and. For adult patients with gMG, supplemental dosing of ravulizumab (in the amount of 600 mg) is given in the setting of concomitant IVIg rescue therapy. The 600 mg per week supplemental ravulizumab dose is selected based on PK simulations considering the published data describing the impact of co-administration of IVIg on eculizumab PK/PD (Table 1; Table 2; Fitzpatrick, A. et al., J. Peripher. Nerv. Syst., 16:84-91, 2011).

Supplemental study drug (or placebo) dosing is required if PE/PP or IVIg rescue therapy is provided on non-dosing days; no supplemental study drug (or placebo) dosing is required if PE/PP or IVIg infusion is provided on a dosing day, but it occurs prior to study drug administration. If PE/PP or IVIg is administered on scheduled dosing visits, regular dosing is followed 60 minutes after the completion of PE/PP or IVIg. If PE/PP or IVIg is administered on non-scheduled dosing visits, for patients receiving PE/PP: supplemental dose is administered 4 hours after the PE/PP session is completed; for patients receiving IVIg: supplemental dose is administered 4 hours after the last continuous session(s) of IV Ig is completed as described herein.

The favorable benefit/risk profiles of ravulizumab from the recently completed Phase 3 studies in patients with PNH confirm immediate (after the first dose or loading dose), complete (free C5<0.5 μg/mL) and sustained (throughout entire active treatment course) terminal complement inhibition under the above investigated dosage regimen.

After the 26-Week Randomized-Controlled Period and assessments on Day 183 (Week 26), patients in the placebo group receive a blinded loading dose of ravulizumab and patients in the ravulizumab group receive a blinded ravulizumab dose of 900 mg; the 900 mg dose is chosen to ensure maintenance of complete C5 inhibition until the next scheduled maintenance dose at Week 28 (Day 197). Starting at Week 28 (Day 197), all patients begin open-label ravulizumab maintenance doses q8w.

The proposed q8w dosage regimen facilitates studying a range of PK drug exposures useful in assessing ravulizumab exposure-response relationships in patients with gMG. Safety and tolerability of ravulizumab have been established over a wide range of PK exposures, including those expected under the proposed gMG dosage regimens, in healthy volunteers and patients.

End of Study Definition

A patient is considered to have completed the study if:
The patient has completed all periods of the study including the last visit of the OLE Period, or
In the event the study is completed early, the patient has completed all applicable periods of the study including the EOS visit
The patient completes the study early (and completes the EOS Visit) because the study drug has become registered or approved (in accordance with country-specific regulations)

Measurement of the primary endpoints is complete after the last visit of the last patient in the Randomized-Controlled Period. The EOS is defined as the date of the last visit of the last patient in the study or last scheduled procedure shown in the schedule of activities (see, Table 10 and Table 11) for the last patient in the study globally. The study completion date corresponds to the last visit when the final patient in the study is examined or received an intervention for the primary or secondary endpoints and AEs.

7. Study Population

Prospective approval of protocol deviations to recruitment and enrollment criteria, also known as protocol waivers are not allowed.

### Inclusion Criteria

Patients are eligible to be included in the study only if all of the following criteria apply:

Age

1. Male and female patients are aged ≥18 years of age at the time of signing the informed consent

Type of Patient and Disease Characteristics

2. Diagnosed with MG at least 6 months (180 days) prior to the date of the Screening Visit, as confirmed by protocol-specific criteria (see below).

3. Diagnosis of MG is made by the following tests:
a. Positive serologic test for anti-AChR Abs as confirmed at screening, and
b. One of the following:
   History of abnormal neuromuscular transmission test demonstrated by single-fiber electromyography or repetitive nerve stimulation;
   History of positive anticholinesterase test (e.g., edrophonium chloride test);
   Demonstrated improvement in MG signs on oral cholinesterase inhibitors, as assessed by the treating physician.

4. Myasthenia Gravis Foundation of America Clinical Classification Class II to IV at screening.

5. MG-ADL profile is ≥6 at screening and randomization (Day 1).

6. Patients receiving treatment with any of the following are receiving treatment and on a stable dose for the time periods specified below prior to the date of the Screening Visit:
Azathioprine (AZA): is on AZA for ≥6 months (180 days) and have been on a stable dose for ≥2 months (60 days);
Immunosuppressive therapies (e.g., mycophenolate mofetil [MMF], methotrexate [MTX], cyclosporine [CYC], tacrolimus [TAC], or cyclophosphamide [CY]), are on the IST for ≥3 months (90 days) and are on a stable dose for ≥1 month (30 days);
Oral corticosteroids, are on a stable dose for ≥4 weeks (28 days);
A cholinesterase inhibitor, at the time of the Screening Visit, are on a stable dose for ≥2 weeks (14 days).

7. To reduce the risk of meningococcal infection (N. meningitidis), all patients are vaccinated against meningococcal infections within the 3 years prior to, or at the time of, initiating study drug. Patients who initiate study drug treatment less than 2 weeks after receiving a meningococcal vaccine receive treatment with appropriate prophylactic antibiotics until 2 weeks after vaccination.

Weight

8. Body weight ≥40 kg at the time of screening.

Pregnancy and Contraception

9. Patients of childbearing potential and patients with partners of childbearing potential use contraception for avoiding pregnancy while on treatment and for 8 months after last dose of study drug.

Informed Consent

10. Capable of giving signed informed consent. As part of the informed consent:
The Investigator or his/her representative explains the nature of the study to the patient or his/her legally authorized representative and answers all questions regarding the study.
Patients are informed that their participation is voluntary. Patients or their legally authorized representative are required to sign a statement of informed consent that meets the requirements of 21 CFR 50, local regulations, ICH guidelines, Health Insurance Portability and Accountability Act requirements, where applicable, and the IRB/IEC or study center.
The medical record includes a statement that written informed consent was obtained before the patient was enrolled in the study and the date the written consent was obtained. The authorized person obtaining the informed consent also signs the ICF.
Patients is reconsented to the most current version of the informed consent forms (ICF(s)) during their participation in the study. A copy of the ICF(s) is provided to the patient.
The Investigator retains the original version of the signed ICF(s). A copy of the signed ICF(s) is provided to the patient.
A patient who is rescreened is not required to sign another ICF unless an updated ICF is available.

Exclusion Criteria

Patients are excluded from the study if any of the following criteria apply:

Medical Conditions
1. Any active or untreated thymoma. History of thymic carcinoma or thymic malignancy unless deemed cured by adequate treatment with no evidence of recurrence for ≥5 years before Screening;
2. History of thymectomy within the 12 months prior to screening;
3. History of hypersensitivity to any ingredient contained in the study drug, including hypersensitivity to murine proteins;
4. History of N. meningitidis infection;
5. Human immunodeficiency virus (HIV) infection (evidenced by HIV-1 or HIV-2 antibody titer);
6. Known medical or psychological condition(s) or risk factor that, in the opinion of the Investigator, interfered with the patient's full participation in the study, poses any additional risk for the patient, or confounds the assessment of the patient or outcome of the study;
7. History of hospitalization for ≥24 hours, for any reason, within the 4 weeks (28 days) prior to screening;
8. Clinical features that, in the opinion of the Investigator, are consistent with MG crisis/exacerbation or Clinical Deterioration, at the time of the Screening Visit or at any time prior to randomization;
9. Female patients who plan to become pregnant or are currently pregnant or breastfeeding;
10. Female patients who have a positive pregnancy test result at screening or on Day 1. Prior/Concomitant Therapy
11. Use of the following within the time period specified below:
   IVIg within the 4 weeks (28 days) prior to randomization (Day 1);
   Use of PE within the 4 weeks (28 days) prior to randomization (Day 1);
   Use of rituximab within the 6 months (180 days) prior to screening.
12. Patients who have received previous treatment with complement-inhibitors (e.g., eculizumab).

Prior/Concurrent Clinical Study Experience
13. Participation in another interventional treatment study or use of any experimental therapy within 30 days before initiation of study drug on Day 1 in this study or within 5 half-lives of the study drug, whichever is greater.

Screen Failures

Screen failures are defined as patients who consent to participate in the clinical study but are not subsequently randomized to a treatment group. A minimal set of screen failure information is required to ensure transparent reporting of screen failure patients to meet the Consolidated Standards of Reporting Trials publishing requirements and to respond to queries from regulatory authorities. Minimal information includes demography, screen failure details, eligibility criteria, and any serious adverse event (SAE).

Individuals who do not meet the criteria for participation in this study (screen failure) may be rescreened once based on discussion and agreement between the Investigator and the Medical Monitor.

A patient who experiences a gMG Clinical Deterioration or exacerbation/crisis during the Screening Period will be considered a screening failure. Such patients may be rescreened with Sponsor approval once they are treated and medically stable, in the opinion of the Investigator. At least 28 days of clinical stability must exist prior to enrollment. The patient must meet all of the inclusion criteria and none of the exclusion criteria at the time of rescreening to enter the study.

8. Study Drug

### Study Drugs Administered

Ravulizumab is formulated at pH 7.0 and is supplied in 30 mL single-use vials. Each vial of ravulizumab contains 300 mg of ravulizumab (10 mg/mL) in 10 mM sodium phosphate, 150 mM sodium chloride, 0.02% polysorbate 80, and water for injection. The comparator product is formulated as a matching sterile, clear, colorless solution with the same buffer components, but without active ingredient. Additional details are presented in Table 15.

TABLE 15
Study drug administered

| Product Name | Ravulizumab | Placebo |
|---|---|---|
| Dosage Form | Concentrated sterile, preservative-free | Sterile, preservative-free aqueous |
| | aqueous solution (10 mg/mL) in single- | solution in single-use 30 mL vials |
| | use 30 mL vials | |
| Route of Administration | Intravenous infusion | Intravenous infusion |
| Dosing Instructions | Refer to pharmacy manual for dosing | Refer to pharmacy manual for dosing |
| | instructions | instructions |
| Packaging and Labeling | Glass vials and stoppered with a butyl | Glass vials and stoppered with a butyl |
| | rubber stopper with an aluminum | rubber stopper with an aluminum |
| | overseal and a flip-off cap. Study drag | overseal and a flip-off cap. Study drug |
| | will be supplied in kits. | will be supplied in kits. |
| Physical Description | Liquid solution practically free from | Liquid solution practically free from |
| | particles | particles |
| Manufacturer | Alexion Pharmaceuticals, Inc. or | Alexion Pharmaceuticals, Inc. or |
| | Contracted Manufacturing | Contracted Manufacturing |
| | Organization | Organization |

Source: product specifications

Study drug is administered as indicated in Table 16.

During the Randomized-Controlled Period, patients in the ravulizumab or placebo treatment groups receive a weight-based loading dose of ravulizumab or placebo, respectively, on Day 1 (Visit 2). At Visit 4 (Week 2), patients in the ravulizumab or placebo treatment groups receive weight-based maintenance doses or ravulizumab or placebo, respectively, q8w through the completion of the Randomized-Controlled Period (see, Table 16). After the completion of the Randomized-Controlled Period, patients enter the OLE Period.

After the 26-Week Randomized-Controlled Period and assessments on Day 183 (Week 26), patients in the placebo group receive a blinded loading dose of ravulizumab and patients in the ravulizumab group receive a blinded ravulizumab dose of 900 mg; the 900 mg dose is chosen to ensure maintenance of complete C5 inhibition until the next scheduled maintenance dose at Week 28 (Day 197). Starting at Week 28, all patients begin open-label ravulizumab maintenance doses q8w.

TABLE 16
Reference chart for weight-based dosing

| | | | | | | | |
|---|---|---|---|---|---|---|---|
| | | | | | | Diluent | |
| | | | | | | (0.9% | |
| | | | | | | Sodium | |
| | | Body | Ravulizumab | Ravulizumab | Placebo | Chloride) | Total |
| | Ravulizumab or | Weight | Dose | Volume | Volume | Volume | Volume |
| Study Period | Placebo Dosing | (kg)¹ | (mg) | (mL) | (mL) | (mL) | (mL) |

Ravulizumab Group

| | | | | | | | |
|---|---|---|---|---|---|---|---|
| Randomized- | Loading dose | ≥40 to <60 | 2400 | 240 | 0 | 240 | 450 |
| Controlled | (Day 1) | ≥60 to <100 | 2700 | 270 | 0 | 270 | 540 |
| | | ≥100 | 3000 | 300 | 0 | 300 | 600 |
| | Maintenance | ≥40 to <60 | 3000 | 300 | 0 | 300 | 600 |
| | dose | ≥60 to <100 | 3300 | 330 | 0 | 330 | 660 |
| | (Days 15, 71, 127) | ≥100 | 3600 | 360 | 0 | 360 | 720 |
| Open-Label | Blinded Dose² | ≥40 to <60 | 900 | 90 | 150 | 240 | 480 |
| Extension | (Day 183) | ≥60 to <100 | 900 | 90 | 180 | 270 | 540 |
| | | ≥100 | 900 | 90 | 210 | 300 | 600 |
| | Open-label maintenance | ≥40 to <60 | 300 | 300 | 0 | 300 | 600 |
| | dose | ≥60 to <100 | 3300 | 330 | 0 | 330 | 660 |
| | (Days 197 to 869 q8w) | ≥100 | 3600 | 360 | 0 | 360 | 720 |

Placebo Group

| | | | | | | | |
|---|---|---|---|---|---|---|---|
| Randomized- | Loading dose | ≥40 to <60 | 0 | 0 | 240 | 240 | 480 |
| Controlled | (Day 1) | ≥60 to <100 | 0 | 0 | 270 | 270 | 540 |
| | | ≥100 | 0 | 0 | 300 | 300 | 600 |
| | Maintenance | ≥40 to <60 | 0 | 0 | 300 | 300 | 600 |
| | dose | ≥60 to <100 | 0 | 0 | 330 | 330 | 660 |
| | (Days 15, 71, 127) | ≥100 | 0 | 0 | 360 | 360 | 720 |
| Open-Label | Blinded Dose [symbol] | ≥40 to <60 | 2400 | 240 | 0 | 240 | 480 |
| Extension | (Day 183) | ≥60 to <100 | 2700 | 270 | 0 | 270 | 540 |
| | | ≥100 | 3000 | 300 | 0 | 300 | 600 |
| | Open-label maintenance | ≥40 to <60 | 3000 | 300 | 0 | 300 | 600 |
| | dose | ≥60 to <100 | 3300 | 330 | 0 | 330 | 660 |
| | (Days 197 to 869 q8w) | ≥100 | 3600 | 360 | 0 | 360 | 720 |

¹Dose regimen is based on the patient's most recently recorded body weight from a previous study/screening visit.
²Blinded dose on Day 183 (Week 26) for patients who were randomized to the ravulizumab group and are entering into the Open-Label Extension Period. [symbol]
indicates data missing or illegible when filed

### Preparation/Handling/Storage/Accountability

Study drug is released to the site upon receipt of all required essential documents based upon federal, state, and local regulations.

Only patients enrolled in the study receive study drug and only authorized site staff supply or administer study drug. All study drug is stored in a secure, environmentally controlled, and monitored (manual or automated) area in accordance with the labeled storage conditions with access limited to the Investigator and authorized site staff.

Study Drug Preparation

Study drug is prepared and administered by a trained member of the site study team. Study drug is administered only to enrolled patients who are confirmed eligible for participation.

Preparation of ravulizumab and placebo doses is performed in accordance with study center-specific local standards by qualified and study-trained pharmacy personnel.

The handling and preparation of materials used to prepare and administer the study drug are carried out using aseptic techniques for sterile products.

All study patients, investigative-site personnel, Sponsor staff, Sponsor designees, and all staff directly associated with the conduct of the study are blinded to patient treatment assignments.

Further details on preparation and dose administration of study drug, as well as disposal of study drug, are found in the pharmacy manual.

Storage

The Investigator or designee confirms appropriate temperature conditions are maintained during transit for all study drugs received and that any discrepancies are reported and resolved before use of the study drug.

Upon arrival at the investigative site, the study drug is promptly removed from the shipping cooler and stored in refrigerated conditions at 2° C. to 8° C. (36° F. to 46° F.). The pharmacist immediately records the receipt of the study drug and notifies the distributor if vials are damaged and/or if temperature excursions have occurred during transportation. Study drug is stored in a secure, limited-access storage area and temperature is monitored daily.

Diluted solutions of study drug are stored at 2° C. to 8° C. (36° F. to 46° F.) for up to 24 hours prior to administration. The solution is allowed to warm to room temperature prior to administration.

The admixed drug product is at room temperature prior to administration. The material is not heated (e.g., by using a microwave or other heat source) other than by ambient air temperature.

Packaging and Labeling

The primary packaging of ravulizumab consists of a 30 mL vial (Type I borosilicate glass) with a stopper and a seal. The secondary packaging consists of a single vial carton. Both primary (vial) and secondary (carton) packaging include a booklet label with relevant information. Additional details are presented in Table 13 and in the pharmacy manual. The placebo has an identical appearance to that of ravulizumab.

Accountability

When a drug shipment is received at the site, the pharmacist verifies the contents, signs the packing invoice provided with the shipment, and maintains the original copy for review by the site monitor in the pharmacy binder. Additionally, study drug receipt (as well as condition of the study drug at the time of receipt) is reported to the IRT system to allow drug randomization, resupply, estimations, and drug expiration control.

Unless notified otherwise, empty vials and vials with residual materials are kept for inspection and accountability by the study monitor prior to their destruction or handled per local pharmacy standard operating procedures for clinical study drugs. Destruction of used and unused vials, either locally or centrally, are properly documented. Drug accountability is managed through the IRT system and detailed instructions on managing the IRT drug accountability module is included in the IRT User Guide. The IRT module performs accountability in two stages, where site personnel complete an initial accountability entry in the system followed by confirmation by the Study Monitor that the site correctly enters the appropriate status for all study drug. The pharmacist or designee maintains accurate records demonstrating dates and amount of study drug received, to whom dispensed (patient-by-patient accounting), and accounts of any study drug accidentally or deliberately destroyed. These drug accountability records are readily available upon request, and are reviewed throughout the study.

Each kit has a label and a place for the pharmacist to record the patient number and initials.

The study monitor examines the inventory during the study. Additionally, the inventory records are readily available to regulatory authorities, the local regulatory agency, or an independent auditor's inspection at any time.

Refer to the Pharmacy Manual for additional information.

Handling and Disposal

All clinical study material that is provided to the Investigator is stored in a secure place, and appropriately trained personnel allocate and dispense it. Detailed records of the amounts of the study drug received, dispensed, and destroyed are maintained.

To satisfy regulatory requirements regarding drug accountability, all remaining ravulizumab inventory is reconciled and destroyed or returned to Alexion at the end of the study according to applicable regulations.

Refer to the Pharmacy Manual for further information.

Randomization

Patients are randomized on Day 1 after the Investigator has verified that they are eligible. Patients are stratified by region (North America, Europe, Asia-Pacific, and Japan) and randomized 1:1 either to ravulizumab IV infusion or to placebo IV infusion. Patients are centrally randomized using IRT.

Blinding

All investigative site personnel, Sponsor staff, Sponsor designees, staff directly associated with the conduct of the study, and all patients are blinded to patient treatment assignments. The double-blind is maintained by using identical study drug kits and labels for ravulizumab and placebo. The placebo has an identical appearance to that of ravulizumab. The random code is maintained by the IRT provider. After the 26-Week Randomized-Controlled Period and assessments on Day 183 (Week 26), patients in the placebo group receive a blinded loading dose of ravulizumab and patients in the ravulizumab group receive a blinded ravulizumab dose of 900 mg. Starting at Week 28, all patients begin open-label ravulizumab maintenance doses q8w. For patients in the ravulizumab group, a blinded ravulizumab dose of 900 mg is chosen to ensure maintenance of complete C5 inhibition until the next scheduled maintenance dose at Week 28 (Day 197).

Unblinding should only be considered for the safety of the patient. If unblinding is deemed necessary by the Investigator, the Investigator makes a reasonable attempt to contact the Sponsor to discuss possible unblinding. After a reasonable attempt has been made, the Investigator unblinds the patient's treatment allocation using an IRT. The Investigator notes the date, time, and reason for unblinding. The Investigator also informs the Medical Monitor that the patient is unblinded; however, they do not reveal to the Medical Monitor the patients' treatment allocation.

When an adverse event (AE) is an unexpected or related and serious, the blind is broken for that specific patient only. The blind is maintained for persons responsible for the ongoing conduct of the study (such as the management, monitors, Investigators, etc.) and those responsible for data analysis and interpretation of results at the conclusion of the study, such as biometrics personnel. Unblinded information is only accessible to those who need to be involved in the safety reporting to Health Authorities, Independent Ethics Committees (IECs), and/or Institutional Review Boards (IRBs).

Any patient who is unblinded during the study is discontinued from the study.

Investigators receive only blinded information unless unblinded information is judged necessary for safety reasons.

### Concomitant Therapy

Prior medications (including vitamins and herbal preparations), including those discussed in the exclusion criteria and procedures (any therapeutic intervention, such as surgery/biopsy or physical therapy) the patient takes or undergoes within 28 days prior to the start of screening until the first dose of study drug, are recorded. In addition, history of meningococcal vaccination is collected for the 3 years prior to first dose of study drug.

All medication use and procedures undertaken during the study are recorded. This includes all prescription drugs, herbal products, vitamins, minerals, over-the-counter medications, and any other current medications. Concomitant medications are recorded from the first infusion of study drug through 8 weeks after the patient's last dose of study drug. Any changes in concomitant medications also are recorded. Any concomitant medication deemed necessary for the patient's standard of care during the study, or for the treatment of any AE, along with any other medications, other than those listed as prohibited medications as defined herein, are given at the discretion of the Investigator. However, it is the responsibility of the Investigator to ensure that details regarding all medications are recorded.

### Study Drug Compliance

Study drug is administered in a controlled setting under the supervision of the Investigator or designee, thereby ensuring compliance with study drug administration.

Palliative and Supportive Care

Palliative and supportive care is permitted during the course of the study for underlying conditions.

Allowed Medications

The medications described in the following sections are allowed under certain circumstances and restrictions.

Cholinesterase Inhibitors

For patients who enter the study receiving a cholinesterase inhibitor at screening, the dose and schedule of their cholinesterase inhibitor is maintained stable throughout the entire Randomized-Controlled and OLE Periods, unless there is compelling medical need. Increases in cholinesterase therapy that are required as a result of intercurrent illness or other medical cause of deterioration are permitted, but dosing is returned to dosing levels at study entry as soon as feasible and the Sponsor is notified of the change.
1. Cholinesterase inhibitor treatment is withheld for at least 10 hours prior to administration of the QMG and MGC tests.
2. If a decrease in cholinesterase inhibitor is considered based on clinical evaluation, Sponsor approval is obtained prior to the change in dose for the patient to remain on study.

Immunosuppressive Agents

The following immunosuppressive agents are allowed during the study: corticosteroid, AZA, MMF, MTX, TAC, CYC or CY. The immunosuppressive agent(s) and its appropriate dose level to be used for an individual patient is at the discretion of the treating physician/Investigator.
1. Corticosteroid: for patients who enter the study receiving oral corticosteroid, e.g., prednisone, the dose/schedule is not changed during the entire double-blind study period (i.e., the Randomized-Controlled Period). If a decrease or taper in steroid dose is considered during the Randomized-Controlled Period based on clinical evaluation, Sponsor approval is obtained prior to the change for the patient to remain on study. If the dose level subsequently is increased, the dose level increase is not above the dose level reported at the baseline (at the start of randomized treatment).
2. High-dose steroid is reserved for patients that experience clinical deterioration as defined herein. Every effort is made to notify the Sponsor within 24 hours of administration should a patient require rescue therapy for clinical deterioration.
3. AZA, MMF, MTX, TAC, CYC or CY: for patients who enter the study receiving above mentioned immunosuppressive agents, the dosing regimen of the immunosuppressive agent is not changed during the entire Randomized-Controlled Period. If a change in the dosing regimen is considered due to known toxicity or side effects associated with the given immunosuppressive agent, Sponsor approval is obtained prior to the dose change for the patient to remain on the study. A different immunosuppressive agent is not added or substituted during the 26-week Randomized-Controlled Period.

Plasma Exchange/Plasmapheresis/Intravenous Immunoglobulin

Use of PE/PP or IVIg is allowed for patients who experience a clinical deterioration as defined herein. The rescue therapy used for a particular patient is at the discretion of the Investigator. Every effort is made to notify the Sponsor within 24 hours should a patient require rescue therapy.

Supplemental study drug (or placebo) dosing is required if PE/PP or IVIg rescue therapy is provided on nondosing days; if PE/PP or IVIg infusion is provided on a dosing day, it must occur prior to study drug administration.
1. If PE/PP or IVIg is administered on nonscheduled dosing visits
   a. Patients receiving PE/PP: supplemental dose is administered 4 hours after the PE/PP session is completed
   b. Patients receiving IVIg: supplemental dose is administered 4 hours after the last continuous session(s) of IVIg is completed
   c. Supplemental dose amount may or may not vary depending on PE/PP or IVIg (Table 1 and Table 2)
2. If PE/PP or IVIg is administered on scheduled dosing visits,
   a. Regular dosing is followed 60 minutes after the completion of PE/PP or IVIg.
3. No gap is required between a supplemental dose and the regular scheduled dose.

### Disallowed Medications

The following concurrent medications are prohibited during the study:
Rituximab
Eculizumab (or other complement-inhibitors)

Patient use of rituximab or eculizumab (or other complement inhibitors) at any point during the study results in the patient being discontinued from the study.

### Rescue Therapy

Rescue therapy (e.g., high-dose corticosteroid, PP/PE or IVIg) is allowed when a patient's health is in jeopardy if rescue therapy is not administered (e.g., emergent situations) or, if a patient experiences clinical deterioration as defined herein. The rescue therapy used for a particular patient is at the discretion of the Investigator. The date and time of rescue medication administration as well as the name and dosage regimen of the rescue medication is recorded.

Should a patient require rescue therapy, every effort is made to notify the Sponsor within 24 hours.

Intervention after the end of the study

Patients return to the care of their treating physician at the completion of study participation.

9. Discontinuation of Study Intervention and Patient Discontinuation/Withdrawal Discontinuation of Study Intervention

A patient may withdraw from the study at any time at his/her own request, or may be withdrawn at any time at the discretion of the Investigator for safety, behavioral, compliance, or administrative reasons. If a patient discontinues treatment from the study, the Investigator attempts to perform (if the patient agrees) assessments specified for the ET Visit, or if not possible, a follow-up phone is conducted 8 weeks after the last dose of study drug is administered (Table 10 and Table 11). Attempts are also made to follow all patients for safety for a total of 8 weeks from the day the last dose of study drug is administered. The Sponsor and site monitor are notified as soon as possible. If a patient is withdrawn from the study or withdraws consent no further data are collected. Patients who withdraw from the study are not be replaced.

Patients are discontinued from study drug if any of the following occur during the study:

1. Serious hypersensitivity reaction (such as bronchospasm with wheezing or requiring ventilator support or symptomatic hypotension or serum sickness-like reactions manifesting 1 to 14 days after study drug administration;

2. Severe uncontrolled infection;

3. Pregnancy or planned pregnancy; or

4. Sponsor deems it is in the best interest of the patient.

5. Use of rituximab, eculizumab (or other complement-inhibitors)

The Investigator contacts the Medical Monitor prior to discontinuing a patient from study drug. If a patient discontinues from treatment, the patient is encouraged to return for the ET Visit (Table 10 and Table 11) 8 weeks after the patient's last dose of study drug.

The reason for the treatment discontinuation (e.g., patient withdraws consent, patient withdrawal from procedures, physician decision, AE, or other reason specified in eCRF) is recorded.

If a female patient is permanently discontinued from study drug due to pregnancy, the Investigator makes a reasonable attempt to follow-up, in accordance with local laws and regulations, until the outcome of the pregnancy is known.

If the patient withdraws consent for disclosure of future information, the Sponsor retains and continues to use all data collected before such a withdrawal of consent.

If a patient withdraws from the study, the patient may request destruction of any samples taken and not tested, and the Investigator documents this in the site study records as well as informs the site monitor and Sponsor.

Lost to Follow Up

A patient is considered lost to follow-up if the patient repeatedly fails to return for scheduled visits and is unable to be contacted by the study site.

The following actions must be taken if a patient fails to return to the clinic for a required study visit:

1. The site attempts to contact the patient and reschedule the missed visit as soon as possible and counsels the patient on the importance of maintaining the assigned visit schedule and ascertain whether or not the patient wishes to and/or should continue in the study.

2. Before a patient is deemed lost to follow up, the Investigator or designee makes every effort to regain contact with the patient (where possible, 3 telephone calls and, if necessary, a certified letter to the patient's last known mailing address or local equivalent methods). These contact attempts are documented in the patient's medical record.

3. Should the patient continue to be unreachable, the patient is considered to have withdrawn consent and future missed visits are not considered protocol deviations.

10. Study Assessments and Procedures

Efficacy Assessments

### Hospitalization

Information related to all-cause hospitalization is collected from patient signing of the ICF through the OLE Period. Hospitalizations are defined as all admissions to a healthcare facility, irrespective of the underlying relation to MG. Dates of admission/discharge, reasons for hospitalization, relationship to MG, and other relevant information are collected.

Hospitalization includes the following:
1. Emergency room visits related to MG with or without admission regardless of duration;
2. Unplanned admission to healthcare facility, regardless of relationship to MG;
3. Inpatient administration of MG-related infusion/treatment at a hospital facility (e.g., IVIg, PP, PE, ventilator support).

Hospitalization does not include the following:
1. Routine study drug administration;
2. Rehabilitation facility;
3. Hospice facility;
4. Nursing/assisted living/extended-care facility;
5. Outpatient-care facilities;
6. Planned admission for treatment of a pre-existing condition (i.e., condition that started prior to obtaining informed consent);
7. Planned/unplanned outpatient surgery (e.g., used as a surgical facility);
8. Emergency room visit unrelated to MG without admission;
9. Outpatient administration of infusion/treatment at a hospital facility (e.g., IVIg, PP).

### Clinical Deterioration

Information related to clinical deterioration, as defined herein, are collected from patient signing of the ICF through the OLE Period. The evaluation visit for a clinical deterioration is performed as soon as possible, within 48 hours of notification to the Investigator of the symptom onset. Additional Unscheduled Visits as defined herein, are scheduled at the discretion of the Investigator. The following tests and procedures are completed at this visit:
1. Measure vital signs and pulse oximetry, including assessments of systolic and blood pressure (BP), temperature (° C. or ° F.), oxygen saturation (SO2), and heart rate (HR).
2. Record any new medications or changes to concomitant medications, including all treatments for MG.
3. Evaluate and record any new AEs or changes in AEs since the previous visit.
4. Administer MG-ADL by a properly trained evaluator, preferably the same evaluator, throughout the study. The recall period is the preceding 7 days or since the last visit whichever occurs earlier.
5. Administer clinical assessments QMG and MGC; these are performed at approximately the same time of day by a properly trained evaluator, preferably the same evaluator, throughout the study.
6. Collect blood sample for the AChR auto-Abs test.
7. Collect blood samples for clinical laboratory tests (Table 17). The tests detailed in Table 17 are performed by the central laboratory. Protocol-specific requirements for inclusion or exclusion of patients are detailed herein. Additional tests are performed at any time during the study.
8. If medically indicated for evaluation of clinical deterioration, additional tests are performed at the discretion of the Investigator.
9. PK/PD sampling at or during clinical deterioration Visit:
   a. Collect 1 blood sample for PK and free C5 assays if no study drug is administered.
   b. If the study drug is administered at the clinical deterioration Visit, according to the protocol schedule, collect 2 blood samples, trough and peak, at [1] 5-90 minutes before the study drug infusion and [2] within the 30 minutes following completion of study drug infusion.
c. If a the patient receives PP/PE or IVIg at the time of Clinical Deterioration, a supplemental dose of study drug is administered. Collect blood samples for PK, and free C5 at [1]5-90 minutes before PP/PE or IVIg, [2] after PP/PE or IVIg and before study drug infusion, and [3] within the 30 minutes following completion of study drug infusion.

TABLE 17
Protocol-required safety laboratory assessments

| Laboratory | | | |
|---|---|---|---|
| Assessments | Parameters | | |
| Hematology | Platelet count | RBC indices: | WBC count with differential: |
| | RBC count | Distribution width | Neutrophils |
| | Hemoglobin | Mean corpuscular volume | Lymphocytes |
| | Hematocrit | Mean corpuscular | Monocytes |
| | | hemoglobin | Eosinophils |
| | | % Reticulocytes | Basophils |
| Clinical | BUN | AST/SGOT | Total and direct bilirubin |
| Chemistry | C-reative protein | ALT/SGPT | Total protein |
| | Creatinine | Alkaline phosphatase, | Albumin |
| | Chloride | Gamma glutamyltransferase | Uric acid |
| | Potassium | | |
| | Bicarbonate | | |
| | Sodium | | |
| | Glucose (nonfasting) | | |

| | |
|---|---|
| Coagulation | international normalized ratio, partial thromboplastin time, prothrombin time |
| Routine urinalysis | Appearance, color, specific gravity, pH, glucose, protein, creatinine, blood, ketones, |
| | bilirubin, urobilinogen, nitrite, Microscopic examination (if blood or protein is |
| | abnormal) |
| Other Screening | Serum/urine beta-hCG pregnancy test(as needed for patients of child-bearing potential) |
| tests | Serum follicle-stimulating hormone test (as needed for patients who consider themselves |
| | postmenopausal) |
| | HIV-1 and HIV-2 antibodies |
| | The results of each test must be entered into the eCRF. |
| Complement | Free C5 |
| activity | |

Abbreviations: ALT = alanine aminotransferase; AST = aspartate aminotransferase; BUN = blood urea nitrogen; C5 = complement component 5; eCRF = electronic case report form; hCG = human chorionic gonadotropin; HIV-1 = human immunodeficiency virus type 1; HIV-2 = human immunodeficiency virus type 2; RBC = red blood cells; SGOT = serum glutamic oxaloacetic transaminase; SGPT= serum glutamic pyruvic transaminase; WBC = white blood cells.

Safety Assessments

### Physical Examination

A physical examination includes assessments of the following organs/body systems: skin, head, ears, eyes, nose, throat, neck, lymph nodes, pulse, chest, heart, abdomen, extremities; musculoskeletal and general neurologic examination. An abbreviated physical examination consists of a body-system relevant examination based upon Investigator judgment and patient symptoms. For consistency, all efforts are made to have the physical examination performed by the same qualified study staff.

### Vital Signs and Pulse Oximetry

Vital signs and pulse oximetry are measured at every visit and include assessments of systolic and diastolic BP (mmHg), temperature (° C. or ° F.), SO2, and HR (beats per minute). Vital signs are obtained after the patient has been supine or seated for at least 5 minutes. Ideally, each patient's BP is measured using the same arm.

### Electrocardiogram

Single 12-lead electrocardiogram (ECG) are obtained as outlined in the schedule of activities (Table 10 and Table 11) using an ECG machine that automatically calculates the HR and measures PR, QRS, QT, and QTc intervals. Patients are supine for approximately 5-10 minutes before ECG collection and remain supine but awake during ECG collection.

The Investigator or designee are responsible for reviewing the ECG to assess whether the ECG is within normal limits and determine the clinical significance of the results.

### Clinical Safety Laboratory Assessments

Laboratory assessments are tested at a central laboratory facility. Any clinically significant abnormal results are followed until resolution or stabilization.

All protocol-required laboratory assessments, as defined herein are conducted in accordance with the laboratory manual and the schedule of activities (Table 10 and Table 11).

The Investigator reviews the laboratory report, documents this review, and records any clinically relevant changes occurring during the study. The laboratory reports are filed with the source documents.

Clinically significant abnormal laboratory findings associated with the underlying disease are not considered AEs unless they are judged by the Investigator to be more severe than expected for the patient's condition.

If such values do not return to normal/baseline within a period of time judged reasonable by the Investigator, the etiology is identified and the Sponsor notified.

### Urinalysis and Urine Chemistry

Urine samples are analyzed for the parameters listed in (Table 17). A microscopic examination of urine samples is performed if the results of the macroscopic analysis are abnormal.

Urine samples are also analyzed to measure protein and creatinine to calculate the urine protein:creatinine ratio.

### Virus Serology

Human immunodeficiency virus testing for HIV-1 and HIV-2 is required of all patients prior to enrollment. Patients who are HIV positive are not enrolled.

### Immunogenicity Assessments

Blood samples are collected to test for presence of ADAs to ravulizumab in serum prior to study drug administration. Further characterization of antibody responses are conducted as appropriate, including binding and neutralizing antibodies, PK/PD, safety, and activity of ravulizumab. Antibodies to ravulizumab are evaluated in serum samples collected from all patients according to the schedule of activities (Table 10 and Table 11). Serum samples are screened for antibodies binding to ravulizumab and the titer of confirmed positive samples are reported. The detection and characterization of antibodies to ravulizumab are performed using a validated assay by or under the supervision of the Sponsor.

### Suicidal Risk Monitoring

Columbia-Suicidal Severity Rating Scale

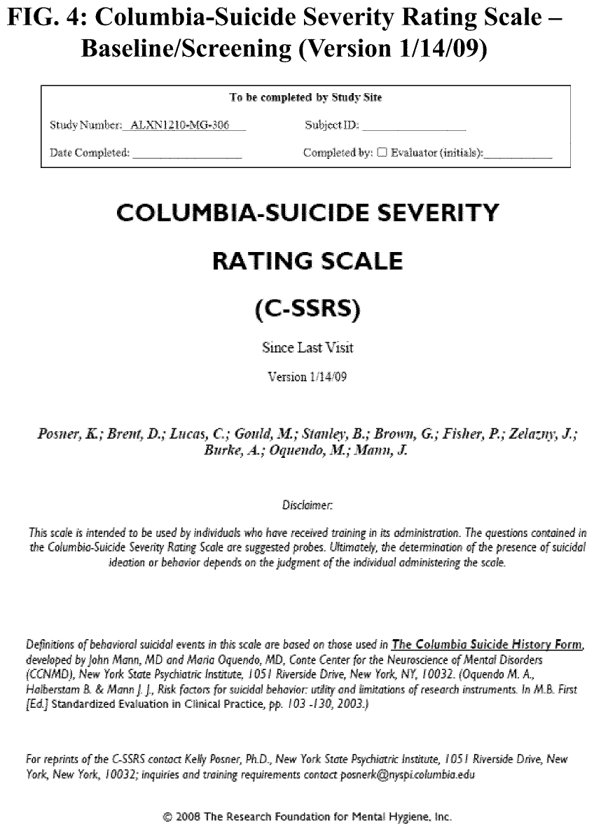

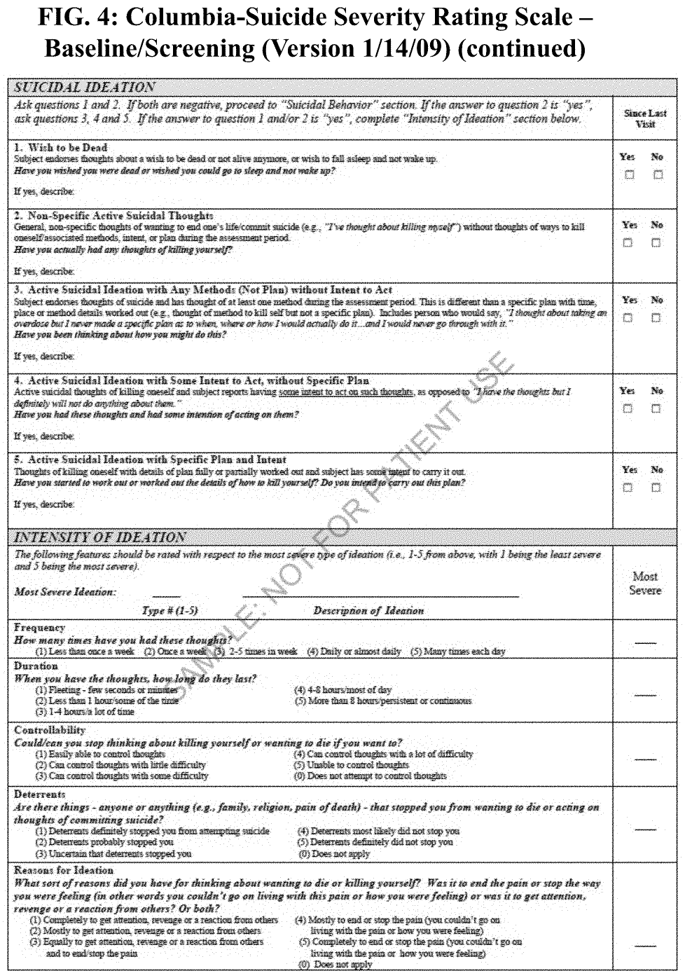

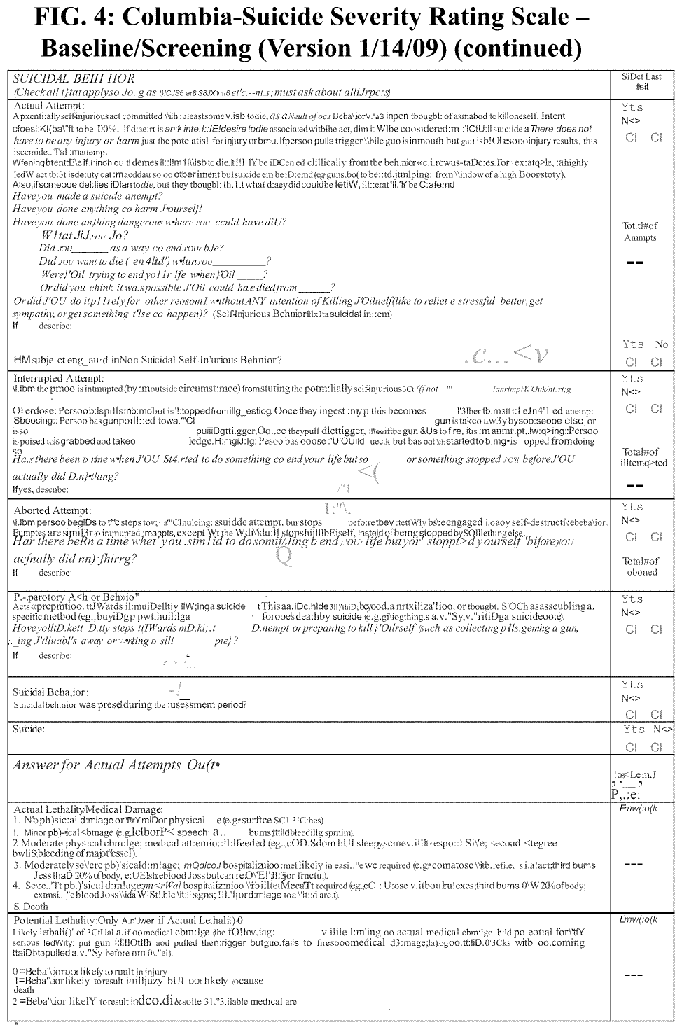

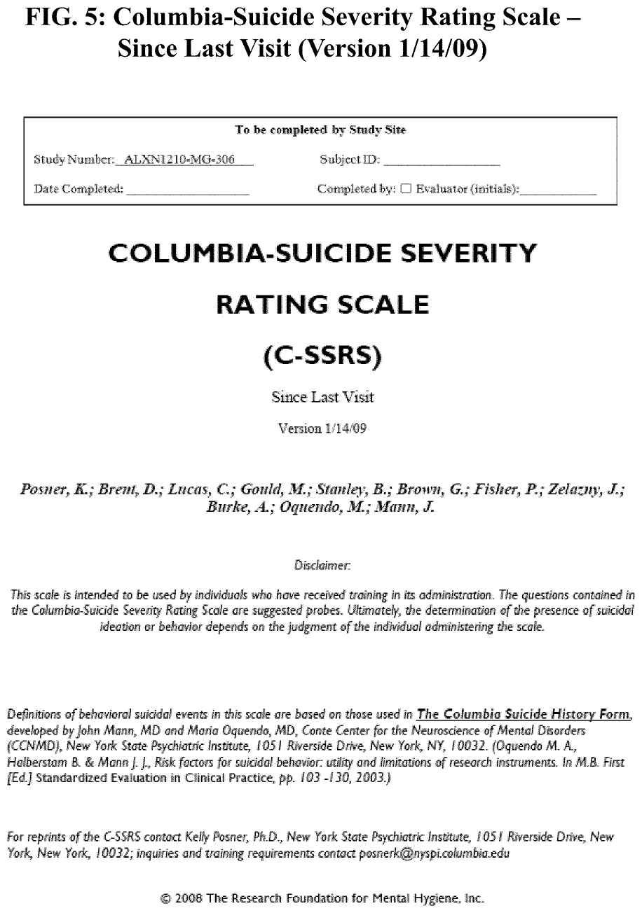

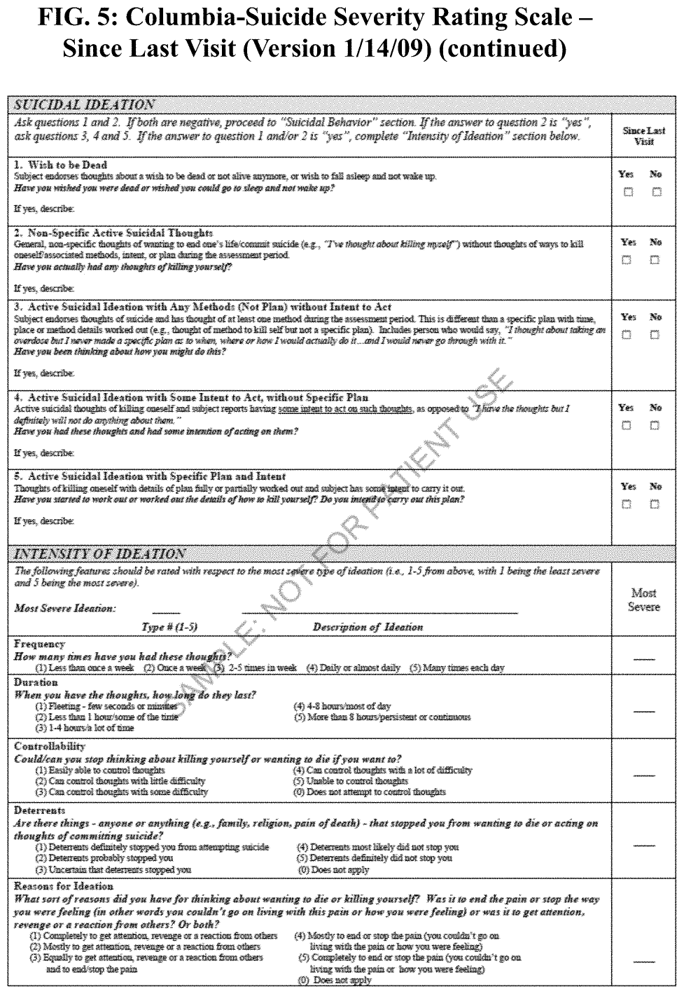

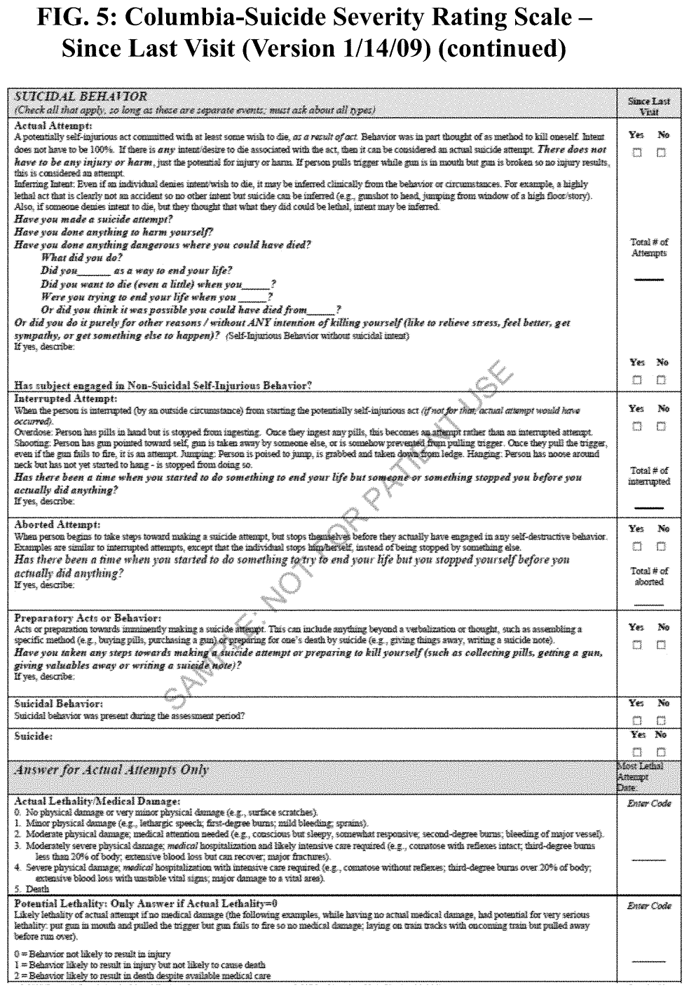

The Columbia-Suicide Severity Rating Scale (C-SSRS; FIG. 4 and FIG. 5) is a validated questionnaire used extensively across primary care, clinical practice, surveillance, research, and institutional settings to assess suicidal ideation and behavior (Posner. K. et al., Am. J. Psychiatry, 168:1266-77, 2011). The C-SSRS is administered by the Investigator or a properly trained designee. The C-SSRS is assessed as specified in the schedule of activities (Table 10 and Table 11). The C-SSRS is being implemented to ensure that patients who are experiencing suicidal ideation or behavior are properly recognized and adequately managed.

### Adverse Events and Serious Adverse Events

Adverse events are reported to the Investigator or qualified designee by the patient (or when appropriate, by a caregiver, surrogate, or the patient's legally authorized representative).

The Investigator or qualified designees are responsible for detecting, documenting, and recording events that meet the definition of an AE or SAE, and remain responsible for following up events that are serious, considered related to the study drug or study procedures; or that caused the patient to discontinue the study drug.

Time Period and Frequency for Collecting Adverse Event and Serious Adverse Event Information

All AEs are collected from the signing of the ICF until 8 weeks after the last dose of study drug is administered.

Medical occurrences that begin before the start of study drug, but after obtaining informed consent are recorded.

All SAEs are recorded and reported to the Sponsor or designee within 24 hours. The investigator submits any updated SAE data to the Sponsor within 24 hours of awareness.

Investigators are not obligated to actively seek AEs or SAEs after the conclusion of study participation. However, if the Investigator learns of any SAE, including a death, at any time after a patient has been discharged from the study, regardless of whether or not the event is related to the study drug, the Investigator promptly notifies the Sponsor.

Method of Detecting Adverse Events and Serious Adverse Events

Care is taken not to introduce bias when detecting AEs and/or SAEs. Open-ended and nonleading verbal questioning of the patient is the preferred method to inquire about AE occurrences.

Follow-Up of Adverse Events and Serious Adverse Events

After the initial AE/SAE report, the Investigator is required to proactively follow each patient at subsequent visits/contacts. All SAEs will be followed until resolution, stabilization, the event is otherwise explained, or the patient is lost to follow-up (as defined herein).

Regulatory Reporting Requirements for Serious Adverse Events
The Investigator notifies the Sponsor of an SAE within 24 hours of the first awareness of the event.
The Sponsor has a legal responsibility to notify both the local regulatory authority and other regulatory agencies about the safety of a study drug under clinical investigation. The Sponsor complies with country-specific regulatory requirements relating to safety reporting to the regulatory authority, IRB/IEC, and Investigators.
The Council for International Organizations of Medical Sciences (CIOMS) or MedWatch reports are prepared for suspected unexpected serious adverse reactions (SUSARs) according to local regulatory requirements and Sponsor policy and forwarded to Investigators as necessary. Alexion procedures for the reporting of SUSARs are in accordance with United States Title 21 Code of Federal Regulations (CFR) 312.32 and European Union Clinical Trial Directive 2001/20/EC and the associated detailed.
Guidance documents or national regulatory requirements in participating countries, as well as IRBs/IECs where applicable.
An Investigator who receives an Investigator safety report describing an SAE or other specific safety information (e.g., summary or listing of SAEs) from the Sponsor reviews and acknowledges the report and notifies the IRB/IEC, if appropriate, according to local requirements.

Pregnancy

For patients of childbearing potential, a serum pregnancy test (i.e., beta-human chorionic gonadotropin) is performed at Screening and at the EOS/ET. Urine pregnancy tests are performed at all other required time points, as indicated in the schedule of activities (Table 10 and Table 11). A negative pregnancy test is required prior to administering ravulizumab to patients of childbearing potential.

If a pregnancy is reported, the Investigator informs the Sponsor within 24 hours of learning of the pregnancy.

Abnormal pregnancy outcomes (e.g., spontaneous abortion, fetal death, stillbirth, congenital anomalies, and ectopic pregnancy) are considered SAEs and are reported.

Vaccine and Antibiotic Prophylaxis

As with any terminal complement antagonist, the use of ravulizumab increases the patient's susceptibility to meningococcal infection (N. meningitidis). To reduce the risk of meningococcal infection, all patients are vaccinated against meningococcal infections within the 3 years prior to, or at the time of, initiating study drug. Patients who initiate study drug treatment less than 2 weeks after receiving a meningococcal vaccine receive treatment with appropriate prophylactic antibiotics until 2 weeks after vaccination.

Vaccines against serotypes A, C, Y, W135, and B, where available, are recommended to prevent common pathogenic meningococcal serotypes. Patients are vaccinated or revaccinated according to current national vaccination guidelines or local practice for vaccination use with complement-inhibitors (e.g., eculizumab).

Vaccination may not be sufficient to prevent meningococcal infection. Consideration should be given per official guidance and local practice on the appropriate use of antibacterial agents. All patients are monitored for early signs of meningococcal infection, evaluated immediately if infection is suspected, and treated with appropriate antibiotics, if necessary.

To increase risk awareness and promote quick disclosure of any potential signs or symptoms of infection experienced by the patients during the course of the study, patients are provided a safety card to carry with them at all times. Additional discussion and explanation of the potential risks, signs, and symptoms occur at each visit as part of the review of the patient safety card as described in the schedule of activities (Table 10 and Table 11). Vaccination(s) for N meningitidis is recorded.

Study Drug Administration Reactions

Local and Systemic Reactions

Infusion-site reactions are those localized to the site of IV study drug administration and include those such as erythema, pruritus, and bruising. Infusion-associated reactions are those that are systemic in nature and that may be immune or nonimmune-mediated generally occurring within hours of study drug administration. Immune-mediated reactions include allergic reactions (e.g., anaphylaxis), while nonimmune-mediated reactions are nonspecific (e.g., headache, dizziness, nausea). Monitoring for these reactions are conducted as part of routine safety assessments for this study.

Infusion-Associated Reactions

Infusion-associated reactions are defined as systemic AEs (e.g., fever, chills, flushing, alterations in HR and BP, dyspnea, nausea, vomiting, diarrhea, and generalized skin rashes) occurring during or within 24 hours of the start of IV infusion that are assessed by the Investigator to be possibly, probably, or definitely related to the study drug.

### Adverse Events of Special Interest

Meningococcal infections are collected as adverse events of special interest (AESI) for this study.

### Pharmacokinetics

Blood samples are obtained to assess pre- and post-treatment serum ravulizumab concentrations at the time points and within the windows indicated in the schedule of activities (see, Table 10 and Table 11). Samples obtained outside of the allotted windows are considered protocol deviations. Unused samples are retained for a period of up to 5 years to perform additional assessments as necessary.

### Pharmacodynamics

Blood samples are obtained to assess pre- and post-treatment serum free C5 at the time points and within the windows indicated in the schedule of activities (Table 10 and Table 11). Samples obtained outside of the allotted windows are considered protocol deviations. Unused samples are retained for a period of up to 5 years to perform additional assessments as necessary.

### Biomarkers

Blood samples for the assessment of AChR auto-Abs are obtained at the time points indicated in the schedule of activities (Table 10 and Table 11).

### Healthcare Resource Utilization and Health Economics

Medical resource utilization and health economics data, associated with medical encounters, are collected by the Investigator or designee for all patients throughout the study. Data are recorded. Protocol-required procedures, tests, and encounters are excluded.

The data collected is used to conduct exploratory economic analyses and include:
Number and duration of medical care encounters, including surgeries, and other selected procedures (inpatient and outpatient);
Duration of hospitalization (total days or length of stay, including duration by wards (e.g., intensive care unit);
Number and type of diagnostic and therapeutic tests and procedures;
Outpatient medical encounters and treatments (including physician or emergency room visits, tests and procedures, and medications).

11. Statistical Considerations

Statistical methods described herein will be further elaborated in a separate SAP. The SAP is developed and finalized before database lock. The analyses are performed using the SAS® statistical software system Version 9.4 or later. Statistical analyses include tabulations of summary data, inferential analyses, by-patient listings and figures. Inference from efficacy analyses are based on 2-sided Type I error (α)=5%. Summary statistics for continuous variables minimally include n, mean, standard deviation, minimum, median, and maximum. For categorical variables, frequencies and percentages are presented.

The baseline value for analysis and reporting is based on the last nonmissing measurement on or prior to the first dose of study drug. The treatment groups for analysis and reporting are based on the conventions outlined in Table 18. A ‘Total’ group is formed to report demographics, baseline characteristics and other prestudy information such as prestudy SAEs, medical history, or prior medications. Details for imputation of efficacy data are described in the SAP. Missing safety data are not imputed.

Statistical Hypotheses

### Primary Hypothesis

The primary hypothesis for this study is that ravulizumab is superior to placebo in improvement of MG-ADL total score at Week 26.

The treatment effect based on the primary endpoint is estimated by the difference in means between the ravulizumab group and placebo group in the change from Baseline in MG-ADL total score at Week 26 irrespective of rescue therapyl. A lower value of the corresponding estimate indicates a beneficial treatment effect.

### Secondary Hypothesis

The following secondary hypothesis is included in study-wise multiplicity adjustment (provided the null hypothesis for primary endpoint is rejected) and as provided herein.

Ravulizumab is superior to placebo in improvement of QMG total score at Week 26.

### Hypotheses Related to Exploratory Efficacy Objectives

1. Ravulizumab is superior to placebo in reducing incidence of all-cause hospitalization or Clinical Deterioration over 26 weeks.
2. Ravulizumab is superior to placebo in improvement of the MG-QOL15r total score at Week 26.
3. Ravulizumab is superior to placebo in improvement of Neuro-QOL Fatigue total score at Week 26.
4. Ravulizumab is superior to placebo in improvement of the MGC total score at Week 26.
5. Ravulizumab is superior to placebo in QMG 5-point response (≥5 point improvement from baseline in QMG total score) at Week 26.
6. Ravulizumab is superior to placebo in MG-ADL 3-point response (≥3 point improvement from baseline in MG-ADL total score) at Week 26.
7. Ravulizumab is superior to placebo in MGFA-PIS at Week 26.
8. Ravulizumab is superior to placebo in improvement of EQ-5D-5L index score at Week 26.
9. Ravulizumab is superior to placebo in improvement of EQ-5D-5L VAS score at Week 26.

The treatment effect corresponding to the change from Baseline continuous endpoints is estimated similarly as the primary endpoint.

The treatment effect corresponding to the following dichotomous endpoints is estimated by the odds ratio (OR) of the proportions of the corresponding endpoint in the ravulizumab group compared with the placebo group:
a. Incidence of all-cause hospitalization or Clinical Deterioration over 26 weeks irrespective of rescue therapy.
b. QMG 5-point response at Week 26 irrespective of rescue therapy.
c. MG-ADL 3-point response at Week 26 irrespective of rescue therapy.

An estimate of OR<1 corresponding to the composite hospitalization endpoint indicates a beneficial treatment effect, likewise an estimate of OR>1 corresponding responder endpoints indicates a beneficial treatment effect.

The treatment effect corresponding to the MGFA-PIS endpoint is estimated by the proportional OR of the cumulative proportions over the ordinal categories (starting from the best outcome) of this endpoint in the ravulizumab group compared with the placebo group at Week 26, irrespective of rescue therapy. An estimate of OR>1 indicates a beneficial treatment effect.

### Sample Size Determination

Approximately 160 patients are randomly assigned to ravulizumab and placebo in a 1:1 ratio (ravulizumab:placebo) stratified by region (North America, Europe, Asia-Pacific, and Japan) to ensure at least 90% nominal power to reject the null hypotheses of no treatment difference for the primary and secondary endpoints based on 2-sided Type I error (a)=5%. Assumptions related to statistical power calculations are based on Study ECU-MG-301. Details are provided as defined herein.

TABLE 18
Study ALXN1210-MG-306 analysis sets

| Population | Description |
|---|---|
| Randominzed set | All randomized patients grouped by randomized treatment group (for reporting |
| | disposition, demographics, and baseline characteristics). |
| PK Analysis Set (PKAS) | All ravulizumab treated patients with at least 1 post-baseline PK concentration |
| | available. |
| Full analysis set (FAS) | All randomized patients who received at least 1 dose of study drug grouped by |
| | randomized treatment group (for reporting efficacy data). |
| Per protocol set (PPS) | Subset of FAS without any major protocol deviations¹ during |
| | Randomized-Controlled Period grouped by randomized treatment group (for |
| | reporting key efficacy data). |
| Safety set (SS) | All patients who received at least 1 dose of study drug grouped by treatment |
| | actually received (for reporting exposure and safety data). For a patient to be |
| | analyzed according to the treatment they actually received and not according to |
| | the randomization schedule, they would have to receive that treatment for the |
| | entire duration of Randomized-Controlled Period. |
| Open-label extension set | All patients who received at least 1 dose of ravulizumab starting from Week 26 |
| | onward (for reporting all data from the OLE Period). |

¹Determination of applicable major protocol deviations for this purpose will be made prior to database lock and study unblinding

### Statistical Analyses

Enrollment and Disposition

The number of patients screened, screen failures, and randomized patients are presented. Enrollment information is presented grouped by stratification factor and treatment group. Number of patients discontinued along with reasons from Randomized-Controlled Period, OLE Period, and the overall study is summarized.

Demographics. Baseline Characteristics, Inclusion and Exclusion Criteria, and Protocol Deviations

All demographic information and baseline characteristics are reported by treatment group and overall. No statistical test is performed for homogeneity among treatment groups.

The number and percentage of patients not meeting specific inclusion or exclusion criterion are summarized. Similar summary is provided for major protocol deviations based on prespecified categories.

Medical/Surgical History. Physical Examination, and Myasthenia Gravis History

The medical and surgical history is summarized by the Medical Dictionary for Regulatory (MedDRA) Activities, Version 20.1, or later by System Organ Class (SOC) and Preferred Term. MG and abnormal physical examination are also summarized.

Prior and Concomitant Medications

For analysis and reporting purpose, any medication started prior to first dose of study drug is considered as prior medication; and medications that started on or after the first dose of study drug are considered as concomitant medications. All prior and concomitant medications including MG-specific medications and rescue therapy during the study, if any, are summarized.

Efficacy Analyses

Primary Efficacy Analysis

The Mixed-effects Model with Repeated Measures (MMRM) is used for the primary efficacy endpoint (change from Baseline in MG-ADL total score at Week 26) using all available longitudinal data (either complete or partial) regardless of whether patients received a rescue therapy. Rescue therapy includes high-dose corticosteroids, PP/PE or IVIg. It is allowed when a patient's health is in jeopardy, if rescue therapy is not administered (e.g., emergent situations), or if a patient experiences clinical deterioration. Missing data is not imputed for the primary analysis. The model includes the MG-ADL change from Baseline score at each prespecified time point as the response variable, fixed categorical effects of treatment, study visit and treatment-by-study visit interaction, region; as well as fixed covariate of baseline MG-ADL total score. The treatment effect is evaluated via contrast for the treatment-by-visit term at Week 26. An unstructured covariance matrix is used to model the correlations among repeated measurements within each patient. Other covariance structures are implemented if a convergence issue occurs (details to be provided in SAP). The Kenward-Rogers method is used to estimate the denominator degrees of freedom.

Sensitivity Analyses for Primary Endpoint

Two sensitivity analyses are performed for the primary efficacy endpoint to explore the robustness of the MMRM results for the primary efficacy analysis:

1. Placebo-based sensitivity analysis:
The placebo-based sensitivity analysis considers the Missing Not At Random (MNAR) mechanism for the missing data, where it is assumed that patients who discontinue early from ravulizumab follow the trajectory of outcomes similar to the one in the placebo group after discontinuation of ravulizumab, taking into account observed values prior to discontinuation.

2. Tipping point sensitivity analysis:
This approach assumes that patients who discontinue from ravulizumab treatment experience worsening defined by a prespecified adjustment in the primary efficacy endpoint.

Analyses of Secondary and Exploratory Endpoints

All continuous secondary and exploratory endpoints related to change from Baseline are analyzed similarly as the primary endpoint.

The composite endpoint of Clinical Deterioration or all-cause hospitalization is analyzed using a logistic regression model with treatment group, region. The individual components (clinical deterioration and all-cause hospitalization separately) are also analyzed in similar fashion.

The QMG 5-point and MG-ADL 3-point responder endpoints are analyzed using a mixed effect repeated measures model. The model includes response variable at each pre-specified time point as the dependent variable, fixed categorical effects of treatment, study visit and treatment-by-study visit interaction, and region; as well as fixed covariate of baseline QMG or MG-ADL total score (depending on the response variable). The treatment effect is evaluated via contrast for the treatment-by-visit term at Week 26. An unstructured covariance matrix is used to model the correlations among repeated measurements within each patient. Other covariance structures are implemented if a convergence issue occurs (details to be provided in SAP).

The MGFA-PIS endpoint at Week 26 is considered as an ordinal scale. A logistic regression of the cumulative odds (cumulated over the categories starting from best outcome) is performed using treatment as fixed categorical effect and adjusting for region.

Long-term efficacy data is summarized descriptively based on OLE set.

Multiplicity Adjustment for Primary and Secondary Endpoints

The study is designed to strongly control the overall 2-sided Type I error of α=0.05. The primary null hypothesis is tested first at α=0.05. If statistically significant, the secondary efficacy hypothesis is tested at α=0.05.

Per Protocol Analyses for Primary and Secondary Endpoints

Supplemental per protocol analyses for primary and secondary endpoints are performed based on per protocol set (PPS) in the same manner as done for FAS.

Safety Analyses

The safety and tolerability of ravulizumab is assessed based on adverse events, clinical laboratory findings, vital sign findings, and ECG abnormalities. Safety analyses are performed on the Safety Population and OLE set based on the study period under consideration.

Analysis of Adverse Events

Analysis and reporting for AEs are based on treatment-emergent adverse events (TEAEs), including treatment-emergent serious adverse events (TESAEs) defined as an AE with onset on or after first dose of ravulizumab in the Randomized-Controlled Period. Treatment-emergent AEs and TESAEs are summarized by MedDRA SOC and Preferred Term, by severity, and by relationship to the study drug. Patient-years adjusted event rates are generated to characterize long-term safety profile.

Analysis of Clinical Laboratory Parameters, Vital Sign Measurements and Electrocardiogram Parameters

Laboratory measurements as well as their changes from Baseline at each visit and shift from baseline, if applicable, are summarized descriptively. Significant ECG, vital sign, and pulse oximetry findings are also summarized using descriptive analyses.

Other Safety Analyses

The number and percentage of patients in each of the C-SSRS categories and shift analyses are produced. Results from pregnancy tests are summarized.

Analysis of Pharmacokinetics and Pharmacodynamics

Pharmacokinetic parameters such as peak and trough serum ravulizumab concentrations are reported and summarized. Population PK analysis of ravulizumab are performed to characterize the PK of ravulizumab in patients with gMG using the sparse PK data. Key ravulizumab PK parameters such as clearance, volume of distribution, and terminal half-life are estimated using the population-PK analysis. The potential impact of intrinsic and extrinsic factors on ravulizumab PK are also assessed. Pharmacodynamic data (pre- and post-treatment free C5) are reported and summarized. Correlations between PK and PD are explored. Additional analyses are considered, if appropriate.

Analysis of Immunogenicity

The presence of ADAs in serum ravulizumab are assessed over the duration of the study. Immunogenicity results are analyzed by summarizing the number and percentage of patients who develop detectable ADA. The association of ADA with ravulizumab concentration, PD parameters, efficacy, and TEAEs are evaluated.

Analysis of Exploratory Biomarkers

Acetylcholine receptor antibody titer levels as well as their changes from Baseline at each visit are summarized descriptively.

Interim Analyses

No interim analysis is planned for Study ALXN1210-MG-306 during the Randomized-Controlled Period. The primary analysis is conducted when the last patient completes the Randomized-Controlled Period, the database is locked, and the study randomization schedule is unblinded. Periodic analysis and reporting is performed during the OLE Period based on regulatory requirement. Final analysis and reporting is conducted at the conclusion of the study.

Additional Details on Sample Size Determination

The power calculations are based on the longitudinal change from baseline in MG-ADL total score observed in Study ECU-MG-301. A simulation-based approach is adopted to calculate the power based on the model-based treatment effect in MG-ADL. A total of 160 patients are required to ensure at least 90% power to reject the null hypothesis of no treatment effect based on the change from Baseline in MG-ADL total score at Week 26. Further details are provided in the SAP.

Additional Details on Sensitivity Analysis for Primary Endpoint

To assess the credibility of the primary analysis, the following sensitivity analyses are planned, based on assumptions that are unfavorable enough to the ravulizumab group to constitute a convincing stress test of the primary analysis.

Placebo-Based Sensitivity Analysis

The placebo-based sensitivity analysis considers the MNAR mechanism for the missing data, where it is assumed that patients who discontinue early from the ravulizumab group follow the trajectory of outcomes similar to the one in the placebo group after discontinuation of ravulizumab, taking into account observed values prior to discontinuation (Little, R. & Yau, L., Biometrics, 52:1324-33, 1996; Ratitch, B. et al., Pharm. Stat., 12:337-47, 2013). Patients discontinuing early from placebo are assumed to have unobserved outcomes similar to placebo patients who remain on their randomized treatment. The assumption that the efficacy profiles of dropouts after discontinuation of ravulizumab are similar to those of patients in the placebo group provides an estimate of efficacy attributable to patients in the ravulizumab group if received through the time point of interest, while limiting efficacy after early discontinuation to that of the placebo group.

Tipping Point Sensitivity Analysis

An additional sensitivity analysis is performed based on the delta-adjusted stress testing method (tipping point analysis). This approach assumes that patients who discontinue from the active treatment experience worsening defined by a prespecified adjustment (delta) in the primary efficacy endpoint compared with the observed efficacy score of patients that continue the study to next visit (O'Kelley M R B, Statistics in Practice. 1 ed. Chichester, West Sussex, UK: John Wiley & Sons, Ltd; 2014. p. 257-368). Since a negative change in QMG total score indicates improvement, the prespecified value of delta is a non-negative fixed quantity. For each value of delta, the treatment effect is determined and the value of delta for which the nominal 2-sided p-value crosses 0.05, is considered as the ‘tipping point’ in the sense that the positive conclusion drawn from the primary analysis is reversed when patients who drop out are assumed to experience this fixed worsening after the discontinuation visit. After such a tipping point is determined, clinical judgment is applied as to the plausibility of the assumptions underlying this tipping point. This methodology is expected to inform of what it would take to overturn study conclusions based on varying assumptions about missing data. A value of delta as zero is considered equivalent to the primary analysis.

SEQUENCE SUMMARY
SEQ ID NO: 1
GYIFSNYWIQ
SEQ ID NO: 2
EILPGSGSTEYTENFKD
SEQ ID NO: 3
YFFGSSPNWYFDV
SEQ ID NO: 4
GASENIYGALN
SEQ ID NO: 5
GATNLAD
SEQ ID NO: 6
QNVLNTPLT
SEQ ID NO: 7
QVQLVQSGAEVKKPGASVKVSCKASGYIFSNYWIQWVRQAPGQGLEWMGEILPGSGSTEYTEN
FKDRVTMTRDTSTSTVYMELSSLRSEDTAVYYCARYFFGSSPNWYFDVWGQGTLVTVSS
SEQ ID NO: 8
DIQMTQSPSSLSASVGDRVTITCGASENIYGALNWYQQKPGKAPKLLIYGATNLADGVPSRFS
GSGSGTDFTLTISSLQPEDFATYYCQNVLNTPLTFGQGTKVEIK
SEQ ID NO: 9
ASTKGPSVFPLAPCSRSTSESTAALGCLVKDYFPEPVTVSWNSGALTSGVHYFPAVLQSSGLY
SLSSVVTVPSSNFGTQTYTCNVDHKPSNTKVDKTVERKCCVECPPCPAPPVAGPSVFLFPPKP
KDTLIMSRTPEVTCVVVDVSQEDPEVQFNWYVDGVEVHNAKTKPREEQFNSTYRVVSVLTVLH
QDWLNGKEYKCKVSNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVFSCSVMHEALHNHY
TQKSLSLSLGK
SEQ ID NO: 10
QVQLVQSGAEVKKPGASVKVSCKASGYIFSNYWIQWVRQAPGQGLEWMGEILPGSGSTEYTEN
FKDRVTMTRDTSTSTVYMELSSLRSEDTAVYYCARYFFGSSPNWYFDVWGQGTLVTVSSASTK
GPSVFPLAPCSRSTSESTAALGCLVKDYFPEPVTVSWNSGALTSGVHTFPAVLQSSGLYSLSS
VVTVPSSNFGTQTYTCNVDHKPSNTKVDKTVERKCCVECPPCPAPPVAGPSVFLFPPKPKDTL
MISRTPEVTCVVVDVSQEDPEVQFNWYVDGVEVHNAKTKPREEQFNSTYRVVSVLTVLHQDWL
NGKEYKCKVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEALHNHYTQKS
LSLSLGK
SEQ ID NO: 11
DIQMTQSPSSLSASVGDRVTITCGASENIYGALNWYQQKPGKAPKLLIYGATNIADGVPSRFS
GSGSGTDFTLTISSLQPEDFATYYCQNVLNTPLTFGQGTKVEIKRTVAAPSVFIFPPSDEQLK
SGTASVVCLLNNFYPREAKVQWKVDNALQSGNSQESVTEQDSKDSTYSLSSTLTLSKADYEKH
KVYACEVTHQGLSSPVTKSFNRGEC
SEQ ID NO: 12
QVQLVQSGAEVKKPGASVKVSCKASGHIFSNYWIQWVRQAPGQGLEWMGEILPGSGHTEYTEN
FKDRVTMTRDTSTSTVYMELSSLRSEDTAVYYCARYFFGSSPNWYFDVWGQGTLVTVSS
SEQ ID NO: 13
ASTKGPSVFPLAPCSRSTSESTAALGCLVKDYFPEPVTVSWNSGALTSGVHTFPAVLQSSGLY
SLSSVVTVPSSNFGTQTYTCNVDHKPSNTKVDKTVERKCCVECPPCPAPPVAGPSVFLFPPKP
KDTLMISRTPEVTCVVVDVSQEDPEVQFNWYVDGVEVHNAKTKPREEQFNSTYRVVSVLTVLH
QDWLNGKEYKCKVSNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVFSCSVLHEALHSHY
TQKSLSLSLGK
SEQ ID NO: 14
QVQLVQSGAEVKKPGASVKVSCKASGHIFSNYWIQWVRQAPGQGLEWMGEILPGSGHTEYTEN
FKDRVTMTRDTSTSTVYMELSSLRSEDTAVYYCARYFFGSSPNWYFDVWGQGTLVTVSSASTK
GPSVFPLAPCSRSTSESTAALGCLVKDYFPEPVTVSWNSGALTSGVHTFPAVLQSSGLYSLSS
VVTVPSSNFGTQTYTCNVDHKPSNTKVDKTVERKCCVECPPCPAPPVAGPSVFLFPPKPKDTL
MISRTPEVTCVVVDVSQEDPEVQFNWYVDGVEVHNAKTKPREEQFNSTYRVVSVLTVLHQDWL
NGKEYKCKVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSV [symbol] HEALH [symbol] HYTQKS
LSLSLGK
SEQ ID NO: 15
ASTKGPSVFPLAPCSRSTSESTAALGCLVKDYFPEPVIVSWNSGALTSGVHTFPAVLQSSGLY
SLSSVVIVTSSNFGTQTYTCNVDHKPSNIKVDKTVERKCCVECPPCPAPPVAGPSVFLYPPKP
KDTLYITREPEVTCVVVDVSHEDPEVQYNWYVDGMEVENAKTKPREEQFNSTFRVVEVLTVVH
QDWLNGKEYKCKVSNKGLPAPIEKTISKTKGQPREPQVYTLPPSREEMTKNQVSLTCLVKGFY
PSDIAVEWESNGQPENNYKTTPPMLDSDGSFFLYSKLTVDKSRWQQGNVFSCSVMHEALHNHY
TQKSLSLSPGK
SEQ ID NO: 16
QVQLVQSGAEVKKPGASVKVSCKASGYIFSNYWIQWVRQAPGQGLEWMGEILPGSGSTEYTEN
FKDRVTMTRDTSTSTVYMELSSLRSEDTAVYYCARYFFGSSPNWYFDVWGQGTLVTVSSASTK
GPSVFPLAPCSRSTSESTAALGCLVKDYFPEPVTVSWNSGALTSGVHTFPAVLQSSGLYSLSS
VVTVTSSNFGTQTYTCNVDHKPSNTKVDKTVERKCCVECPPCPAPPVAGPSVFLFPPKPKDTL
YITREPEVTCVVVDVSHEDPEVQFNWYVDGMEVHNAKTKPREEQFNSTFRVVSVLTVVHQDWL
NGKEYKCKVSNKGLPAPIEKTISKTKGQPREPQVYTLPPSREEMTKNQVSLTCLVKGFYPSDI
AVEWESNGQPENNYKTTPPMLDSDGSFFLYSKLTVDKSRWQQGNVFSCSVMHEALHNHYTQKS
LSLSPGK
SEQ ID NO: 17
GASENIYHALN
SEQ ID NO: 18
EILPGSGHTEYTENFKD
SEQ ID NO: 19
GHIFSNYWIQ
SEQ ID NO: 20
QVQLVQSGAEVKKPGASVKVSCKASGHIFSNYWIQWVRQAPGQGLEWMGEILPGSGHTEYTEN
FKDRVTMTRDTSTSTVYMELSSLRSEDTAVYYCARYFFGSSPNWYFDVWGQGTLVTVSSASTK
GPSVFPLAPCSRSTSESTAALGCLVKDYFPEPVTVSWNSGALTSGVHTFPAVLQSSGLYSLSS
VVTVPSSNFGTQTYTCNVDHKPSNTKVDKTVERKCCVECPPCPAPPVAGPSVFLFPPKPKDTL
MISRTPEVTCVVVDVSQEDPEVQFNWYVDGVEVHNAKTKPREEQFNSTYRVVSVLTVLHQDWL
NGKEYKCKVSNKGLPSSIEKTISKAKGQPREFQVYTLPPSQEEMTKNQVSLTCLVKGFYPSDI
AVEWESNGQPENNYKTTPPVLDSDGSFFLYSRLTVDKSRWQEGNVFSCSV [symbol] HEALH [symbol] HYTQKS
LSLSLGK
SEQ ID NO: 21
SYAIS
SEQ ID NO: 22
GIGPFFGTANYAQKFQG
SEQ ID NO: 23
DTPYFDY
SEQ ID NO: 24
SGDSIPNYYVY
SEQ ID NO: 25
DDSNRPS
SEQ ID NO: 26
QSFDSSLNAEV
SEQ ID NO: 27
QVQLVQSGAEVKKPGSSVKVSCKASGGTFSSYAISVWRQAPGQGLEWMGGIGPFFGTANYAQK
FQGRVTITADESTSTAYMELSSLRSEDTAVYYCARDTPYFDYWGQGTLVTVSS
SEQ ID NO: 28
DIELTQPPSVSVAPGQTARISCSGDSIPNYYVYWYQQKPGQAPVLVIYDDSNRPSGIPERFSG
SNSGNTATLTISGTQAEDEADYYCQSFDSSLNAEVFGGGTKLTVL
SEQ ID NO: 29
NYIS
SEQ ID NO: 30
IIDPDDSYTEYSPSFQG
SEQ ID NO: 31
YEYGGFDI
SEQ ID NO: 32
SGDNIGNSYVH
SEQ ID NO: 33
KDNDRPS
SEQ ID NO: 34
GTYDIESYV
SEQ ID NO: 35
EVQLVQSGAEVKKPGESLKISCKGSGYSFTNYISWVRQMPGKGLEWMGIIDPDDSYTEYSPSF
QGQVTISADKSISTAYLQWSSLKASDTAMYYCARYEYGGFDIWGQGTLVTVSS
SEQ ID NO: 36
SYELTQPPSVSVAPGQTARISCSGDNIGNSYVHWYQQKPGQAPVLVIYKDNDRPSGIPERFSG
SNSGNTATLTISGTQAEDEADYYCGTYDIESYVFGGGTKLTVL
SEQ ID NO: 37
SSYYVA
SEQ ID NO: 38
AIYTGSGATYKASWAKG
SEQ ID NO: 39
DGGYDYPTHAMHY
SEQ ID NO: 40
QASQNIGSSLA
SEQ ID NO: 41
GASKTHS
SEQ ID NO: 42
QSTKVGSSYGNH
SEQ ID NO: 43
QVQLVESGGGLVQPGGSLRLSCAASGFTSHSSYYVAWVRQAPGKGLEWGAIYTGSGATYKAS
WAKGRFTISKDTSKNQVVLTMTKMDPVDTATYYCASDGGYDYPTHAMHYWGQGTLVTVSS
SEQ ID NO: 44
DVVMTQSPSSLSASVGDRVTITCQASQNIGSSLAWYQQKPGQAPRLLIYGASKTHSGVPSRFS
GSGSGTDFTLTISSLQPEDVATYYCQSTKVGSSYGNHFGGGTKVEIK
SEQ ID NO: 45
QVQLVESGGGLVQPGRSLRLSCAASGFTVHSSYYMAWVRQAPGKGLEWVGAIFTGSGAEYKAE
WAKGRVTISKDTSKNQVVLTMTNMDPVDTATYYCASDAGYDYPTHAMHYWGQGTLVTVSSAST
KGPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAPELRRGPKVFLFPPK
PKDTLMISRTPEVTCVVVDVSHEDPEVKFNWYVDGVEVHNAKTKPREEQYNSTYRVVSVLTVL
HQDWLNGKEYKCKVSNKGLPSSIEKTISKAKGQPREPQVYTLPPSREEMTKNQVSLTCLVKGF
YPSDIAVEWESNGQPENNYKTTPPVLDSDGSFFLYSKLTVDKSRWQQGNVFSCSVLHEALHAH
YTRKELSLSP
SEQ ID NO: 46
DIQMTQSPSSLSASVGDRVTITCRASQGISSSLAWYQQKPGKAPKLLIYGASETESGVPSRFS
GSGSGTDFTLTISSLQPEDFATYYCQNTKVGSSYGNTFGGGTKVEIKRTVAAPSVFIFPPSDE
QLKSGTASVVCLLNNFYPREAKVQWKVDNALQSGNSQESVTEQDSKDSTYSLSSTLTLSKADY
EKHKVYACEVTHQGLSSPVTKSFNRGEC
SEQ ID NO: 47
QVQLQESGPGLVKPSETLSLTCTVSGDSVSSSYWTWIRQPPGKGLEWIGYIYYSGSSNYNPSL
KSRATISVDTSKNQFSLKLSSVTAADTAVYYCAREGNVDTTMIFDYWGQGTLVTVSS
SEQ ID NO: 48
AIQMTQSPSSLSASVGDRVTITCRASQGIRNDLGWYQQKPGKAPKLLIYAASSLQSGVPSRFA
GRGSGTDFTLTISSLQPEDFATYYCLQDFNYPWTFGQGTKVEIK
SEQ ID NO: 49
QVQLQESGPGLVKPSETLSLTCTVSGDSVSSSYWTWIRQPPGKGLEWIGYIYYSGSSNYNPSL
KSRATISVDTSKNQFSLKLSSVTAADTAVYYCAREGNVDTTMIFDYWGQGTLVTVSSASTKGP
SVFPLAPCSRSTSESTAALGCLVKDYFPEPVTVSWNSGALTSGVHTFPAVLQSSGLYSLSSVV
TVPSSSLGTKTYTCNVDHKPSNTKVDKRVESKYGPPCPPCPAPEFLGGPSVFLFPPKPKDTLM
ISRTPEVTCVVVDVSQEDPEVQFNWYVDGVEVHNAKTKPREEQFNSTYRVVSVLTVLHQDWLN
GKEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYTQKSL
SLSLGK
SEQ ID NO: 50
AIQMTQSPSSLSASVGDRVTITCRASQGIRNDLGWYQQKPGKAPKLLIYAASSLQSGVPSRFA
GRGSGTDFTLTISSLQPEDFATYYCLQDFNYPWTFGQGTKVEIKRTVAAPSVFIFPPSDEQLK
SGTASVVCLLNNFYPREAKVQWKVDNALQSGNSQESVTEQDSKDSTYSLSSTLTLSKADYEKH
KVYACEVTHQGLSSPVTKSFNRGEC

1-31. (canceled)

32. A kit for treating myasthenia gravis (MG) in a human patient, the kit comprising
a dose of an antibody or an antigen binding fragment thereof comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8.

33. The kit of claim 32, wherein the antibody or the antigen binding fragment thereof comprises a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434, each in EU numbering.

34-36. (canceled)

37. The kit of claim 32, wherein
the antibody is ravulizumab.

38-42. (canceled)

43. A method of treating a human patient with myasthenia gravis (MG), the method comprising administering to the patient an effective amount of an antibody or an antigen binding fragment thereof comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively.

44. The method of claim 43, wherein the antibody or the antigen binding fragment thereof comprises a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434, each in EU numbering.

45. The method of claim 43, wherein the antibody or the antigen binding fragment thereof is administered in a treatment cycle, wherein the antibody or the antigen binding fragment thereof is administered:
(a) once on Day 1 of the administration cycle at a loading dose of:
i. 2400 mg to a patient weighing ≥40 to <60 kg,
ii. 2700 mg to a patient weighing ≥60 to <100 kg, or
iii. 3000 mg to a patient weighing ≥100 kg; and
(b) on Day 15 of the administration cycle and every eight weeks thereafter at a maintenance dose of:
i. 3000 mg to a patient weighing ≥40 to <60 kg,
ii. 3300 mg to a patient weighing ≥60 to <100 kg, or
iii. 3600 mg to a patient weighing ≥100 kg.

46. The of claim 43, wherein the antibody or the antigen binding fragment thereof comprises the heavy chain variable region of SEQ ID NO:12 and the light chain variable region of SEQ ID NO:8.

47. The method of claim 46, wherein the antibody or the antigen binding fragment thereof further comprises the heavy chain constant region of SEQ ID NO:13.

48-53. (canceled)

54. The method of claim 45, wherein the treatment maintains a serum trough concentration of the antibody or the antigen binding fragment thereof of 100 μg/mL or greater during the administration cycle.

55-56. (canceled)

57. The method of claim 45, wherein the antibody or the antigen binding fragment thereof is administered at a dose of 3000 mg, 3300 mg or 3600 mg every eight weeks after the administration cycle for up to two years.

58. (canceled)

59. The method of claim 43, wherein the patient has not previously been treated with a complement inhibitor.

60. The method of claim 45, wherein the administration cycle is a total of 26 weeks of treatment.

61. The method of claim 43, wherein the treatment results in terminal complement inhibition.

62. The method of claim 43, wherein the treatment results in the patient experiencing a clinically meaningful improvement (reduction) in Myasthenia Gravis Activities of Daily Living (MG-ADL) score, a reduction in Myasthenia Gravis Composite (MGC) score, a reduction Myasthenia Gravis Quality of Life (MG-QOL15r) score, a reduction in Neuro-QOL Fatigue score, a reduction in Euro Quality of Life (EQ-5D-5L) health status score, or a reduction in Myasthenia Gravis Foundation of America (MGFA) Post-Intervention Status (PIS) after 26 weeks of treatment.

63. The method of claim 62, wherein the clinically meaningful improvement the patient experiences is at least a 3 point reduction in the patient's MG-ADL score after 26 weeks of treatment.

64. (canceled)

65. The method of claim 62, wherein the clinically meaningful improvement the patient experiences is at least a 5 point reduction in the patient's QMG after 26 weeks of treatment.

66-71. (canceled)

72. The method of claim 43, wherein the MG patient is anti-AChR antibody positive.

73. (canceled)


SEQUENCE LISTING (50 sequences)

SEQ ID NO: 1 - protein, 10 aa, Artificial Sequence
  Heavy Chain CDR Sequence
GYIFSNYWIQ

SEQ ID NO: 2 - protein, 17 aa, Artificial Sequence
  Heavy Chain CDR Sequence
EILPGSGSTEYTENFKD

SEQ ID NO: 3 - protein, 13 aa, Artificial Sequence
  Heavy Chain CDR Sequence
YFFGSSPNWYFDV

SEQ ID NO: 4 - protein, 11 aa, Artificial Sequence
  Light Chain CDR Sequence
GASENIYGALN

SEQ ID NO: 5 - protein, 7 aa, Artificial Sequence
  Light Chain CDR Sequence
GATNLAD

SEQ ID NO: 6 - protein, 9 aa, Artificial Sequence
  Light Chain CDR Sequence
QNVLNTPLT

SEQ ID NO: 7 - protein, 122 aa, Artificial Sequence [sequence omitted]

SEQ ID NO: 8 - protein, 107 aa, Artificial Sequence [sequence omitted]

SEQ ID NO: 9 - protein, 326 aa, Artificial Sequence
  Heavy Chain Constant Region Sequence
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 448 aa, Artificial Sequence
  Heavy Chain Sequence
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, Artificial Sequence [sequence omitted]

SEQ ID NO: 12 - protein, 122 aa, Artificial Sequence [sequence omitted]

SEQ ID NO: 13 - protein, 326 aa, Artificial Sequence
  Heavy Chain Constant Region Sequence
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 14 - protein, 448 aa, Artificial Sequence
  Heavy Chain Sequence
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 15 - protein, 326 aa, Artificial Sequence
  Heavy Chain Constant Region Sequence
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - protein, 448 aa, Artificial Sequence
  Heavy Chain Sequence
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 11 aa, Artificial Sequence
  Heavy Chain CDR Sequence
GASENIYHALN

SEQ ID NO: 18 - protein, 17 aa, Artificial Sequence
  Heavy Chain CDR Sequence
EILPGSGHTEYTENFKD

SEQ ID NO: 19 - protein, 10 aa, Artificial Sequence
  Heavy Chain CDR Sequence
GHIFSNYWIQ

SEQ ID NO: 20 - protein, 448 aa, Artificial Sequence
  Heavy Chain Sequence
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 5 aa, Artificial Sequence
  Heavy Chain CDR Sequence
SYAIS

SEQ ID NO: 22 - protein, 17 aa, Artificial Sequence
  Heavy Chain CDR Sequence
GIGPFFGTANYAQKFQG

SEQ ID NO: 23 - protein, 7 aa, Artificial Sequence
  Heavy Chain CDR Sequence
DTPYFDY

SEQ ID NO: 24 - protein, 11 aa, Artificial Sequence
  Light Chain CDR Sequence
SGDSIPNYYVY

SEQ ID NO: 25 - protein, 7 aa, Artificial Sequence
  Light Chain CDR Sequence
DDSNRPS

SEQ ID NO: 26 - protein, 11 aa, Artificial Sequence
  Light Chain CDR Sequence
QSFDSSLNAEV

SEQ ID NO: 27 - protein, 116 aa, Artificial Sequence
  Heavy Chain Variable Region Sequence
QVQLVQSGAEVKKPGSSVKVSCKASGGTFSSYAISVWRQAPGQGLEWMGGIGPFFGTANYAQKFQGRVTITADESTSTAYMELSSLRSEDTAVYYCARDTPYFDYWGQGTLVTVSS

SEQ ID NO: 28 - protein, 108 aa, Artificial Sequence
  Light Chain Variable Region Sequence
DIELTQPPSVSVAPGQTARISCSGDSIPNYYVYWYQQKPGQAPVLVIYDDSNRPSGIPERFSGSNSGNTATLTISGTQAEDEADYYCQSFDSSLNAEVFGGGTKLTVL

SEQ ID NO: 29 - protein, 4 aa, Artificial Sequence
  Heavy Chain CDR Sequence
NYIS

SEQ ID NO: 30 - protein, 17 aa, Artificial Sequence
  Heavy Chain CDR Sequence
IIDPDDSYTEYSPSFQG

SEQ ID NO: 31 - protein, 8 aa, Artificial Sequence
  Heavy Chain CDR Sequence
YEYGGFDI

SEQ ID NO: 32 - protein, 11 aa, Artificial Sequence
  Light Chain CDR Sequence
SGDNIGNSYVH

SEQ ID NO: 33 - protein, 7 aa, Artificial Sequence
  Light Chain CDR Sequence
KDNDRPS

SEQ ID NO: 34 - protein, 9 aa, Artificial Sequence
  Light Chain CDR Sequence
GTYDIESYV

SEQ ID NO: 35 - protein, 116 aa, Artificial Sequence
  Heavy Chain Variable Region Sequence
EVQLVQSGAEVKKPGESLKISCKGSGYSFTNYISWVRQMPGKGLEWMGIIDPDDSYTEYSPSFQGQVTISADKSISTAYLQWSSLKASDTAMYYCARYEYGGFDIWGQGTLVTVSS

SEQ ID NO: 36 - protein, 106 aa, Artificial Sequence
  Light Chain Variable Region Sequence
SYELTQPPSVSVAPGQTARISCSGDNIGNSYVHWYQQKPGQAPVLVIYKDNDRPSGIPERFSGSNSGNTATLTISGTQAEDEADYYCGTYDIESYVFGGGTKLTVL

SEQ ID NO: 37 - protein, 6 aa, Artificial Sequence
  Heavy Chain CDR Sequence
SSYYVA

SEQ ID NO: 38 - protein, 17 aa, Artificial Sequence
  Heavy Chain CDR Sequence
AIYTGSGATYKASWAKG

SEQ ID NO: 39 - protein, 13 aa, Artificial Sequence
  Heavy Chain CDR Sequence
DGGYDYPTHAMHY

SEQ ID NO: 40 - protein, 11 aa, Artificial Sequence
  Light Chain CDR Sequence
QASQNIGSSLA

SEQ ID NO: 41 - protein, 7 aa, Artificial Sequence
  Light Chain CDR Sequence
GASKTHS

SEQ ID NO: 42 - protein, 12 aa, Artificial Sequence
  Light Chain CDR Sequence
QSTKVGSSYGNH

SEQ ID NO: 43 - protein, 123 aa, Artificial Sequence
  Heavy Chain Variable Region Sequence
QVQLVESGGGLVQPGGSLRLSCAASGFTSHSSYYVAWVRQAPGKGLEWVGAIYTGSGATYKASWAKGRFTISKDTSKNQVVLTMTNMDPVDTATYYCASDGGYDYPTHAMHYWGQGTLVTVSS

SEQ ID NO: 44 - protein, 110 aa, Artificial Sequence
  Light Chain Variable Region Sequence
DVVMTQSPSSLSASVGDRVTITCQASQNIGSSLAWYQQKPGQAPRLLIYGASKTHSGVPSRFSGSGSGTDFTLTISSLQPEDVATYYCQSTKVGSSYGNHFGGGTKVEIK

SEQ ID NO: 45 - protein, 451 aa, Artificial Sequence
  Heavy Chain Sequence
QVQLVESGGGLVQPGRSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 46 - protein, 217 aa, Artificial Sequence
  Light Chain Sequence
DIQMTQSPSSLSASVGDRVTITCRASQGISSSLAWYQQKPGKAPKLLIYGASETESGVPSRFSGSGSGTDFTLTISSLQPEDFATYYCQNTKVGSSYGNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 120 aa, Artificial Sequence
  Heavy Chain Variable Region Sequence
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSS

SEQ ID NO: 48 - protein, 107 aa, Artificial Sequence
  Light Chain Variable Region Sequence
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIK

SEQ ID NO: 49 - protein, 447 aa, Artificial Sequence
  Heavy Chain Sequence
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 214 aa, Artificial Sequence
  Light Chain Sequence
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC